SEQUENCE LISTING (63 sequences)

SEQ ID NO: 1 - protein, 330 aa, Nicotiana sp.
MESKSSGGGEGKVVCVTGASGFIASWLVKMLLQRGYTVNATVRNLKDASKVDHLLGLDGAKERLHLFKAELLGEHSFDPAVDGCEGVFHTASPVSLTAKSKEELVDPAVSGTLNVLRSCTKSTSVRRVVITSSTASVICNKNMSTPGAVADETWYSDAELCEERKEWYQLSKTLAEEAAWKFAKENGLDLVTLHPGLVIGPLLQPTLNFSCEAIVNFIKEGKEAWSGGIYRFVDVRDVANAHILAFEVPSANGRYCLVGVNGYSSLVLKIVQKLYPSITLPENFEDGLPLIPTFQVSSERAKSLGVNFTSLELSVKDTVESLIEKNFLKI

SEQ ID NO: 2 - protein, 416 aa, Nicotiana sp.
MMCNIISLVSIINFFLLFYRVVVLPQHAAASHSTVEFLPGFEGPLPFHLETGYIGVGEYEEVQLFYYFLKSESEPTKDPILIWLSGGPGCSSFTALVYQIGPLYFEPNEYNGSLPKLTLNPNSWTKVANIIFLDQPVNSGFSYATTSTTFKSTDLQACHHIYQFLRKWLIKHQEFIRNPMYIGGDSYSGITVPVITQLISNGIEAGHKPSINLKGYILGNPSTFPLQYNYWVPYAHGMGLISDELYQATTLILYFKIYHIINAINLAYMVEVHRLSTYWANDPRVQEALNVRKGAITRWTRCRESIVNKTYTITFQDSIPYHVELSKKLYRSLIYSGDHDMGIPFQSTQFWIKSLNYSIVDEWRPWSFDGQVAGYTRSYSNQMTFATVKGAGHVAPEYKPKECFTMFQRWLSHEPL

SEQ ID NO: 3 - protein, 569 aa, Nicotiana sp.
MLSVASVEAQKAELSSSVIPPSDQSTEEIHVFRSRLPDIQISNNVPLHVYLFERLSEFQDRTCLIAGSSGQSYTFAETHLICQKIAAGLTNIGIKKGDVIMTFLQNCAEFVFTFLSASMIGAVITTANPFYTKAEAFKQLKASNAKLIVTQSQYVDKFRDSGENDPKIGEDFSVITIDDPPENCLHFSVLSEANEEEMPKGIVIQPDDPVALPFSSGTTGLPKGVILTHKSLITGVAQLVDGDNPNLYLKQDDVVLCVLPLFHIFALNSVLLVSLRAGASVLLMQKFEIGALLELIQNHRVSVAAVVPPLVLALAKNPMVDSFDLSSIRLVLSGAAPLGKELEEALHQRVPQAIFGQGYGMTEAGPVVTMCPAFAKQPFSTKSGSCGSVVRNADLKVVDPETGGSLGRNQPGEICIRGSQIMKGYLNDDEATARTIDVDGWLHTGDIGYVDDDDEIYIVDRVKELIKFKGFQVPPAELESLLVSHPDIADAAVVPQKDDAAGEVPVAFVVRSANGFEITEEAIKEFIAKQVIFYKRLHKVYFIHAIPKSPSGKILRKELRAKLAAPSTQ

SEQ ID NO: 4 - protein, 551 aa, Nicotiana sp.
MGTRAVESSQQQECEHIFRSRYPPVQVPDNVTLPDFVLHNVELYTDKMAFVDATTGKGYTYGQVARDIRRFAKALRSLGLRKGRVVVVVLPNVPEYAIVALGIMAAGGVFSGANPAAHSSEIVKQVESADGKLIVSDLPTYHKVKDCGLPVIILGEEHVEGTIHWDELLEAAERAGSRTDHITNHEDEMVQQNDLCALPFSSGTTGLSKGVMLTHRNLVANLCSTLFSVSPEMVGQVTTLGLIPFFHIYGITGICCATIRNKGKVVVLRRYELRAFLNALITHEVTFAPIVPPIILALVKNPIVDEFDLSKLKLRSIMTAAAPLAPEILNEFEKKFPDVQVQEAYGMTEHSCITLSHSDQHTAKRNSVGFILPNLEVKFVDPDTGRSLPKNKPGEICVKSQCVMKGYYKNEFETCLTIDKDGWLQTGDIGYIDDDGDIFLVDRIKELIKYKGFQVAPAELEGILLTHPSVEDAAVVGLPDEEAGEIPVAWVVLNSKAKESEEDIINYIASTVAQYKRVRVVQFVDSIPKSPSGKILRRLIKDKMLERLKNA

SEQ ID NO: 5 - protein, 671 aa, Nicotiana sp.
MTEIPPNSQMKTMLQKAVQSVQWTYTLFWQLCPQQGALVWRDGYYNGAIKTRKTVQPMEVSAEEASLHRSQQLRELYESLSAGESNQPARRPSAALSPEDLTESEWFYLMCVSFSFPPGIGLPGKAYSKKHHIWIMGANEVDSKVFCRAILAKSARIQTVVGIPLLDGVLELGTTERVQEEIGFINHVKSFFTEQQQPQLPKPALSEHSTSNPTTFSEPHFYSGNTSPSANVDIAHQDGGAAGEEDEEEEEEEDDDEAELDSDSIAIQSAANPIAVEASELMQLDVSEAIQLGSPDDDSDNMDSDFHLVGAGNTAHDYQRQADSFKAETAISWPHFQDLQQLPGGSSYDELSQEDTHYSQTVSTILEHRSSKFSSTTMGCISHDSAQSAFTLCPSTTVCSPNPAHCRHDDSLVDGGGASQWLLKSILFTVPFLHTKYQSEASPKSRDVATVDSSSTASRFRKGCSITSQEEPSGNHVLAERRRREKLNERFIILRSLVPFVTKMDKASILGDTIEYVKQLRKKVQDLEARARDTEHSRDADKKGGTATVKVLQGRGKRRMNTVDGSVGGGQATITASPPSTTENEEVVQVQVSIIESDALVELRCPYKEGLLLNVMQMLRELKVEVVAIQSALNNGVFLAELRAKVKENICGRKASILEVKRSIHQIIPRD

SEQ ID NO: 6 - protein, 683 aa, Nicotiana sp.
MTEIPPNSQMQTMLQKAVQSVQWTYTLFWQLCSQQGVLVWRDGYYNGAIKTRKTVQPMEVSAEEASLHRSQQLRELYESLSAGESNQPARRPSAALSPEDLTESEWFYLMCVSFSFPPGIGLPGKAYSKKHHIWIMCANEVDSKVFCRAILAKSARIQTVVCIPLLDGVLELGTTERVQEDIGFINHVKSFFTEQQQPQPPKPALSEHSTSNSTTFSEPHFYSGNTPPSGNADIAQQDGGAAGEEDEEEEEEEDDEAELDSDSIAIQSEVGGAANPIAAEASELMQLDMSEAIRLGSPDDGSNNMDSDFHLVGAGNTADYQRQPDSFKAETAISWAHFQDLQHLPGGSSYEELSQEDTHYSQTVSTILEHFSNRSSKFSSTTMGCISHDSAQSAFTLCPSTTVDCSPNPAHCRRRHDDSLLDGGGASPSSQWLLKSILFTVPFLHTKYQSEASPKSVDVATVDSSSTASRFRKGCSITSQEEPSGNHVLAERRRREKLNERFIILRSLVPFVTKMDKASILGDTIEYVKQLHKKVQDLEARARHTEQSKDADQKSGTATVKVLQGRGKRRMNTVEAGNIGGGQAKMTAFPLSTTEDEEVVQVEVSIIESDALLELRCPYKEGLLLDVMQMLRELKVEVVAIQSSLNNGIFLAELRAKVKENIYGRKASIVEVKKSIHQIIPRD

SEQ ID NO: 7 - protein, 220 aa, Nicotiana sp.
MNICTNKSSSGVKKGAWTEEEDVLLKKCIEKYGEGKWHQVPLRAGLNRCRKSCRLRWLNYLRPHIKRGDFSFDEVDLILRLHKLLGNRWSLIAGRLPGRTANDVKNYWNSHLRKKLIAPHDQKESKQKAKKITIFRPRPRTFSKTNTCVKSNTNTVDKDIEGSSEIIRFNDNLKPTTEELTDDGIQWWADLLANNYNNNGIEEADNSSPTLLHEEMPLLS

SEQ ID NO: 8 - protein, 419 aa, Nicotiana sp.
MVVISAVVPTPSRVESLAKSGIQAIPKEYVRPQEELNGIGNIFEEEKKDEGPQVPTIDLKEIDSEDKEIREKCHKELKKAAMEWGVMYLVNHGISDQLIDRVKVAGKTFFDQPVEEKEKYANDQPSGNVQGYGSKLANSACGQLEWEDYFFHCVFPEDKCDLSIWPKIPTDYIPATSEYAKQIRNLATKILAVLSIGLGLEEGRLEKEVGGKEDLLLQMKINYYPKCPQPELALGVEAHTDVSALTFILHNMVPGLQLFYEGQWVTAKCVPNSIIMHIGDTLEILSNGKYKSILHRGVVNKEKVRISWAIFCEPPKEKIILKPLSETITEAEPPRFPPRTFAQHMAHKLFKKDDQDAAAEHKVSKKDDPDSAAGHKPFKKDDQDAVVQQKVLKEDEQDAAAEHKVFKKDNQDAAAEESK

SEQ ID NO: 9 - protein, 419 aa, Nicotiana sp.
MVVISAVVPTPSRVESLAKSGIQAIPKEYVRPQEELNGIGNIFEEEKKDEGPQVPTIDLTEIDSEDKEIREKCHQELKKAAIEWGVMHLVNHGISDELIDRVKVSGDTFFDQPVEEKEKYANDQPSGNVQGYGSKLANSACGQLEWEDYFFHCVFPEDKCNLSIWPKTPTDYIPATSEYAKQIRNLATKILAVLSIGLRLEEGRLEKEVGGMEDLLLQMKINYYPKCPQPELALGVEAHTDVSALTFILHNMVPGLQLFYEGQWVTAKCVPNSIIMHIGDTLEILSNGKYKSILHRGVVNKEKIRISWAIFCEPPKEKIILKPLPETITEAEPPRFPPRTFAQHMAHKLFKKDDQDAAAEHKVSKKDDPDSAAEHKPFKKDDQDAVAQQKVLKEDEQNAAAEHKVFKKDNQDAAAEESK

SEQ ID NO: 10 - protein, 622 aa, Nicotiana sp.
MAMGHQDQDGVPNNLRKQLALAVRGIQWSYAIFWSTPVTQPGVLEWSDGYYNGDIKTRKTVQVGEVNEDQLGLHRTEQLRELYSSLLTGEGEEDLQPQAKRPSAALSPEDLTDTEWYFLVCMSFVFNVGQGLPGKTSATNQTIWLCNAHQAESRVFSRSLLAKSASIQTVVCFPYLGGVIELGVTELVLEDPNLIQQIKNSFEVDHSVISKRPNYNSNDAKDDMNVASRKLDHNVLESDAYPVEINNSSPHDSSNGFVANQEAEDSLMVVGVIGETSQAQSWKFVDDNMSNGVHNSLNSSDCISQNYEKLSPLSNGEKETKPCPIDRQEHNQNKLHLLDHQGDDAQYQAVISTLLKSSDQLTLGPHFRNINKKSSFAGWKNDTEAPRIGTAQKLLKKVLLEVPRMHGGVTHKFSRENRKKNGLWRPEVDDIDRSRVISERRRREKINERFMHLASMLPTGGKVDKISLLDETIEYMKELERRVQELEARSGKKTNDTAEQTSDNCGTSKFNDVNGSLKRKACDMDEMEPESCNELLKGSSADGIVISMIDKEVSIKMRCLWSEGLLLKIMEALTDLQMDCHTVQSSKIDGILSIAIESKSNGLKTVSVGAIREVLQRVVWKS

SEQ ID NO: 11 - protein, 364 aa, Nicotiana sp.
MESSTKSQIPTQSEEERNCTYAMQLLSSSVLPFVLHSTIQLEVFEILAKSNDTKLSASQIVSQIPNCTKPEAPTMLNRMLYVLASYSLFTCSIVEDEKNNGGQKRVYGLSQVGKFFVKNENGASMGPLLALLQNKVFINSWFELKDAVLEGGVPFDRVHGVHAFEYPKSDPKFNDVFNKAMINHTTVVMKKILENYKGFENLKTLVDVGGGLGVNLKMITSKYPTIKGTNFDLPHVVQHAPSYPGVEHVGGDMFESVPEGDAIFMKWILHDWSDSHNLKLLKNCYKALPDNGKVIVVEAILPVKPDIDTAVVGVSQCDLIMMAQNPGGKERSEEEFRALATEAGFKGVNLICCVCNFWVMEFCK

SEQ ID NO: 12 - protein, 506 aa, Nicotiana sp.
MDLLLLEKTLIGLFFAIIVAIVVSKLRSKNFKLPPGPIPVPVFGNWLQVGDDLNHRNLTEYAKKFGDMFLLRMGQRNLVVVSSPELAKEVLHTQGVEFGSRTRNVVFDIFTGKGQDMVFTVYGEHWRKMRRIMTVPFFTNKVVQQYRRGWEDEVAHVVEDVKKNPESATNGIVLRKRLQLMMYNNMYRIMFDRRFESEDDPLFNKLKALNGERSRLAQSFEYNYGDFIPILRPFLRGYLNICKEIKQRRLQLFKDYFVDERKKLANTTKSMDNNSLKCAIDHILEAEQKGEINEDNVLYIVENINVAAIETTLWSIEWGIAELVNHPEIQKKLRDEIDSVLGVGVQITEPELNKLPYLQAVIKETLRLRMAIPLLVPHMNLHDAKLAGYDIPAESKILVNAWWLANNPATWKKPEEFRPERFFEEEKHVEANGNDFRYLPFGVGRRSCPGIILALPILGITLGRLVQNFELLPPPGQSKLDTTEKGGQFSLHILKHSTIVMKPRSF

SEQ ID NO: 13 - protein, 298 aa, Nicotiana sp.
MGRKPCCVKEGLRKGPWSSKEDLLLTNYIKENGEGQWRSLPKNAGLLRCGKSCRLRWMNYLRPGIKRGNFSQDEEDLIVRLHSLLGNRWSLIAGRLPGRTDNEIKNYWNTHLIKKLKNAGIEPKPHKNFSKCSKKESRKGPQQGKSRKIQGKKSNNKNNKGQIVQVEKTKVFFPKPIRISCGISRNNSFENVTLSTTCSSNSNSGEANLENKENEVKLEEVSFFPRDLDFGKLLEGDAIYDEFLMEESCHISNKCSLPMNESMLEKVYEEYLLLLSENCYLQDDHQNEQNFPVNVSDQ

SEQ ID NO: 14 - protein, 381 aa, Nicotiana sp.
MASEAHAAVHAPPPVAPTVCVTGAAGFIGSWLVMRLLERGYNVHATVRDPENKKKVKHLFELPKADTNLTLWKADLSVEGSFDEAIQGCQGVFHVATPMDFESEDPENEVIKPTVRGMLSIIESCAKANTVKRLVFTSSAGTLDAQEHQKLFYDETSWSDLDFIYAKKMTGWMYFVSKILAEKAAMEAAKKKNFDFISIIPPLVVGPFLTPTFPPSLITALSLITGNEAHYCIIKQGQYVHLDDLCEAHIFLYEQPKAEGRFICASHHAIIYDVAKMVREKWPEYYVPTEFKGIDKDLPVVYFSPKKLTDMGFQFKYTLEDMYKGAIETCRQKQLLPFSTQSTADNGRDKETIPLSAENYASGKENSPVANGTGKSTNGEI

SEQ ID NO: 15 - protein, 382 aa, Nicotiana sp.
MASEGHAAVHAPSPPAAPTVCVTGAAGFIGSWLVMRLLERGYNVHATVRDPENKKKVKHLLELPKADTNLTLWKADLSVEGSFDEAIQGCQGVFHVATPMDFESEDPENEVIKPTVRGMLSIIESCAKANTVKRLVFTSSAGTVDVQEHQKLLYDETSWSDLDFIYAKKMTGWMYFVSKILAEKAAMEAAKKKNIDFISIIPPLVVGPFLAPTFPPSLITALSLITGNEAHYSIIKQGKYVHLDDLCEAHIFLYEHPKAEGRFICASHHAIIYDVAKMVQEKWPEYYVPTEFKGIDKDLSVVYFSSKKLTDMGFQFKYTLEDMYKGAIETCRQKQLLPFSTRSTADNVRDKEAIPLSTENYASGKENSPVANGTGKSTNGEI

SEQ ID NO: 16 - protein, 213 aa, Nicotiana sp.
MGRKPCCSKEGLRKGTWTAKEDMLLTNYINEHGEVGWRSLPMKAGSRWSLIAGRIPGRTDNEIKNYWNTHLLKKLKSEGLEPKIHKSLAKNTRRQKEKANVSSQINQKGYKEKKKRNKKGNIEENCNNIEEKEQVAKKIEEQWHTQDSVQAMSGFSSTSEVASEKETNCNNVHCPSSGQSLEENDNEIYEKLQASGDSKRCKLNFSAEVNKTP

SEQ ID NO: 17 - protein, 435 aa, Nicotiana sp.
MKIEVKESTMVKPAAETPQQRLWNSNVDLVVPNFHTPSVYFYRPTGSPNFFDGKVLKEALSKALVPFYPMAGRLCRDEDGRIEIDCKGQGVLFVEAESDGVVDDFGDFAPTLELRQLIPAVDYSQGIQSYALLVLQITHFKCGGVSLGVGMQHHAADGASGLHFINTWSDMARGLDLTIPPFIDRTLLRARDPPQPQFPHVEYQPPPTLKVTPENTPISEAVPETSVSIFKLTRDQINTLKAKSKEDGNTVNYSSYEMLAGHVWRSTCMARGLAHDQETKLYIATDGRSRLRPSLPPGYFGNVIFTTTPIAVAGDIQSKPIWYAASKLHDALARMDNDYLRSALDYLELQPDLKALVRGAHTFKCPNLGITSWSRLPIHDADFGWGRPIFMGPGGIAYEGLSFILPSPTNDGSQSVAISLQAEHMKLFEKFLYDF

SEQ ID NO: 18 - protein, 436 aa, Nicotiana sp.
MGSEKMMKINIKESTLVKPSKPTPTKRLWSSNLDLIVGRIHLLTVYFYKPNGSSNFFDSKIMKEALSNVLVSFYPMAGRLARDEQGRIEINCNGEGVLFVEAESDAFVDDFGDFTPSLELRKLIPTVDTSGDISTFPLIIFQVTRFKCGGVSLGGGVFHTLSDGLSSIHFINTWSDIARGLSVAIPPFIDRTLLRARDPPTSSFEHVEYHPPPSLISSSKSLESTSPKPSTTTMLKFSSDQLGLLKSKSKHDGSTYEILAAHIWRCTCKARALSDDQLTKLHVATDGRSRLCPPLPPGYLGNVVFTGTPMAKSSELLQEPLTNSAKRIHSALSKMDDNYLRSALDYLELLPDLSALIRGPTYFASPNLNINSWTRLPVHDSDFGWGRPIHMGPACILYEGTVYILPSPNSKDRNLRLAVCLDADHMPLFEKYLYEF

SEQ ID NO: 19 - protein, 715 aa, Nicotiana sp.
MASNGHVNGGENFELCKKSADPLNWEMAAESLRGSHLDEVKKMVSEFRKPMVKLGGESLTVAQVAAIAVRDKSANGVKVELSEEARAGVKASSDWVMDSMNKGTDSYGVTTGFGATSHRRTKNGGALQKELIRFLNAGVFGNGTETSHTLPHSATRAAMLVRINTLLQGYSGIRFEILEAITKLINSNITPCLPLRGTITASGDLVPLSYIAGLLTGRPNSKAVGPNGETLNAEEAFRVAGVNGGFFELQPKEGLALVNGTAVGSGMASMVLFDSNILAVMSEVLSAIFAEVMNGKPEFTDHLTHKLKHHPGQIEAAAIMEHILDGSSYVKAAQKLHEMDPLQKPKQDRYALRTSPQWLGPQIEVIRAATKMIEREINSVNDNPLIDVSRNKALHGGNFQGTPIGVSMDNARLALASIGKLMFAQFSELVNDYYNNGLPSNLTASRNPSLDYGFKGAEIAMASYCSELQFLANPVTNHVQSAEQHNQDVNSLGLISARKTAEAVDILKLMSSTYLVALCQAIDLRHLEENLKNAVKNTVSQVAKRTLTMGANGELHPARFCEKELLRIVDREYLFAYADDPCSCNYPLMQKLRQVLVDHAMNNGESEKNVNSSIFQKIGAFEDELKAVLPKEVESARAALESGNPAIPNRITECRSYPLYRFVRKELGTELLTGEKVRSPGEECDKVFTAMCNGQIIDPMLECLKSWNGAPLPIC

SEQ ID NO: 20 - protein, 341 aa, Nicotiana sp.
MDNSAPDSLSRSETAVTYDSPYPLYAMAFSSLRSSSGHRIAVGSFLEDYNNRIDILSFDSDSMTVKPLPNLSFEHPYPPTKLMFSPPSLRRPSSGDLLASSGDFLRLWEINEDSSTVEPISVLNNSKTSEFCAPLTSFDWNDVEPKRLGTCSIDTTCTIWDIEKSVVETQLIAHDKEVHDIAWGEARVFASVSADGSVRIFDLRDKEHSTIIYESPQPDTPLLRLAWNKQDLRYMATILMDSNKVVILDIRSPTMPVAELERHQASVNAIAWAPQSCKHICSGGDDTQALIWELPTVAGPNGIDPMSVYSAGSEINQLQWSSSQPDWIGIAFANKMQLLRV

SEQ ID NO: 21 - protein, 440 aa, Nicotiana sp.
MSMSVALLWVVSPTSEVSNGTGLLDSVREGNRVFVSSRFLARDRNLMWNGRIKKGGRQRWNFGSLIADPRYSCLGGSRTEKGSSFSVQSSLVASPAGEMTVSSEKKVYDVVLKQAALVKRQLRSTDELEVKPDIVVPGNLGLLSEAYDRCGEVCAEYAKTFYLGTKLMTPERRRAIWSIYVWCRRTDELVDGPNASHITPQALDRWEARLEDIFSGRPFDMLDAALSDTVSRFPVDIQPFRDMIEGMRMDLWKSRYNNFDELYLYCYYVAGTVGLMSVPVMGIAPESKATTESVYNAALALGLANQLTNILRDVGEDARRGRVYLPQDELAQAGLSDEDIFAGRVTDKWRNFMKKQIQRARKFFDESEKGVTELDSASRWPVSTALLLYRKILDEIEANDYNNFTRRAYVSKPKKLLTLPIAYAKSLVPPNRTSSPLAKT

SEQ ID NO: 22 - protein, 440 aa, Nicotiana sp.
MSMSVALLWVVSPTSEVSNGTGLLDSVREGNRVFVSSRFLARDRNLMWNGRIKKGGRQRWNFGSLIADPRYSCLGGSRTEKGSSFSVQSSLVASPAGEMTVSSEKKVYDVVLKQAALVKRQLRSTDELEVKPDIVVPGNLGLLSEAYDRCGEVCAEYAKTFYLGTKLMTPERRRAIWAIYVWCRRTDELVDGPNASHITPQALDRWEARLEDIFSGRPFDMLDAALSDTVSRFPVDIQPFRDMIEGMRMDLWKSRYNNFDELYLYCYYVAGTVGLMSVPVMGIAPESKATTESVYNAALALGLANQLTNILRDVGEDARRGRVYLPQDELAQAGLSDEDIFAGRVTDKWRNFMKKQIQRARKFFDESEKGVTELDSASRWPVLAALLLYRKILDEIEANDYNNFTRRAYVSKPKKLLTLPIAYAKSLVPPNRTSSPLAKT

SEQ ID NO: 23 - protein, 248 aa, Nicotiana sp.
MEGSSKGLRKGAWTTEEDSLLRQCINKYGEGKWHQVPVRAGLNRCRKSCRLRWLNYLKPSIKRGKLSSDEVDLLLRLHRLLGNRWSLIAGRLPGRTANDVKNYWNTHLSKKHEPCCKIKMKKRDITPIPTTPALKNNVYKPRPRSFTVNNDCNHLNAPPKVDVNPPCLGLNINNVCDNSIIYNKDKKKDQLVNNLIDGDNMWLEKFLEESQEVDILVPEATTTEKGDTLAFDVDQLWSLFDGETVKFD

SEQ ID NO: 24 - DNA, 992 bp, Nicotiana sp.
  source 1..992: mol_type = genomic DNA
atggaaagcaagagttccggaggcggagaaggaaaggttgtatgtgtaacaggggcctctggtttcatagcttcatggcttgttaagatgctacttcaacgtggttacactgtcaatgccactgttcgcaacctcaaggatgcgagtaaagtggatcacctgttaggccttgacggagctaaagagaggctgcatcttttcaaagctgagttacttggtgagcattcgtttgatcctgccgttgatggttgtgaaggtgtctttcatacagcatcacctgtttctctcacagctaaatccaaggaggaacttgtagaccctgctgtgagcggaacattaaacgtccttaggtcatgtaccaaatcaacatctgttagaagagtggtcataacctcttctaccgcttctgttatttgcaataaaaacatgtcaacccctggagctgtagctgatgagacttggtattcagatgcagaactctgtgaggaaagaaaggaatggtatcaactctccaaaaccttggctgaggaagctgcttggaaatttgcaaaggagaatgggttggacttggttacacttcatccaggtctagtcatcggtccacttctgcagcctacgctcaatttctcgtgcgaggctatagtgaacttcataaaagaaggaaaagaagcatggtctggcggaatatatagatttgtcgatgttagggatgttgctaatgcacatatactagcatttgaggtcccttcagcaaatggaagatattgtttagttggggtaaatggatattcttctttggttttgaagattgtacaaaagctttacccttccatcactctccctgagaattttgaagatggattacctcttatcccaaccttccaagtatcaagcgaaagagcaaaaagtttaggcgtcaatttcacatctcttgagttgagcgtgaaggacactgttgaaagcttgatagagaagaacttcctcaagatttg

SEQ ID NO: 25 - DNA, 1251 bp, Nicotiana sp.
  source 1..1251: mol_type = genomic DNA
atgatgtgcaatattatttcacttgtgtccatcatcaacttctttttgctgttttatagagttgttgtcctaccacaacatgctgctgcttctcactctaccgttgaatttcttcctggatttgaaggtccacttcctttccatcttgagactgggtatattggagtaggtgaatatgaagaagtgcagctcttttattattttcttaaatcagaatcagaacccacaaaagatcctattttgatttggctctcaggaggacctggttgctcttcctttactgcacttgtttatcaaatagggcccttgtattttgagccaaacgagtataatgggagccttccaaagctaacattgaatccaaactcatggaccaaggtagctaacataatattcttggatcaacctgtgaatagtggcttctcttatgcaacaacttcaacaacattcaagtctactgatctacaagcatgccaccatatctaccagtttttgcgaaagtggttgattaagcatcaagagttcattaggaatccaatgtacattggtggagattcatattctggcatcactgttccagttatcactcaactaatatcaaatggaattgaagcagggcacaagccatcgattaatcttaagggatatatacttgggaatcctagcacgtttcctcttcaatataactactgggttccttatgctcatggaatgggacttatctccgacgaactttatcaggctactactcttatcctttactttaaaatttatcatatcataaatgcaattaatttagcttacatggttgaggtacacaggttgtctacttactgggcaaatgatccaagagtacaagaagctcttaatgttcgtaagggagctataacaagatggacaagatgtagggaaagtatcgtgaataaaacttacactattactttccaagatagcataccttatcatgtggaactcagcaaaaaactttatcgatcacttatatacagtggcgatcatgatatgggcattccattccaatcaactcaattttggataaaatctctaaattattctattgtggatgaatggcggccatggagttttgatggtcaagttgcaggatatacaagatcctattccaaccagatgacatttgcaactgtcaagggagcaggacatgtagctcctgagtacaagcctaaagagtgctttaccatgtttcaaagatggttgtctcatgaaccactttga

SEQ ID NO: 26 - DNA, 1710 bp, Nicotiana sp.
  source 1..1710: mol_type = genomic DNA
atgttgagtgtagctagtgttgaagcccagaaagcagagttatcttcttctgtaattcctccttctgatcaatctactgaagagatacacgtgtttagatcaagattaccagatatacaaatttccaacaatgttcctcttcatgtttacttgtttgagaggctctctgaatttcaagataggacatgtcttatagcaggcagcagtggacaatcctacacttttgctgaaactcatctcatttgccagaaaatagctgctggcttaacaaatataggaatcaaaaagggagatgtaatcatgacttttctccagaactgcgcggaatttgtgtttacttttctctcggcttctatgattggcgccgttataactacagctaatccattttacacaaaagcagaggcgtttaagcaattaaaagcgtcgaatgcaaaactaatcgttactcaatctcagtacgtggataaatttcgtgattctggagagaatgacccgaaaattggcgaagatttttcagtcattacaattgatgacccccctgaaaattgcttacatttctctgtactttctgaagctaacgaagaggaaatgccaaagggaattgtaatccaaccagatgatccagtagctttaccattttcttcaggaacaacagggctaccaaaaggtgtgattttaactcacaaaagtttaatcacaggagtagctcaattagtcgacggagataatccaaatttgtacttaaaacaagacgacgtggtgctatgtgtgctacctttgtttcacatatttgcgttaaattcagtgcttttagtctcgttaagagcaggagctagtgttttactaatgcaaaaattcgaaattggtgcattgctggagctgatacaaaaccaccgcgtgtcagttgcagcagtagttccgcccttggttcttgcgttggcgaaaaatccgatggttgattcgttcgatttgagttcgattaggctcgtgttgtccggggcggcgccgctggggaaagagttggaggaagcgctacatcaaagagtcccgcaagctatatttggtcaggggtatggtatgacagaggcaggaccagtagtaacaatgtgcccagcatttgcaaagcaaccattttcaaccaaatctggctcatgtggttcagtagttcgaaatgcagacctcaaggtggtcgaccccgaaactggtggctccctcggccgcaaccaacccggcgaaatttgcatccgtggttcccaaatcatgaaaggttatctgaatgatgatgaggccacggcacggaccatagatgtcgatggatggctccacactggtgatataggatacgtagatgacgatgatgaaatatacatcgtcgatagagtcaaagagcttatcaagttcaaaggattccaggtgccaccagctgagcttgagtcccttctagtaagccatccagatattgcagatgctgctgttgtaccgcaaaaagatgatgcggcaggggaagtcccagttgcatttgtggtccgctccgccaatggttttgaaattactgaagaagctataaaggaatttattgccaaacaggtgatattctataaaagattgcacaaggtgtattttattcacgctattccaaagtctccgtctggaaagatactgaggaaagaactgagagccaaactagctgcgccctccacccagtga

SEQ ID NO: 27 - DNA, 1656 bp, Nicotiana sp.
  source 1..1656: mol_type = genomic DNA
atgggaactcgtgcagtagaaagctcacagcagcaagaatgtgaacatattttccggagtagatatcctccggttcaagtaccggacaatgtgaccctcccggattttgtgcttcacaatgtagagttatacactgacaaaatggcatttgtggatgctaccactggcaaaggctacacttatggccaagttgcaagagacataaggaggttcgccaaggccttgagatcccttggcttaaggaaaggacgggtggtggtggtagttcttccaaatgtaccagaatatgctattgttgctcttggaatcatggctgctggtggcgtcttctccggtgcaaatccagcagctcattcatcagaaatcgtgaaacaagttgaatctgctgatggcaagcttattgtctctgatctaccaacctatcacaaggttaaagattgtgggctgccagtaataatactaggtgaagaacatgtagaaggaacaattcattgggatgaattgcttgaagctgcagagcgtgccggttccagaactgatcacataacaaaccatgaagatgaaatggtgcagcaaaatgatttatgtgcactgcccttctcgtcaggcactacggggctgtccaagggagtgatgttaacccacagaaatctagtagcaaacctctgctctacactcttcagtgttagcccagaaatggtaggccaagttacaacactgggtttgataccattcttccacatttatgggataactggaatctgttgtgcaaccattagaaacaaagggaaagtggtagtcttgcgtaggtacgaactgagggcatttctaaatgcactcattacacatgaagtcacatttgcaccaattgtgccacctatcatcttggcacttgttaagaatcctattgtggatgaatttgatctcagcaagcttaagcttagatccatcatgacagcggcagccccacttgcccctgagattcttaatgaatttgaaaagaaatttcccgatgttcaggtccaagaggcatatgggatgactgagcacagctgcattactctttctcatagtgaccagcatactgctaaaagaaattctgttggttttattctacctaatttggaggtaaagttcgttgatcctgataccggtagatctctccccaaaaacaaaccaggcgagatatgtgtcaaaagccaatgtgttatgaagggttactacaaaaatgaatttgagacttgccttaccattgataaggatggatggcttcagactggtgacattggctacattgacgatgatggagatatcttcctagtcgatcgtatcaaagagcttatcaagtacaagggattccaagttgctccagctgagttagaagggatccttctcacacatccttcagtagaagatgctgcagtagttgggctgccagatgaagaagcaggagagataccagtggcatgggtagtcttgaactcaaaagcaaaagaaagcgaagaggacattatcaactacattgcatcgactgttgcacagtataaacgagtgagagtggtgcagttcgttgatagtattccaaaatctccttctggaaaaatactgagaagacttatcaaggataagatgctagagagacttaagaatgcatag

SEQ ID NO: 28 - DNA, 2016 bp, Nicotiana sp.
  source 1..2016: mol_type = genomic DNA
atgacggagataccgcctaacagccagatgaaaaccatgttgcagaaggcagtgcaatcggttcaatggacatatactcttttctggcaattatgtccccaacaaggggcgttagtgtggagagatggatattacaatggggctataaagactagaaagacagtgcagccaatggaagttagcgctgaggaagcttctcttcacagaagccaacagcttagagaactttacgaatcactttccgccggcgagtcaaatcagccagcgagaaggccgtcggcagctttgtcaccggaggacttgacggagtccgagtggttttatctcatgtgtgtttctttctcttttcctcctggcatcggattacctggcaaggcttattcgaagaaacatcacatatggatcatgggcgcaaacgaggttgatagcaaagtcttctgtagagctattcttgccaagagcgcccgcatacagacggtcgttggtattcctctcttggatggtgtactggaactgggaactacagaaagggttcaagaagagattggattcataaaccatgtaaagagctttttcactgagcaacaacaacctcagctaccaaagccagccttatctgagcactccacttccaatcccaccaccttttccgagccacatttttactccggcaatacttcgccatctgctaatgttgatattgcgcatcaagatggcggagctgccggcgaagaagatgaggaggaggaagaagaagaagatgatgatgaagccgagttggactcggatagtatagcgattcaaagcgcggctaatcctattgccgttgaggctagtgaactcatgcagcttgatgtgtccgaggctatacagctcggctcgcccgatgatgactctgataatatggactctgattttcatttggttggcgctggaaacacggctcatgactaccagcgccaagctgactctttcaaagccgagaccgccattagctggccgcacttccaagaccttcaacaattaccaggtggctctagttatgatgaattatcacaagaagacacacactattctcaaacagtgtcaaccattctcgaacaccgaagctccaaattttcctctacaacaatgggctgtatttctcatgactcggcccaatctgccttcacattgtgccctagcaccaccgtctgcagcccgaatcccgcccactgccgccacgacgactcacttgtcgacggtggcggcgcctcccagtggctgctcaaaagcatactcttcactgtcccatttcttcacactaaataccaatctgaagcttctccaaagtcacgtgacgtcgccactgttgattcctccagtactgcttctcgctttcgcaaaggctgtagtataacgtcgcaagaagagccaagtggaaaccatgtacttgcagaacgacgtcgtagagagaagctaaatgagcgttttatcatattaaggtctcttgtaccttttgtaacgaaaatggacaaagcctccattttgggtgacaccatagagtatgtcaagcagttacgtaagaaagttcaggatcttgaagctcgtgctcgcgacacggagcactccagagatgcagataaaaaaggtggcacagctacagtgaaggtgttgcaaggaaggggtaagaggagaatgaatacggtagatggaagtgttggtggagggcaggcaacgataacggcgtccccaccgtcaacgacggaaaatgaggaggttgtgcaagtacaagtatcaattatcgaaagcgatgcattggtggagctccggtgtccgtacaaagaggggttgctgttaaatgtaatgcagatgctaagggaactcaaagtggaagttgtagccattcaatcagctcttaataatggcgtcttcttggctgagttaagagctaaggtaaaagagaatatatgtggaaggaaagcaagcattttggaagtaaaaaggtcaatacatcagataatccctagagattaa

SEQ ID NO: 29 - DNA, 2052 bp, Nicotiana sp.
  source 1..2052: mol_type = genomic DNA
atgacggagataccgcctaacagccagatgcaaaccatgttgcagaaggctgtgcaatcggttcaatggacatatactcttttctggcaattatgttcccaacaaggggtgttagtgtggagagatggatattacaatggggctataaagactagaaagactgtgcagccaatggaagttagcgctgaggaagcttctcttcacagaagccaacagcttagagaactttacgaatcactttccgccggcgagtcaaatcagccggcgagaaggccgtcggcagctttgtcaccggaggacttgacggaatccgagtggttttatctcatgtgtgtttctttctcttttcctcctggcatcggattacctggcaaggcttattcaaagaaacatcacatatggattatgtgcgcaaacgaggttgatagcaaagtcttctgtagagctattcttgccaagagtgcccgcatacagacggtcgtctgtattcctctcttggatggtgtactggaactgggaactacagaaagggttcaagaagacattggattcataaaccatgtaaagagctttttcactgagcaacaacaacctcagccaccaaagccagccttatctgagcactccacttccaattccaccaccttttccgagccacacttttactccggcaatactccgccatctggcaatgctgatattgcgcagcaagatggcggagctgccggagaagaagatgaggaggaggaagaagaagaagacgatgaagccgagttggattcggatagtatagcaattcaaagtgaggttggtggcgcggctaatcctatagcggctgaggctagtgaactcatgcagcttgatatgtctgaggctatacggcttggctcgcccgatgatggctctaataatatggactctgattttcatttggttggcgctggaaatacggctgactaccagcgtcaacctgactctttcaaagccgagactgccattagctgggctcacttccaagaccttcaacatttaccaggtggctctagttatgaagaattatcacaagaagacacacattattctcaaacagtgtcaaccattcttgaacacttctcaaaccgaagctccaaattttcctctaccacaatgggctgtatttctcatgactcagcccaatctgccttcacattgtgccctagcaccaccgtcgactgcagcccgaatcccgcccactgccgccgccgccacgacgattcacttctcgacggtggcggcgcctccccctcctcccagtggctgctcaaaagcatactcttcactgtcccatttcttcacactaaataccaatctgaagcttctccgaaatcagttgacgtcgccactgttgattcctccagtactgcttctcgctttcgcaaaggctgtagtataacgtcgcaagaagagcccagtggaaaccatgtacttgcagaacgacgtcgtagagaaaagctaaatgagcgttttatcatattaaggtctcttgtaccttttgttacgaaaatggataaagcctccattttgggtgacaccatagagtatgtcaagcagttacataagaaagttcaggatcttgaagctcgtgctcgtcacacggagcagtccaaagatgcagaccaaaaaagtggcacagctacagtgaaggtgttgcaagggaggggtaagaggagaatgaatacggtggaggccggaaatattggtggagggcaggcaaagatgacggcttttccgctatcaacaacggaggatgaagaggttgtgcaagtagaagtatcaattattgaaagcgatgcattgttggagctccgatgtccgtacaaagaggggctgctgttagatgtaatgcagatgctaagggaactcaaggtggaagttgtagccattcaatcatctcttaataatggcatcttcttggctgagttaagagctaaggtaaaagagaatatatatggaaggaaagcaagcattgtggaagtaaaaaagtcaatacatcagataatccctagagattaa

SEQ ID NO: 30 - DNA, 663 bp, Nicotiana sp.
  source 1..663: mol_type = genomic DNA
atgaatatttgtactaataagtcgtcgtcaggagtgaagaaaggtgcatggactgaagaagaagatgttctattgaaaaaatgcatcgagaaatatggagaaggaaagtggcatcaagttcctcttagagctggtttgaatagatgcagaaagagctgcagattaaggtggctaaattatctaaggccacatataaagagaggagacttctcttttgatgaagtagatctcattttgaggcttcataagctgttaggcaacagatggtcacttattgctggtagacttcctggaaggacggcaaacgatgtcaaaaactactggaacagccatcttcgcaagaagttaattgctcctcatgatcaaaaggagagcaagcaaaaagcaaagaagatcaccatattcagacctcggcctcgaaccttctcaaagacaaatacttgtgttaaaagtaacacaaatactgtagataaggatattgaaggcagcagcgaaataattagattcaacgataatttgaagccaacaactgaagaattgacggatgatggaattcaatggtgggccgatttactagctaacaattacaacaataatgggattgaggaagctgataattcatcaccaactttgttgcatgaggaaatgccacttctcagttga

SEQ ID NO: 31 - DNA, 1260 bp, Nicotiana sp.
  source 1..1260: mol_type = genomic DNA
atggtggtgatcagtgcagtagttccaactccttcaagagttgaaagcttggctaaaagtggaatccaggctatccctaaagagtatgtgaggccacaagaagagttaaatggaataggaaacatatttgaggaagagaagaaagatgaaggacctcaagtaccgacgattgatctgaaagaaatcgactcagaggacaaggaaattcgcgagaaatgccacaaagagttgaagaaagcagctatggagtggggtgttatgtaccttgttaaccatggcatatcagatcagctaattgatcgtgtcaaggttgctggaaagaccttctttgatcaacctgttgaagaaaaggagaagtatgctaatgaccaaccctctggcaatgtccaaggctatggcagcaagttagcaaatagtgcttgtggtcagcttgaatgggaggattatttcttccattgcgttttccccgaggacaagtgcgacttatccatctggcctaaaatccctactgactacattccagcaacaagtgaatatgccaaacagattaggaacctagcaacaaagattttggcagtgctttctattgggctgggactagaagaaggaagactagagaaggaagtcggaggcaaggaggacctactgcttcaaatgaagattaactactaccccaaatgtccccaaccagaactagcacttggcgttgaagctcatactgatgtgagtgcactgacttttatcctccacaatatggtgcctgggttacaacttttctatgaaggacagtgggtaacggcaaagtgtgtgcctaattccataatcatgcatattggggacacccttgaaatcctaagcaatggaaagtacaaaagcattcttcacagaggggttgtgaataaagagaaagtaagaatctcatgggctattttctgtgagccgccaaaggagaagatcatccttaagcccctatctgagactatcactgaggctgaaccacctcgattcccacctcgcacctttgcacagcatatggcccataagctcttcaagaaggatgatcaggatgctgctgctgaacacaaagtctccaagaaggatgacccggattctgctgctggacacaaacccttcaagaaggatgatcaggatgctgttgttcagcaaaaagtcctcaaggaggatgaacaggatgccgctgctgagcacaaagtcttcaagaaggataatcaggatgctgctgctgaagaatctaaatag

SEQ ID NO: 32 - DNA, 1260 bp, Nicotiana sp.
  source 1..1260: mol_type = genomic DNA
atggtggtgatcagtgcagtagttccaactccttcaagagttgaaagcttggctaaaagtggaatccaggctatccctaaagagtatgtgaggccacaagaagagttaaatggaataggaaacatatttgaggaagagaagaaagatgaaggacctcaagtaccgacgattgatctaacagaaatcgactcagaggacaaggaaattcgagagaaatgccaccaagagttgaagaaagcagctatagaatggggtgttatgcaccttgttaaccatggcatatcagatgagctaattgatcgtgtcaaggtttctggagataccttctttgatcaacctgttgaagaaaaggagaagtatgctaatgaccaaccctctggcaatgtccaaggctatggcagcaagctagcaaatagtgcttgtggtcagcttgagtgggaggattatttcttccattgtgttttccctgaggacaagtgcaacttatccatctggccgaaaacccctacagactacattccagcaacaagtgaatatgccaagcagatcaggaacctagcaacaaagattttggcagtgctttctattgggctgagactagaagaaggaagactagagaaggaagtcggaggcatggaggacctgctgcttcaaatgaagattaactactatcccaaatgcccccaaccagaactagcacttggtgtcgaagctcatactgatgtcagtgcactgacttttatcctccacaatatggtgcctggcttgcaacttttctacgaaggacagtgggtaacggcaaagtgtgtgcctaattccataatcatgcatattggggacacccttgaaattctaagcaatggaaagtacaagagcattcttcacagaggggttgtgaataaagagaaaataagaatctcatgggctattttctgtgagccgccgaaggagaagatcatccttaagcccctacctgagactataactgaggctgagccacctcgattcccacctcgcacctttgcacagcatatggcccataagctcttcaagaaggatgatcaggatgctgctgctgaacacaaagtctccaagaaggatgacccggattctgctgctgaacacaaacccttcaagaaggatgatcaggatgctgttgctcagcaaaaagtcctcaaggaggatgaacagaatgccgctgctgagcacaaagtcttcaagaaggataatcaggatgctgctgctgaagaatctaaatag

SEQ ID NO: 33 - DNA, 1869 bp, Nicotiana sp.
  source 1..1869: mol_type = genomic DNA
atggctatgggacaccaagaccaagatggagttccaaacaacttgagaaagcaacttgctcttgctgttagaggtattcaatggagctatgcaatcttctggtcaactccagttacacagccaggggtgttggaatggagtgatgggtactataatggggatatcaagactaggaagacagttcaggtgggggaagttaatgaagaccaacttgggttgcacagaactgagcaattgcgagaactttatagttcactcttaacaggtgaaggtgaagaagacttacaacctcaggctaaaaggccctcagctgcattatctcctgaagatctcactgatacggagtggtatttcttagtatgcatgtctttcgtcttcaatgttggacaagggttgccagggaagacctcagcgacgaatcaaacaatctggctatgcaatgctcaccaagcagagagtagagtattttctcgctctctgctagcaaagagtgcatctatccagactgtcgtatgctttccatatttaggaggcgttattgagctgggagtcaccgagcttgtcttagaagatcccaacctcattcagcaaataaaaaattcctttgaggttgatcactctgttatttcgaagaggcctaattacaactccaatgatgcaaaagatgacatgaatgttgctagccgaaagcttgatcataatgtacttgaaagtgatgcttatccagttgaaataaacaacagttcaccgcatgatagttcaaacggttttgtggccaatcaagaggcagaagattctttaatggtggtaggcgttataggggaaacttcacaagctcaaagctggaagttcgtggatgataatatgagtaacggtgtgcataattctttgaattccagtgactgcatctctcaaaattatgaaaagttgtcccctctttcgaatggagaaaaagaaactaagccttgcccaatagaccgtcaagagcacaatcagaataaactgcatcttttagatcaccaaggagatgacgctcaatatcaagctgtcatttctacccttttaaaaagctctgaccaattaactttgggaccacattttagaaatattaacaaaaagtcaagctttgctggttggaagaatgatactgaagcgccaagaataggaactgcacaaaaactattgaagaaggtacttcttgaagttcctagaatgcatggtggtgttacacataaattcagcagagagaatcgtaaaaagaacggcctttggagaccggaggttgatgacattgatagaagccgtgttatttcagagagaaggcgaagagaaaagataaacgagagatttatgcatcttgcatcaatgctgccgactggtggcaaggttgacaaaatatcactacttgacgagacaatagaatacatgaaagagcttgagaggagagttcaagagctggaagctagatcaggaaaaaaaacaaatgatactgcagagcagacatctgataattgtggcactagtaaattcaatgacgtcaatggatcgttaaagaggaaagcatgtgatatggatgaaatggaacctgaaagctgtaatgaattactgaaaggcagttcagctgatggtattgtcatcagtatgatcgataaggaagtctcgatcaagatgaggtgtctttggagcgagggcttgttacttaagattatggaggcactaaccgacctacaaatggattgccatacggttcaatcttccaagattgatgggattttatccattgctattgaatcaaagtcaaatggattgaaaactgtatcagttggagcaattagagaagtacttcagcgagtagtctggaaatcttga

SEQ ID NO: 34 - DNA, 1095 bp, Nicotiana sp.
  source 1..1095: mol_type = genomic DNA
atggaatcctcaaccaaaagccaaataccaacacaatcagaagaagagcgtaactgcacatatgccatgcaactattgtcatcttcagtcctcccctttgtgttgcattcaacaattcaattggaagtttttgagatattagccaaatctaatgacactaaactttctgcttctcaaattgtttctcaaattcctaactgcacaaaacctgaagcacctactatgttaaataggatgctttatgtcttggctagttactccttgtttacttgttccattgttgaagatgaaaaaaataatgggggccaaaaaagagtgtatggtttgtcacaagtgggaaaattctttgttaaaaatgaaaatggtgcatcaatggggccacttttggctttgcttcaaaataaagtattcataaacagctggtttgaactaaaagatgcagttcttgaaggaggagttccatttgacagggtacacggtgtgcatgcatttgaatatccaaaatcggacccaaaattcaatgatgttttcaacaaggcaatgatcaatcacacaactgtagtcatgaaaaaaatacttgaaaattacaaaggttttgagaaccttaaaactttggttgatgttggaggtggtcttggagttaacctcaagatgattacatctaaataccccacaattaagggcactaattttgatttgccacatgttgttcaacatgccccttcctatcctggggtggaacatgttgggggagatatgtttgaaagtgttccagaaggagatgctatttttatgaagtggattcttcatgactggagtgatagtcacaacctcaagttgctaaagaactgctacaaggctctaccagacaatggaaaggtgattgttgttgaggccattttaccagtgaaaccagacattgacaccgcagtggttggcgtttcgcaatgtgatttgatcatgatggctcaaaatcctggaggcaaagagcgatcggaagaggagtttcgagccttggctactgaagctggattcaaaggcgttaacttaatatgttgtgtctgtaatttttgggtcatggaattctgcaagtag

SEQ ID NO: 35 - DNA, 1521 bp, Nicotiana sp.
  source 1..1521: mol_type = genomic DNA
atggatcttctccttctagagaagacccttatagggttattctttgctatcattgtagctatagttgtttctaagctacgtagcaagaattttaagttgcccccaggtccgattcctgtgccagtttttggcaattggcttcaagttggtgatgacttgaatcacagaaacctcactgaatatgccaagaaatttggtgacatgttcttgctaagaatgggacagaggaatcttgtggtggtgtcatcccctgaactagccaaagaagttttgcatacacaaggggttgaatttggatcaagaacaaggaatgtggtctttgatattttcactggaaaaggccaagatatggtttttacagtatatggtgaacactggaggaaaatgaggaggattatgactgttccattttttacaaacaaagtggtgcagcagtataggcgtgggtgggaagatgaagtggcacatgttgttgaggatgtgaagaaaaatccagagtcagcaactaatgggattgtgttgaggaaaaggttgcagcttatgatgtacaataacatgtacaggattatgtttgataggaggtttgagagtgaggatgatcctttgtttaacaagcttaaggctttgaatggggagaggagtagattggctcagagttttgagtacaattatggtgattttatccctatattgagacctttcttgagaggttacttgaacatctgtaaggaaattaagcagaggaggttgcagcttttcaaagattactttgttgatgaaagaaagaaacttgcaaacacgacgaagagcatggacaataattcgctaaagtgtgccattgatcacattcttgaggctgaacagaagggagagatcaatgaggataatgtcctttacattgttgagaacatcaatgttgctgcaatagaaactacactgtggtcaatcgagtggggtattgctgaactagtgaaccaccctgaaatccagaagaaactccgtgacgagattgacagtgttcttggagtaggagtgcaaatcactgagccagaactcaacaagcttccttaccttcaggctgtgatcaaggagacccttcgtctccgtatggcaatccctcttttagtcccacacatgaaccttcacgatgcgaagcttgctggatatgacattcccgcggagagcaagatcctggtaaacgcttggtggctggctaacaaccctgctacctggaagaagcccgaagagtttaggccagagaggttctttgaagaggagaagcacgttgaggctaatggcaatgacttcagatatcttccatttggtgttggtaggaggagctgccctggaattatccttgcactgccaattcttggcattaccttgggacgcttggtgcagaactttgagttgttgcctcctccaggacagtcaaagcttgacacaacagagaaaggcgggcaattcagtctgcacattttgaagcattccaccattgtgatgaaaccaagatctttttaa

SEQ ID NO: 36 - DNA, 897 bp, Nicotiana sp.
  source 1..897: mol_type = genomic DNA
atgggaagaaaaccatgttgtgtaaaagagggattgagaaaaggtccatggtcttctaaagaagatttattacttactaattatatcaaggaaaatggtgaaggacaatggagatctttgcctaagaatgctgggttgcttaggtgtggaaaaagttgtagactaagatggatgaactatttaagaccagggattaaaagaggaaatttcagtcaagatgaagaagatcttatagtgagactacattctcttttgggtaatcgttggtcactaattgctggaagattaccaggtcgtacagacaatgaaatcaagaattattggaacacacatttaatcaagaagctcaaaaatgctggaattgaaccaaaaccccacaaaaatttctccaaatgttccaaaaaggaatcaagaaaaggaccccaacaagggaaatcaagaaaaatacaaggcaaaaagagcaacaacaaaaacaataagggtcaaattgtacaagttgagaagaccaaagtatttttcccaaaacccatcaggatttcttgtggaatttcaaggaacaatagttttgaaaatgttacattgagtactacttgttcctcaaatagtaattctggagaagctaatcttgaaaacaaggaaaatgaggtgaaattagaagaagtttcattctttccaagggacttagattttggtaaattacttgaaggggatgcaatttatgatgaatttctaatggaagaaagttgccacatttcaaacaaatgttcattgccaatgaatgagagcatgttggagaaagtatatgaagaatatcttttacttctttctgaaaattgttaccttcaagatgatcatcaaaatgagcaaaatttccctgtaaatgtttctgatcagtga

SEQ ID NO: 37 - DNA, 1146 bp, Nicotiana sp.
  source 1..1146: mol_type = genomic DNA
atggcaagtgaagctcatgcagctgttcatgctcctcctccggtagcaccgacggtttgcgtcactggagcagctggatttattggctcttggcttgtcatgagactccttgaacgtggttataatgttcacgctactgttcgtgatcctgagaacaagaagaaggtaaagcatctattcgaattgccaaaagctgacacaaacttaacgctgtggaaagcggacttgtcagtggaaggaagctttgatgaagccattcaaggctgtcaaggagtattccatgtggcaacacctatggatttcgagtccgaggaccctgagaatgaagtaattaaaccaacagtcaggggaatgttaagcatcatagaatcatgtgctaaagcaaacacagtgaagaggctggttttcacttcatcggctggaactctcgatgcccaagaacaccaaaagcttttctatgacgagaccagctggagcgacttggacttcatatatgctaagaagatgacaggatggatgtattttgtttccaagatactggcagagaaggccgcaatggaagcagctaaaaagaagaactttgattttattagcatcataccgccgctggttgttggtccattcctcacgcctacattcccacctagcttaatcactgcactttcactaattactgggaatgaagctcactactgcatcattaaacaaggtcaatatgtgcatttggatgatctttgtgaggctcatattttcctatatgagcagccaaaggcagagggaagattcatctgcgcgtcccatcatgctatcatctatgatgtggcaaagatggtccgagagaaatggccagagtactacgtccctactgagtttaaaggcatcgataaggacttgcccgtggtgtatttttcgccaaagaagctgacggatatggggtttcaattcaagtacactttggaggatatgtataaaggggccattgagacttgtcgacagaagcagttgcttcccttttctacccaaagcacagcagataatggacgtgacaaagaaaccattcccctttctgctgaaaactatgcaagtggcaaagagaattcaccagttgcaaatggtacaggaaagtcaaccaatggggaaatctag

SEQ ID NO: 38 - DNA, 1149 bp, Nicotiana sp.
  source 1..1149: mol_type = genomic DNA
atggcaagtgaaggtcatgcagctgttcatgccccttctcctccggcagcgccgacagtttgcgtcactggagcagctggatttattggctcttggcttgtcatgagactccttgaacgtggttataatgttcacgctactgttcgtgatcctgagaacaagaagaaggtaaagcatctattggaattgccaaaagctgacacgaacttaacgttgtggaaagcagacttgtcagtggaaggaagctttgatgaagccattcaaggctgtcaaggagtattccatgtggcaacgcctatggatttcgagtccgaggatcctgagaatgaagtaattaaaccaacagtcaggggaatgttaagcatcatagaatcatgtgctaaagcaaacacagtgaagaggctggttttcacttcatctgctggaactgtcgatgtccaagagcaccaaaagctcttatatgacgagaccagctggagtgacttggactttatatatgctaagaagatgacaggatggatgtattttgtttccaagatactggcagagaaggccgcaatggaagcagctaaaaagaagaacatcgatttcattagcatcataccgccactggttgttggtccattcctcgcgcctacattcccacctagcttaatcactgccctttcactaattactgggaatgaagctcactacagcatcattaaacaaggtaaatatgtgcatttggatgatctttgtgaggctcatattttcctatatgagcacccaaaggcagaggggagattcatctgcgcgtcccatcatgctatcatctatgatgtggcaaagatggtccaagagaaatggccggagtactacgtccctactgagtttaaaggcatcgataaggacttgtccgtggtgtatttttcgtcaaagaagctgacggatatggggtttcaattcaagtacactttggaggatatgtataaaggggccattgagacttgtcgacagaagcagttgcttcccttttctacccgaagcactgcagataatgtacgtgacaaagaagccattcctctttctactgaaaactatgcaagtggcaaagaaaattcaccagttgcaaatggtacgggaaagtcaaccaatggtgaaatctag

SEQ ID NO: 39 - DNA, 642 bp, Nicotiana sp.
  source 1..642: mol_type = genomic DNA
atgggaagaaaaccctgttgttctaaagaaggtctgaggaaaggtacatggactgcaaaagaagatatgctacttactaattatattaatgaacatggagaagttggatggagatctcttcctatgaaagctggaagtcgttggtctctcattgctggaagaatacctggtcgaacagacaacgaaataaagaattattggaacacacatcttctcaagaaactcaaatccgaaggacttgagccaaaaatacacaaatctcttgcaaaaaacactagaagacaaaaggagaaagcaaatgtttcttcccaaattaaccaaaaaggttacaaggaaaagaagaagaggaataaaaagggcaatatcgaagaaaattgtaacaatattgaagagaaagaacaagtagctaagaagatagaggagcagtggcatacgcaggattccgtgcaagcgatgtcagggttttcaagtactagtgaagttgctagtgagaaggaaactaattgcaataatgtccattgtccttcttctggtcaaagtcttgaagaaaatgacaatgaaatttatgagaagcttcaggctagcggagattctaaacgttgcaaattgaatttcagtgcagaggttaataagaccccttaa

SEQ ID NO: 40 - DNA, 1308 bp, Nicotiana sp.
  source 1..1308: mol_type = genomic DNA
atgaagatcgaggtgaaagaatcgacgatggtaaagccggcggcggagacgccacaacagaggctgtggaactctaatgtggatttggtggtgccgaatttccacacgccaagtgtttatttttacaggccgacgggatccccaaatttcttcgacggaaaagtgctgaaggaagctctaagcaaagcacttgtgccgttttatcctatggcggggaggctgtgtagggacgaagatggtcgtattgagattgactgtaaaggtcagggggtgctttttgtggaagctgagtcggatggtgtggtggatgattttggtgattttgccccgacgttagaactccgtcaactcatccccgccgttgattactcacaaggaattcaatcgtatgctctcttagtgttgcagataacacattttaaatgtgggggagtttcccttggtgtgggcatgcaacatcatgcagcagatggagcttctggtcttcacttcatcaacacatggtctgatatggctcgtggtctggacctcaccatcccacctttcattgaccggaccctcctccgtgctcgtgatccacctcagcctcagtttccccatgtcgagtaccagccacctcccactctcaaggtaactccagaaaacacccctatatctgaagctgttcctgaaaccagcgtgtccatcttcaaattaacccgtgatcaaatcaataccctcaaagcgaagtccaaggaagatggaaataccgttaactacagctcctacgagatgttggcaggacatgtgtggcgctccacgtgcatggcacgaggactcgctcatgatcaagaaaccaaattgtacatagcaacagatggacgttccaggcttcggccctctctcccaccaggctatttcggtaatgtgatatttactaccactcctattgcagtcgcaggtgatatccaatcgaagcctatttggtatgctgccagtaaattacatgatgcattggctagaatggacaacgattacttaagatcagctcttgattatttggagttgcagcctgacttaaaggctcttgttcgtggtgcacatacgtttaagtgcccgaatttaggaataactagttggtctaggctgccaatccatgatgctgattttggctggggtaggcctatatttatgggacctggtggtattgcttatgaaggtttaagctttatattgccaagtcctacaaatgatggcagtcaatctgttgcaatctctctacaagcagaacacatgaaacttttcgagaagttcttgtatgacttttga

SEQ ID NO: 41 - DNA, 1311 bp, Nicotiana sp.
  source 1..1311: mol_type = genomic DNA
atgggaagtgaaaaaatgatgaaaattaatatcaaggaatcaacattagtaaaaccatcaaaaccaacaccaacaaaaagactttggagttctaacttagatttaatagtgggaagaattcatcttttaacagtatatttctataaaccaaatggatcttcaaatttctttgattcaaaaataatgaaagaagcattaagtaatgttcttgtttcattttacccaatggctggaagattagctagagatgaacaaggaagaattgagataaattgtaatggagaaggagttttatttgttgaagctgaaagtgatgcttttgttgatgattttggtgattttactccaagtttggaacttaggaaacttattcctactgttgacacttctggtgatatttctactttccccctcatcatctttcaggttactcgtttcaaatgtggtggagtttcacttggtggaggagtattccacactttatcagatggtctctcatcaattcacttcatcaacacatggtccgatatagcccgaggcctctccgtcgccatcccgccgttcatcgaccggaccctcctccgtgcacgggacccaccaacatcgtctttcgagcacgtcgagtatcatcctcctccatctctaatttcatcatcaaaaagcttagaatccactagcccaaagcctagtaccacaaccatgttaaaattctctagtgaccaacttgggcttctaaagtccaagtccaaacatgatggtagcacttacgaaatcctcgcggcccatatttggcgttgcacgtgcaaggcacgtgcactgtccgacgatcaattgaccaaattacatgtggccactgatggtaggtctaggctttgccctcctttgccaccaggttacttaggaaatgttgtgttcacaggcacacctatggcaaaatcaagtgaacttttacaagaaccattgacaaattcagccaagagaattcatagtgcattatcaaaaatggatgacaattacctaagatcagctctcgattacctcgaattactgcccgatttatcggctttaatccgtggaccgacgtactttgctagccctaatcttaatattaatagttggactagattgcctgttcatgattcagattttggatggggaaggccaattcatatgggaccagcttgcattttatatgaagggacagtttatatattgccaagtccaaatagtaaagataggaacttgcgtttggctgtttgtttagatgctgatcacatgccactatttgagaagtatttgtatgaattttga

SEQ ID NO: 42 - DNA, 2148 bp, Nicotiana sp.
  source 1..2148: mol_type = genomic DNA
atggcatcaaatggtcatgttaatggaggagaaaactttgagttgtgcaaaaaatcagctgatccattgaattgggaaatggcagctgaatccttaagagggagtcatttggatgaagtgaaaaaaatggtgagtgaatttagaaaaccaatggtaaaacttggtggtgaaagtttaacagtggcacaagtggctgctattgctgttagggacaaaagtgcaaatggtgttaaagttgaactttctgaagaggcaagagctggtgttaaagctagtagtgattgggttatggacagtatgaataaaggaactgatagttatggtgttactactggttttggtgctacatctcataggagaaccaagaatggtggtgctcttcaaaaagaacttattaggttcttgaatgctggtgtttttggcaatggaacagaaacaagccacacattgccacattcagcaacaagggcagctatgcttgttaggatcaacacactcctacaaggctactctggcatcagatttgaaatcttggaagctattacaaaattgattaacagcaacatcactccatgtttacctctccgtggaacgatcactgcctcgggtgatcttgtccctttatcctacattgctggtttgctcactggtaggcctaattccaaggctgttggtcccaatggtgagacacttaatgctgaagaagcgttccgcgttgctggtgttaacggtggatttttcgagttgcagcctaaggaaggacttgcacttgtgaatggtacagctgttggttctggtatggcatcaatggtcctctttgattccaacattcttgctgtaatgtctgaagttttatcagcaattttcgctgaagtaatgaacggaaagcccgaattcactgaccatttgacacacaagttgaagcaccaccctggtcaaattgaggctgctgctattatggaacatattttggatggaagctcttatgtgaaggcggctcaaaagctacatgaaatggatcctctacaaaaaccaaagcaagatcgttatgctctccgaacatctccacaatggcttggccctcaaattgaagtcattcgcgctgcaactaagatgattgagagggagattaactcagtgaacgataaccctttgatcgatgtttcaagaaacaaggcgttacatggtggcaacttccaaggcactcctatcggtgtttccatggataatgcaagattggctcttgcatcaattgggaaattgatgtttgctcaattctcggaacttgtcaacgactattacaacaacggtttgccctctaatctcactgcatcaaggaatccaagcttggactatggtttcaagggagctgaaatcgccatggcttcttactgctcagaacttcaattcttggcaaatccagtgacaaaccatgtccaaagtgctgaacaacacaaccaagatgtcaactccttaggcttaatctcagcaaggaaaacagctgaagctgttgatatcttaaagctcatgtcatcaacttatctcgtggcactttgccaagctatagacttgaggcatttggaagaaaacttaaagaatgcagtcaagaacacagttagccaagtagctaagagaactcttacaatgggtgctaatggtgaacttcatccagcaagattctgtgaaaaggaattgcttcgaatcgtggatagggaatacttgttcgcctacgctgatgatccttgcagttgcaactaccctttaatgcagaaactgagacaagtacttgttgatcatgcaatgaataatggtgaaagtgagaagaatgtgaacagctcaatctttcaaaagattggagctttcgaagatgaattgaaggctgttttaccaaaggaagttgagagtgcaagagctgcattagaaagtggaaaccctgctattcctaacaggattacagaatgcagatcttatccattgtacaggtttgtgagaaaggagcttggaacagaattattgacaggagaaaaagtccgatcaccgggcgaggagtgtgacaaagtgttcacagcaatgtgcaatggacaaatcattgatccaatgttggagtgtctcaagagctggaatggtgctcctcttcctatctgttag

SEQ ID NO: 43 - DNA, 1026 bp, Nicotiana sp.
  source 1..1026: mol_type = genomic DNA
atggataattcagctccagattcgttatccagatcggaaaccgccgtcacatacgactcaccatatccactctacgccatggctttctcttctctccgctcatcctccggtcacagaatcgccgtcggaagcttcctcgaagattacaacaaccgcatcgacattctctctttcgattccgattcaatgaccgttaagcctctcccgaatctctccttcgagcatccttatcctccaacaaagctaatgttcagtcctccttctctccgtcgtccttcctccggagatctcctcgcttcctccggcgatttcctccgtctttgggaaattaacgaagattcatcaaccgtcgagccaatctcggttctcaacaacagcaaaacgagcgagttttgtgcgccgttgacttccttcgattggaacgatgtagagccgaaacgtctcggaacttgtagtattgatacgacgtgtacgatttgggatattgagaagtctgttgttgagactcagcttatagctcatgataaagaggttcatgacattgcttggggagaagctagggttttcgcatcagtctctgctgatggatccgttaggatctttgatttacgtgataaggaacattctacaatcatttacgagagtcctcagcctgatacgcctttgttaagacttgcttggaacaaacaagatcttagatatatggctacgattttgatggattctaataaggttgtgattctcgatattcgttcgccgactatgcctgttgctgagcttgaaagacatcaggctagtgtgaatgctatagcttgggcgcctcagagctgtaaacatatttgttctggtggtgatgatacacaggctcttatttgggagcttcctactgttgctggacccaatgggattgatccgatgtcggtttattcggctggttcggagattaatcagttgcagtggtcttcttcgcagcctgattggattggtattgcttttgctaacaaaatgcagctccttagagtttga

SEQ ID NO: 44 - DNA, 1323 bp, Nicotiana sp.
  source 1..1323: mol_type = genomic DNA
atgagcatgtctgttgctttgttgtgggttgtttctcccacttccgaggtctcgaatgggacaggattgttggattcagtccgagaaggaaaccgcgtctttgtatcatccaggttcctagctcgagataggaatttgatgtggaatgggagaatcaagaaaggtgggagacaaaggtggaattttggctctttaattgctgatccaagatattcatgcttgggtggatcaagaactgaaaagggaagcagtttctctgtacagtccagtttggtggctagcccagctggagaaatgacagtgtcatcagagaaaaaggtctatgatgtggtattgaagcaagcagctttagtgaagaggcagctgagatctaccgatgaattagaagtgaaacctgatatagttgttccagggaatttgggcttgttgagtgaagcatatgatcgttgtggcgaagtatgtgcagagtatgcaaagacattttacttaggaacaaagctaatgactccagagagaagaagagctatctggtcaatatatgtgtggtgcaggagaacggatgagctagtcgatggccctaacgcatcacacataactccacaagctttagacaggtgggaggccaggctggaagatattttcagtgggcggccatttgatatgcttgatgctgctttatccgatactgtctccagatttcctgttgatattcagccattcagagatatgatagaaggaatgcgtatggacttgtggaaatccagatataacaacttcgatgagctatatctctattgttattatgttgctggtacagtaggactgatgagtgttccagttatgggtattgcacctgaatcaaaggcaacaacagagagtgtatataatgctgctttggctttagggcttgcaaatcaactaaccaatatactcagagatgtaggagaagatgccagaagaggacgagtatacttacctcaagatgaattagcacaggcagggctttctgatgaagatatatttgctggaagagtgaccgataagtggaggaactttatgaagaaacaaattcagagggcgaggaaattctttgatgagtcagagaaaggtgtcacagaactggactctgctagtagatggcctgtaagtacagcgctgctgttgtatcgcaagatattggacgagattgaagccaatgactacaataacttcacaaggagggcttatgttagcaagccaaagaagcttctcaccttgcccattgcttatgcaaaatctcttgtgccccctaatagaacttcctctccactagcaaaaacatga

SEQ ID NO: 45 - DNA, 1323 bp, Nicotiana sp.
  source 1..1323: mol_type = genomic DNA
atgagcatgtctgttgctttgttgtgggttgtttctcccacttccgaggtctcgaatgggacaggattgttggattcagtccgagaaggaaaccgcgtctttgtatcatccaggttcctagctcgagataggaatttgatgtggaatgggagaatcaagaaaggtgggagacaaaggtggaattttggctctttaattgctgatccaagatattcatgcttgggtggatcaagaactgaaaagggaagcagtttctctgtacagtccagtttggtggctagcccagctggagaaatgacagtgtcatcagagaaaaaggtctatgatgtggtattgaagcaagcagctttagtgaagaggcagctgagatctaccgatgaattagaagtgaaacctgatattgttgttccagggaatttgggcttgttgagtgaagcatatgatcgttgtggcgaagtatgtgcagagtatgcaaagacattttacttaggaacaaagctaatgactccagagagaagaagagctatctgggcaatatatgtgtggtgcaggagaacggatgagctagtcgatggccctaacgcatcacacataactccacaagctttagacaggtgggaggccaggctggaagatattttcagtgggcggccatttgatatgcttgatgctgctttatccgatactgtctccagatttcctgttgatattcagccattcagagatatgatagaaggaatgcgtatggacttgtggaaatccagatataacaacttcgatgagctatatctctattgttattatgttgctggtacagtaggactgatgagtgttccagttatgggtattgcacctgaatcaaaggcaacaacagagagtgtatataatgctgctttggctttagggcttgcaaatcaactaaccaatatactcagagatgtaggagaagatgccagaagaggacgagtatacttacctcaagatgaattagcacaggcagggctttctgatgaagatatatttgctggaagagtgaccgataagtggaggaactttatgaagaaacaaattcagagggcgaggaaattctttgatgagtcagagaaaggtgtcacagaactggactctgctagtagatggcctgtgttagcagcgctgctgttgtatcgcaagatattggacgagattgaagccaatgactacaataacttcacaaggagggcttatgttagcaagccaaagaagcttctcaccttgcccattgcttatgcaaaatctcttgtgccccctaatagaacttcctctccactagcaaaaacatga

SEQ ID NO: 46 - DNA, 747 bp, Nicotiana sp.
  source 1..747: mol_type = genomic DNA
atggagggttcgtccaaagggctgcgaaaaggtgcttggactactgaagaagatagtctcttgagacagtgcattaataagtatggagaaggcaaatggcaccaagttcctgtaagagctgggctaaaccggtgcaggaaaagttgtagattaagatggttgaactatttgaagccaagtatcaagagaggaaaacttagctctgatgaagtcgatcttcttcttcgccttcataggcttctagggaataggtggtctttaattgctggaagattacctggtcggaccgcaaatgacgtcaagaattactggaacactcatctgagtaagaaacatgaaccgtgttgtaagataaagatgaaaaagagagacattacgcccattcctacaacaccggcactaaaaaacaatgtttataagcctcgacctcgatccttcacagttaacaacgactgcaaccatctcaatgccccaccaaaagttgacgttaatcctccatgccttggacttaacatcaataatgtttgtgacaatagtatcatatacaacaaagataagaagaaagaccaactagtgaataatttgattgatggagataatatgtggttagagaaattcctagaggaaagccaagaggtagatattttggttcctgaagcgacgacaacagaaaagggggacaccttggcttttgacgttgatcaactttggagtcttttcgatggagagactgtgaaatttgattag

SEQ ID NO: 47 - protein, 717 aa, Nicotiana sp.
MASNGHVNGGENFELCKKSSATDPLNWEMAAESLRGSHLDEVKKMVSEFRKPMVKLGGETLTVAQVAAIAVRDKSANGVKVELSEEARAGVKASSDWVMDSMNKGTDSYGVTTGFGATSHRRTKNGGALQKELIRFLNAGVFGNGTETSHTLPHSATRAAMLVRINTLLQGYSGIRFEILEAIAKLINSNITPCLPLRGTITASGDLVPLSYIAGLLTGRPNSKAVSPNGETLNAEEAFRVAGVNGGFFELQPKEGLALVNGTAVGSGMASMVLFDSNILAVMSEVLSAIFAEVMNGKPEFTDHLTHKLKHHPGQIEAAAIMEHILDGSSYVKAAQKLHEMDPLQKPKQDRYALRTSPQWLGPQIEVIRAATKMIEREINSVNDNPLIDVSRNKALHGGNFQGTPIGVSMDNARLALASIGKLMFAQFSELVNDYYNNGLPSNLTASRNPSLDYGFKGAEIAMASYCSELQFLANPVTNHVQSAEQHNQDVNSLGLISARKTAEAVDILKLMSSTYLVALCQAIDLRHLEENLKNAVKNTVSQVAKRTLTMGANGELHPARFCEKELLRVVDREYLFAYADDPCSCNYPLMQKLRQVLVDHAMNNGESEKNVNSSIFQKIGAFEDELKAVLPKEVESARAALECGNPAIANRITECRSYPLYRFVRKELGTELLTGERVRSPGEECEKVFTAMCNGQIIDPMLECLKSWNGAPLPIC

SEQ ID NO: 48 - protein, 712 aa, Nicotiana sp.
MAGVAQNGHQEMDFCMKVDPLNWEMAADSLKGSHLDEVKKMVAEFRKPVVKLGGETLTVAQVAAIAAKDNVKTVKVELSEGARAGVKASSDWVMDSMGKGTDSYGVTTGFGATSHRRTKNGGALQKELIRFLNAGVFGNGTESCHTLPQSGTRAAMLVRINTLLQGYSGIRFEILEAITKLLNHNVTPCLPLRGTITASGDLVPLSYIAGLLTGRPNSKAVGPNGETLNAEEAFRVAGVNGGFFELQPKEGLALVNGTAVGSGLASMVLFDANVLAVFSEVLSAIFAEVMNGKPEFTDHLTHKLKHHPGQIEAAAIMEHILDGSSYVKAAQKLHETDPLQKPKQDRYALRTSPQWLGPQIEVIRSATKMIEREINSVNDNPLIDVSRNKALHGGNFQGTPIGVSMDNARLALASIGKLMFAQFSELVNDYYNNGLPSNLTAGRNPSLDYGFKGSEIAMASYCSELQFLANPVTNHVQSAEQHNQDVNSLGLISARKTAEAVDILKLMSSTYLVALCQAIDLRHLEENLRNAVKNTVSQVAKRTLTMGTNGELHPSRFCEKDLLRVVDREYVFAYADDACSANYPLMQKLRQVLVDHALQNGENEKNANSSIFQKILAFEDELKAVLPKEVESARAALESGNPAIANRIKECRSYPLYRFVRGELGAELLTGEKVRSPGEECDKVFTAMCNGQIIDSLLECLKEWNGAPLPIC

SEQ ID NO: 49 - protein, 389 aa, Nicotiana sp.
MVTVEEFRRAQRAEGPATVMAIGTATPSNCVDQSTYPDYYFRITNSEHKTELKEKFKRMCEKSMIKKRYMHLTEEILKENPNICAYMAPSLDARQDIVVVEVPKLGKEAAQKAIKEWGQPKSKISHLVFCTTSGVDMPGCDYQLTKLLGLRPSVKRFMMYQQGCFAGGTVLRMAKDLAENNKGARVLVVCSEITAVTFRGPNDTHLDSLVGQALFGDGAAAVIVGSDPIPDVERPLFELVSAAQTLLPDSEGAIDGHLREVGLTFHLLKDVPGLISKNIEKSLVEAFQPLGISDWNSLFWIAHPGGPAILDQVELKLGLKQEKLKATRNVLSNYGNMSSACVLFILDEMRKASAKEGLGTTGEGLEWGVLFGFGPGLTVETVVLHSVAT

SEQ ID NO: 50 - protein, 369 aa, Nicotiana sp.
MAPSTLTALAEEKTLQTSFIRDEDERPKVAYNQFSDEIPIISLKGIDDEGGINGRRGEICEKIVKACEDWGVFQVVDHGVDAQLISQMTTLAKQFFALPAEEKLRFDMSGGKKGGFIVSSHLQGEVVQDWREIVTYFSYPIRARDYSRWPDKPEGWIDVTQKYSEKLMELACKLLEVLSEAMGLEKEALTKACVDMDQKVVVNFYPKCPQPDLTLGLKRHTDPGTITLLLQDQVGGLQATKDNGKTWITVQPVVGAFVVNLGDHGHFLSNGRFKNADHQAVVNSNSSRLSIATFQNPAPEAIVYPLKIREGEKAVMDEPVAFAEMYRRKMSKDLELARLKKLAKEQQIQAEEAAEKAKSETKPIDEILA

SEQ ID NO: 51 - protein, 145 aa, Nicotiana sp.
MFGPQSGKDGWTDLTFMYDMIRIRDKSLCSSFLQIFSSEGCKMLEMTCEEHDKLAARSQFLTHTIGRILSEMEVEPTPIDTKGFQKLVQVKESSVRDSFDLFSGLFIHNRFARQQMKNLEVAVEKTKQKLEERSKELQDPIISKF

SEQ ID NO: 52 - protein, 376 aa, Nicotiana sp.
MLSFTPLQSKPTPPTPTSNPTRWSWSHLTHHHPTTRRHFSASSPSKVSYRRLSINAIDAAQPYDYEALVSNQYAQSTRLKIAIVGFGNFGQFLAKAFVRQGHVVFAHSRTDYSHIANSLGVLFFQDPHDLCEQHPDVILLCTSIISTEPVLRSLPIQRLKRNTLFVDVLSVKEFPKNIFLQVLPSHFDILCTHPMFGPESGKDSWKDLAFVFDKVRIGEGESRKGRVDRFLDIFEKEGCRMVQMTCAEHDRYAAGSQFITHTMGRVLEKLDLETTPINTKGYETLLNLVENTSSDSFDLYYGLFMYNKNAMEQLERLDLAFEALKKELFGHLHEVLRKQLFGKAEEAGQRRILTKLPKNGYALPAPSSEAVKSENN

SEQ ID NO: 53 - DNA, 2154 bp, Nicotiana sp.
  source 1..2154: mol_type = genomic DNA
atggcatcaaatggtcatgttaatggaggagaaaactttgagttgtgcaagaaatcatcagccactgatccattgaattgggaaatggcagctgaatctttaagagggagtcatttggatgaagtgaaaaaaatggtgagtgaatttagaaaaccaatggtaaaacttggtggtgaaactttaacagtggcacaagtggctgctattgctgttagggacaaaagtgcaaatggtgttaaagttgaactttctgaagaggcaagagctggtgttaaagctagtagtgattgggttatggatagtatgaataaaggaacagatagttatggtgttactactggttttggtgctacatctcataggagaaccaagaatggtggtgctcttcaaaaagaacttattaggttcttgaatgctggtgtttttggcaatggaacagaaacaagccacacattgccacattcagcaacaagggcagctatgcttgttaggatcaacacactcctacaaggctactctggcatcagatttgaaatcttggaagcaattgcaaaattgattaacagcaacattactccatgtttacctctccgtggcacgatcactgcctcgggcgatcttgttcctttatcctacattgctggtttgctcactggtaggcctaattccaaggctgttagtcccaatggtgagacccttaatgctgaagaagcgttccgcgttgctggtgttaacggtggatttttcgagttgcagcctaaggaaggacttgcacttgtgaatggtacagcagttggttctggtatggcatcaatggtcctctttgattccaacattcttgctgttatgtctgaagttttatcagcaattttcgctgaagttatgaacggaaagcccgaatttactgaccatttgacacacaagttgaagcaccaccctggtcaaattgaggctgctgctattatggaacatattttggatggaagctcttatgtgaaggcggctcaaaagctacatgaaatggatcctctccaaaaaccaaagcaagatcgttatgctctccgaacatctccacaatggcttggccctcaaattgaagtcattcgcgctgcaactaagatgattgagagggagattaactcagtgaacgataaccccttgatcgatgtttcaagaaacaaggcattacatggtggcaacttccaaggcacccctatcggtgtgtccatggataatgcaagattggctcttgcatcaattgggaaattgatgtttgctcaattctcggaacttgtcaacgactattacaacaacggtttgccatctaacctcaccgcatcaaggaatccaagcttggactatggtttcaagggagctgaaatcgccatggcatcttactgctcagaacttcaattcttggcaaatccagtgacaaaccatgtccaaagtgctgagcaacacaaccaagatgtcaactccttgggcttaatctcagcaaggaaaacagctgaagctgtcgatatcttaaagctcatgtcatcaacttatctagtggcactttgccaagctatcgacttgaggcatttggaggaaaacttaaagaatgcagtcaagaacacagttagccaagtagctaagagaactcttacaatgggtgctaacggtgaacttcatccagcaagattctgtgaaaaggaattgctacgagtcgtggacagggaatacttgttcgcctacgctgatgatccttgcagttgcaactaccctttaatgcagaaactgagacaagtacttgttgatcatgcaatgaataatggtgaaagtgagaagaatgtgaacagctcaatcttccaaaagattggagctttcgaagacgaattaaaggctgttttaccaaaggaagttgagagtgcaagagctgcattggaatgtggcaaccctgctattgctaacaggattacagaatgcagatcttatccattgtacaggtttgtgagaaaggagcttggaacagaactactaacaggagaaagagtccgatcaccgggcgaggagtgtgagaaagtgttcacagcaatgtgcaatggacagattattgatccaatgttggagtgtctcaagagctggaatggtgctcctctacctatctgttag

SEQ ID NO: 54 - DNA, 2139 bp, Nicotiana sp.
  source 1..2139: mol_type = genomic DNA
atggctggtgttgcacaaaatggtcaccaagaaatggatttttgcatgaaagtggatccattaaactgggaaatggcagctgattcattgaaaggaagccatttagatgaagtgaagaaaatggtggctgagtttaggaaaccagtagtgaaacttggaggtgagactttgacagtggctcaagttgcggctattgctgcaaaagataatgttaaaactgttaaagtggagctttctgaaggggcaagagctggtgttaaagctagcagtgattgggttatggacagtatgggtaaaggaactgatagttatggtgttacaactggctttggtgctacttcacataggaggaccaagaatggtggtgctcttcaaaaggaacttattaggttcttgaatgctggagtttttggcaatggaacagagtcatgtcacacattaccacaatcagggacaagggcagctatgttagttaggatcaacactctccttcaagggtactctggcatcagatttgaaatcttagaagcaatcactaaattgcttaaccacaatgttactccatgtttgccccttcgcggcaccatcaccgcctctggtgatctcgtccccttgtcctacattgccggtttactcactggtcggcctaattctaaagcagttggacctaatggcgaaaccctcaacgctgaagaagcgtttcgtgttgctggagttaacggtggatttttcgagttgcagcctaaggaaggccttgctcttgtgaatggtactgcagttggttctggtttggcctcaatggttctctttgatgctaatgttctcgcggtcttttctgaagttctctcagctatttttgctgaggtaatgaatggaaagcccgagttcactgaccacttgacacacaagttgaagcatcaccccggacaaattgaggctgctgctattatggaacacattttggatggtagctcttatgtgaaggcggctcagaagcttcacgaaacggatcctctccaaaaaccaaagcaagatcgttatgctcttagaacgtcgccccaatggcttggccctcaaattgaggtcatccgttctgcaaccaagatgattgagagggagattaattcagtgaacgacaaccctttgatcgatgtttcaagaaacaaggcattacacggtggcaacttccagggcactccaattggtgtctctatggacaatgctagattagcccttgcatcaatagggaaattgatgtttgcccaattctccgagcttgtcaacgattactacaacaacggattgccatctaatctgacagcaggaaggaatcctagcttggactatggtttcaagggatctgagattgccatggcttcatactgttcagaacttcaattcttggcaaatccagtgactaaccacgtacaaagcgccgagcaacacaaccaagatgtgaactccttgggcttaatctcagctagaaaaacagctgaagccgtggacatcttaaagctaatgtcatccacatatctagttgcactttgccaagcaatagacttgaggcatttggaagaaaatctgaggaatgcagtcaagaacacggtgagccaagtcgcaaagagaactttaacaatgggtaccaatggagaacttcatccatcaagattctgtgaaaaggacttgcttcgagtcgtggacagggaatacgtcttcgcctatgctgacgacgcctgcagcgctaactacccactgatgcagaaactaaggcaagtcctcgtcgaccacgccttgcaaaatggcgaaaatgagaagaacgcaaacagctcaatcttccaaaagatactagcttttgaagacgagctaaaggccgtgttgccaaaagaagtcgagagtgcaagagccgcgctggaaagtgggaaccctgcaattgccaacaggataaaagaatgcagatcttatccactttacaggtttgttagaggagaacttggagctgaattattgacgggagaaaaagtcaggtcaccaggtgaagaatgtgacaaagtgttcacagcaatgtgcaatggacaaattattgattcattgttagaatgtctcaaggaatggaatggtgcacctcttccaatctgttag

SEQ ID NO: 55 - DNA, 1170 bp, Nicotiana sp.
  source 1..1170: mol_type = genomic DNA
atggtgacggtcgaggagtttcgtagggcacaacgtgccgagggtccggccacggtcatggccatcggaacagccacaccttccaactgtgttgatcaaagcacttaccccgattactattttcgtatcactaatagcgagcataagactgagcttaaggagaaatttaagcgcatgtgtgaaaaatcaatgattaagaaaaggtacatgcacttaacagaggaaatcttgaaagagaatcctaatatttgtgcatacatggcaccttcccttgatgctagacaagacatagtggtggttgaagtgccaaaacttggcaaagaggcagcccaaaaggccatcaaagaatggggccagcccaagtccaaaattagtcatttggtcttttgtacaactagtggtgtagacatgcccgggtgtgactaccaactcactaagctactcgggctccgtccttcggtcaagcggttcatgatgtaccaacaaggttgctttgccggtgggacggtactccggatggctaaggacttggccgaaaacaacaagggcgctcgagtccttgttgtttgctcagagatcaccgctgtcacgttccgtgggcccaatgacacccacttggatagtttggttgggcaagccctttttggtgatggggcagccgcggtcattgtaggttctgatccaattccagatgtcgagaggcctttgttcgagcttgtttccgcagcccaaaccctactccccgatagcgaaggcgctatcgacggtcatctccgtgaagttgggcttacattccacttactcaaagatgttcctgggcttatctcgaaaaacattgagaaaagccttgtggaagcattccaacctttgggaatttctgattggaactctttattttggattgctcaccctggtgggcctgccattttggaccaagttgaactaaaattgggcctaaagcaagagaaacttaaggctacaagaaatgtattaagtaactatggcaatatgtcaagtgcttgtgtgttgtttattttggatgaaatgaggaaagcctctgcaaaagaaggtttaggaactactggtgaagggcttgaatggggtgtgctttttggatttgggcctgggcttacagttgagactgttgtccttcacagtgttgctacttag

SEQ ID NO: 56 - DNA, 1110 bp, Nicotiana sp.
  source 1..1110: mol_type = genomic DNA
atggcaccttcgacattgacagctctagcagaggaaaagacacttcaaacaagtttcataagagatgaagatgagcgaccaaaagtggcgtacaaccaattcagcgatgagattccgatcatatcgttgaagggtattgatgatgagggtggaattaatggaagaagaggtgaaatatgtgaaaagattgtcaaggcatgtgaagattggggcgttttccaggtagttgatcatggtgttgatgctcaacttatctcacaaatgacaactctcgctaaacaattcttcgctttgcctgctgaggaaaagctacggtttgacatgtcgggtggcaagaaaggtggcttcattgtctctagccatctacagggtgaagtggtccaagattggcgtgaaatagtgacctacttctcatacccaattcgggctagagactactctagatggccagacaaaccagagggatggatagatgtgactcaaaagtacagtgaaaagttaatggagttggcttgcaaattattggaagtactatcagaggctatgggcttagagaaggaggccttaaccaaggcatgtgtggatatggaccaaaaagtggttgtcaatttttacccaaagtgtccacagcctgaccttacccttgggctgaaacgacacactgatccaggaaccatcaccctcttgttacaagaccaagttggtgggcttcaagccactaaagataatggcaaaacttggattactgttcagcccgttgttggcgcttttgttgtcaatcttggtgaccatggtcattttttgagcaatggaaggtttaagaacgctgatcatcaagcagtggtgaactcgaatagcagcagattatcgatagctacgtttcagaatccagcaccagaggcaatagtgtatcctttgaaaattagggaaggagagaaggcagtgatggacgagcccgtagcatttgcagaaatgtataggaggaaaatgagtaaggaccttgagcttgctaggctcaagaaactagccaaggagcagcaaatacaagctgaagaagctgctgagaaggccaagtcggaaaccaagcctattgatgaaattcttgcttaa

SEQ ID NO: 57 - DNA, 438 bp, Nicotiana sp.
  source 1..438: mol_type = genomic DNA
atgtttggaccacaaagtggaaaagatggatggactgatttgacttttatgtacgacatgattcgaattagagataaatctctgtgttccagttttctgcaaatattctcaagtgaggggtgcaaaatgctggaaatgacttgtgaagagcatgacaaattggctgctcgaagtcaatttctgactcacacaattggcaggatcttatccgaaatggaggttgaacccacccccatagacacgaagggatttcagaaacttgttcaagtgaaggagagctcagttagagatagttttgatctattcagcgggctattcatacacaataggtttgccaggcaacagatgaaaaatttagaagtagcagtggagaaaactaaacagaagcttgaagagaggtcgaaggagctgcaggatcctatcatatctaagttctag

SEQ ID NO: 58 - DNA, 1131 bp, Nicotiana sp.
  source 1..1131: mol_type = genomic DNA
atgttgtctttcacccctcttcaatccaagccaacaccacccacccccacctcaaaccccacccgttggtcttggtctcatctcacccaccaccaccccactacccgccgccacttctctgcctcttcaccctccaaagtcagttaccgccgccttagcattaatgccattgatgcagcacagccatatgattatgaagcattagtttcaaatcaatatgctcagtcaacaagactcaagattgctatagtgggttttggcaacttcggtcagtttcttgctaaagcctttgttcgtcaaggtcatgttgtttttgctcattcaagaactgattactcacacattgcaaattctttaggtgttttgttttttcaagatccacatgacctttgtgagcaacatcctgatgttattctactttgtacttcaattatatctactgaacctgtccttagatcactccctattcaaaggctaaaaagaaacacattatttgttgatgttttgtctgttaaagagtttccaaagaacattttccttcaagttttgccttcccattttgatattttgtgtactcatcctatgtttggacctgaaagtggtaaggatagttggaaagatttggcctttgtgtttgataaagttagaattggtgaaggggaatcgagaaaaggaagggttgataggtttcttgatatatttgagaaagaagggtgtaggatggtgcagatgacgtgtgcggagcatgataggtatgctgcaggttcgcagtttattacacatacaatggggagagtattggagaagttggatttggagacaactcctattaatacgaaagggtacgagactttgttgaatttggttgagaatacttctagtgatagctttgacttgtactatgggttgtttatgtacaataagaatgcgatggagcagttggaaagacttgatttggctttcgaggctttgaagaaggagttatttgggcatttgcatgaagtcttgaggaagcaattgttcgggaaggcggaggaagcgggacagagacgtatcttaaccaagttgcccaagaatgggtatgcactaccagctccttcatcggaggctgttaaatctgagaacaattga

SEQ ID NO: 59 - DNA, 2623 bp, synthetic construct
  misc_feature 1..2623: note = Chimera: comprises plant native sequences only
  source 1..2623: mol_type = other DNA
cccgggttagtattcaaaccgaataaatcaaagttaccaaaccgaataaatcgaaaccgaaaggagaaaccgcaccataccgaatttaattaggtacgatattgatattgtattttaaaaaatcgaataccaaaaataccgaattaaaatatctaaatatcgtacagtaccgaccgataaatactctataagcatcatcatgtcaccattcctgcttggaaatagatgtaataatgtaattcaaggtgaagatcattgaaaatgagatatttggactcttagataattgtgcaactgatatttttatttactttttttctttcatccaataattgcgttacattaaaaatgagatatttggattaatattcttctccttgaccacaaagcaaggaaagcctaaggaccgatagtaaagttgtattcgtgtggttgcgtgttagttttgagcggcaaaataaattatgttaaggtaaattatttttggaacaataataaaattatttctgtataatatataaatcatatatttgaaccgtagaattatcagttaatacttgtatatgaggaggctaactacgttagagcgctaacgagaatacttcatataccgtattttttacgataataataatgtaatgtgaaattgctatccaaaaggcacctaattttgtccaccgttcaaaggaaaggacaaggaagtagtagcgtgtaggtttggtgctgtacaaaataagcaagacacgtgttgccttattataggataatccataaggcaatttcgtcttaagtcggccattgcacctttaaaaggagcctctttgttcccaaaatcttcatcctttgatttctctattctcaatatctcctcaatttttctctagtcttcaaacacttctcaaggtacattaacttcttctttctttttgttcctcttattttatgctacttttatttaatttcgatctatatttttaggatctaaatactcatttttgatttgtttaatcgctctgtatatatgcaccaagttgaaatttttgtaagtttattttgttcggtctatattttaagatctgaaataccctttactgagaaaaaaaaaactcaaccttgattttgttgtacctggttgaatttgttattgttgtgtatacagttaaaaaactcaagtcttgattttattgtttcccttttgtagtttgtatatacatagagctgaattggtgttctaattttggttgatttttatgtatacagtataaaatcgatcttagttttgttcattgatttgtatttgcacaaagttggaattttgcgtttgttattttgatgattgaaaccttttctgtatatacagctcgagatggagggttcgtccaaagggctgcgaaaaggtgcttggactactgaagaagatagtctcttgagacagtgcattaataagtatggagaaggcaaatggcaccaagttcctgtaagagctgggctaaaccggtgcaggaaaagttgtagattaagatggttgaactatttgaagccaagtatcaagagaggaaaacttagctctgatgaagtcgatcttcttcttcgccttcataggcttctagggaataggtggtctttaattgctggaagattacctggtcggaccgcaaatgacgtcaagaattactggaacactcatctgagtaagaaacatgaaccgtgttgtaagataaagatgaaaaagagagacattacgcccattcctacaacaccggcactaaaaaacaatgtttataagcctcgacctcgatccttcacagttaacaacgactgcaaccatctcaatgccccaccaaaagttgacgttaatcctccatgccttggacttaacatcaataatgtttgtgacaatagtatcatatacaacaaagataagaagaaagaccaactagtgaataatttgattgatggagataatatgtggttagagaaattcctagaggaaagccaagaggtagatattttggttcctgaagcgacgacaacagaaaagggggacaccttggcttttgacgttgatcaactttggagtcttttcgatggagagactgtgaaatttgattagtctagaaataacagagggcgcgcgagcggtggctactgatcgcctatgagttctgtgattctacttgtaatttcagaagtgttttcagtgtcttgttttctggaagtccgtctggtttttagtaacttttagctcaaaaatgtgtctgtacgatggtatttgtatgtttgtgggtcttttacatatacgcttgtaatcgatcaatgtagaatgctgtgtgccttttccgtcaacagcttatttagtgtttactctgtatacgtatatctaatatatagtactgattctttcatctggtgatttgttttcctaaagagattattatcatagctttaattgaatgatacaaagaggtgttgcctggcttcaccagagcagaaattttcattgatatagggtacaaatgtcattcacataatgttaagagataagtttttcaatgtcctcaagagcccaccaagagtttcttccgggaattgcttaaattatcttaaatttaaattgtaaaac

SEQ ID NO: 60 - DNA, 2539 bp, synthetic construct
  misc_feature 1..2539: note = Chimera: comprises plant native sequences only
  source 1..2539: mol_type = other DNA
cccgggttagtattcaaaccgaataaatcaaagttaccaaaccgaataaatcgaaaccgaaaggagaaaccgcaccataccgaatttaattaggtacgatattgatattgtattttaaaaaatcgaataccaaaaataccgaattaaaatatctaaatatcgtacagtaccgaccgataaatactctataagcatcatcatgtcaccattcctgcttggaaatagatgtaataatgtaattcaaggtgaagatcattgaaaatgagatatttggactcttagataattgtgcaactgatatttttatttactttttttctttcatccaataattgcgttacattaaaaatgagatatttggattaatattcttctccttgaccacaaagcaaggaaagcctaaggaccgatagtaaagttgtattcgtgtggttgcgtgttagttttgagcggcaaaataaattatgttaaggtaaattatttttggaacaataataaaattatttctgtataatatataaatcatatatttgaaccgtagaattatcagttaatacttgtatatgaggaggctaactacgttagagcgctaacgagaatacttcatataccgtattttttacgataataataatgtaatgtgaaattgctatccaaaaggcacctaattttgtccaccgttcaaaggaaaggacaaggaagtagtagcgtgtaggtttggtgctgtacaaaataagcaagacacgtgttgccttattataggataatccataaggcaatttcgtcttaagtcggccattgcacctttaaaaggagcctctttgttcccaaaatcttcatcctttgatttctctattctcaatatctcctcaatttttctctagtcttcaaacacttctcaaggtacattaacttcttctttctttttgttcctcttattttatgctacttttatttaatttcgatctatatttttaggatctaaatactcatttttgatttgtttaatcgctctgtatatatgcaccaagttgaaatttttgtaagtttattttgttcggtctatattttaagatctgaaataccctttactgagaaaaaaaaaactcaaccttgattttgttgtacctggttgaatttgttattgttgtgtatacagttaaaaaactcaagtcttgattttattgtttcccttttgtagtttgtatatacatagagctgaattggtgttctaattttggttgatttttatgtatacagtataaaatcgatcttagttttgttcattgatttgtatttgcacaaagttggaattttgcgtttgttattttgatgattgaaaccttttctgtatatacagctcgagatgaatatttgtactaataagtcgtcgtcaggagtgaagaaaggtgcatggactgaagaagaagatgttctattgaaaaaatgcatcgagaaatatggagaaggaaagtggcatcaagttcctcttagagctggtttgaatagatgcagaaagagctgcagattaaggtggctaaattatctaaggccacatataaagagaggagacttctcttttgatgaagtagatctcattttgaggcttcataagctgttaggcaacagatggtcacttattgctggtagacttcctggaaggacggcaaacgatgtcaaaaactactggaacagccatcttcgcaagaagttaattgctcctcatgatcaaaaggagagcaagcaaaaagcaaagaagatcaccatattcagacctcggcctcgaaccttctcaaagacaaatacttgtgttaaaagtaacacaaatactgtagataaggatattgaaggcagcagcgaaataattagattcaacgataatttgaagccaacaactgaagaattgacggatgatggaattcaatggtgggccgatttactagctaacaattacaacaataatgggattgaggaagctgataattcatcaccaactttgttgcatgaggaaatgccacttctcagttgatctagaaataacagagggcgcgcgagcggtggctactgatcgcctatgagttctgtgattctacttgtaatttcagaagtgttttcagtgtcttgttttctggaagtccgtctggtttttagtaacttttagctcaaaaatgtgtctgtacgatggtatttgtatgtttgtgggtcttttacatatacgcttgtaatcgatcaatgtagaatgctgtgtgccttttccgtcaacagcttatttagtgtttactctgtatacgtatatctaatatatagtactgattctttcatctggtgatttgttttcctaaagagattattatcatagctttaattgaatgatacaaagaggtgttgcctggcttcaccagagcagaaattttcattgatatagggtacaaatgtcattcacataatgttaagagataagtttttcaatgtcctcaagagcccaccaagagtttcttccgggaattgcttaaattatcttaaatttaaattgtaaaac

SEQ ID NO: 61 - DNA, 3701 bp, synthetic construct
  misc_feature 1..3701: note = Chimera: comprises plant native sequences only
  source 1..3701: mol_type = other DNA
cccgggaaactaataagaacaagcaatataagagaattcgtaaaaaactttctagttcttgaacatctacccatgtgaattctcttttatttttgtacactataattaaattaacatggctaaaccaattatgccaactgaataaattatcaatattgataaacttcaggtttacaccatgacgtgtgcaattatgaataaactccaagtttattatgatatgagtttttatatcaataaacttctactttactgtgacattcttttgtttgtgtagtacaaattggagaataaagcgtgtaacttttcacatacatattaccccgctataagttgtagtaatagaagatattaaatctttcctttctttatagtctcaatataaccaatcgcttttgcgaatactttagaaactaaataagtttctttgagacttactacaacataatgccttattgataatcaattttgttccttcaaaatagtaataattggctcttccagagctctcaattaattttgtactaccacatattcatcaacatccatatggtgaatatctcttttaaagagaaagttataccattatggttatggtcaaaattatatgcaatatttgtttcttgcattaccgttgccttttagtaatcacattgccaaaatattttggcctacaataagtacgtgaccaatgaccatttatgccataatgacattattttcttattttctctcaaacctccttcaagagatgagtgtggtatatactactagcacgctcatattcttccaaaaatgaatatgtattcataaatcacatattgtcacattcacatcatgaatggaccataatcatctatatgtgctttacaaaatctcatgtcaaatcgatgataaatattactttcacatagtgaatgattattttgagaatacttattattctcattaccacaatgaaaatgattataatttcgtctattgtcacatccacatccattgatacattcatggtaataattttatcttctttcagacgtatcatatattgctatcacattctcgtaagggaatgagaataaagcaaattcaatgggacgagtttccactttttgtgctcacaatcatacatgaatttgatcatggcaatacatagaaattttatcacttttggtggttataatttcttacaaactctattagagttataataaaaatttattgtctttgcttgtatttaaaatcaaattataaaattttaaaaattcaaaagagaaaaataaagtaaaatacttactttaaatccagaatttaatcacgaaggaagttcatggaataattgacgatcattatgctcaatcccaaagcttctactcaattggttacagtctcgtgccgataacgtgttataaaacaataaaagaagaacaatattgcagagaaagagagagagggaattcttattgaattttaggatgaattacaatggaataggacccctctatttatagggaaagagtgacttagccaccaagtaaaatccctaaaatctctctaaaatatagacattcaccttaaataaaactctatttataacaaaaacaaaattgttctagacgagaatcattttcactttttaatgtttttcttctactcctttagtttttccttaaaccaaaataaaaaaaaagtctatatttttctatcctttttcttctctagttttaatacattccataaaatttgtggtgaaattgtactttctccttttttttcctctttagtttcatagaaattaaacaataaaacgaaaacatcaaaatatatcaacaaatagtaaatttcattacatttatttttttgctcaaagtgctcttttgttgaatactcaaagtatatagtatattacagttctaatcaggaattattcactgaaatattgactgattttaggttcgtgtgatcgattaaatacctcttgaaccactacaaatttaacgaattaataacttttacctaacattttgttacccatgtattaatttgaatttatactattacttcctatttatgagtaatataacataatttctgtgtaaaggacagaaaatacagaatttgattaattcaaaattaattatacttcaaaaaatatcacaaggatatcaaaatttatcaaataaaggagcaacgtcattttcggctctcccacacaattcaaaataattcaaaacagaaccggctagacctaacctaaccaaaccatggttaatacaaatctaaatcaccgaaaaacaatcttaaccatagttaataacccgtattaattaacccgatatccatttcgccttgcccttattcattctcctataaaacagaggcccttcagtatagcgaagactcacaattcgaatttcgaaaactcatctctttctgttttaacggcgtcttgataaacgccgctccttctctcctcctctttgaaatttgaatttttagggtttcgtgaaactcgagaacaatgaatatttgtactaataagtcgtcgtcaggagtgaagaaaggtgcatggactgaagaagaagatgttctattgaaaaaatgcatcgagaaatatggagaaggaaagtggcatcaagttcctcttagagctggtttgaatagatgcagaaagagctgcagattaaggtggctaaattatctaaggccacatataaagagaggagacttctcttttgatgaagtagatctcattttgaggcttcataagctgttaggcaacagatggtcacttattgctggtagacttcctggaaggacggcaaacgatgtcaaaaactactggaacagccatcttcgcaagaagttaattgctcctcatgatcaaaaggagagcaagcaaaaagcaaagaagatcaccatattcagacctcggcctcgaaccttctcaaagacaaatacttgtgttaaaagtaacacaaatactgtagataaggatattgaaggcagcagcgaaataattagattcaacgataatttgaagccaacaactgaagaattgacggatgatggaattcaatggtgggccgatttactagctaacaattacaacaataatgggattgaggaagctgataattcatcaccaactttgttgcatgaggaaatgccacttctcagttgatctagaaataacagagggcgcgcgagcggtggctactgatcgcctatgagttctgtgattctacttgtaatttcagaagtgttttcagtgtcttgttttctggaagtccgtctggtttttagtaacttttagctcaaaaatgtgtctgtacgatggtatttgtatgtttgtgggtcttttacatatacgcttgtaatcgatcaatgtagaatgctgtgtgccttttccgtcaacagcttatttagtgtttactctgtatacgtatatctaatatatagtactgattctttcatctggtgatttgttttcctaaagagattattatcatagctttaattgaatgatacaaagaggtgttgcctggcttcaccagagcagaaattttcattgatatagggtacaaatgtcattcacataatgttaagagataagtttttcaatgtcctcaagagcccaccaagagtttcttccgggaattgcttaaattatcttaaatttaaattgtacccgtttaaac

SEQ ID NO: 62 - DNA, 6230 bp, synthetic construct
  misc_feature 1..6230: note = Chimera: comprises plant native sequences only
  source 1..6230: mol_type = other DNA
cccgggttagtattcaaaccgaataaatcaaagttaccaaaccgaataaatcgaaaccgaaaggagaaaccgcaccataccgaatttaattaggtacgatattgatattgtattttaaaaaatcgaataccaaaaataccgaattaaaatatctaaatatcgtacagtaccgaccgataaatactctataagcatcatcatgtcaccattcctgcttggaaatagatgtaataatgtaattcaaggtgaagatcattgaaaatgagatatttggactcttagataattgtgcaactgatatttttatttactttttttctttcatccaataattgcgttacattaaaaatgagatatttggattaatattcttctccttgaccacaaagcaaggaaagcctaaggaccgatagtaaagttgtattcgtgtggttgcgtgttagttttgagcggcaaaataaattatgttaaggtaaattatttttggaacaataataaaattatttctgtataatatataaatcatatatttgaaccgtagaattatcagttaatacttgtatatgaggaggctaactacgttagagcgctaacgagaatacttcatataccgtattttttacgataataataatgtaatgtgaaattgctatccaaaaggcacctaattttgtccaccgttcaaaggaaaggacaaggaagtagtagcgtgtaggtttggtgctgtacaaaataagcaagacacgtgttgccttattataggataatccataaggcaatttcgtcttaagtcggccattgcacctttaaaaggagcctctttgttcccaaaatcttcatcctttgatttctctattctcaatatctcctcaatttttctctagtcttcaaacacttctcaaggtacattaacttcttctttctttttgttcctcttattttatgctacttttatttaatttcgatctatatttttaggatctaaatactcatttttgatttgtttaatcgctctgtatatatgcaccaagttgaaatttttgtaagtttattttgttcggtctatattttaagatctgaaataccctttactgagaaaaaaaaaactcaaccttgattttgttgtacctggttgaatttgttattgttgtgtatacagttaaaaaactcaagtcttgattttattgtttcccttttgtagtttgtatatacatagagctgaattggtgttctaattttggttgatttttatgtatacagtataaaatcgatcttagttttgttcattgatttgtatttgcacaaagttggaattttgcgtttgttattttgatgattgaaaccttttctgtatatacagctcgagatgaatatttgtactaataagtcgtcgtcaggagtgaagaaaggtgcatggactgaagaagaagatgttctattgaaaaaatgcatcgagaaatatggagaaggaaagtggcatcaagttcctcttagagctggtttgaatagatgcagaaagagctgcagattaaggtggctaaattatctaaggccacatataaagagaggagacttctcttttgatgaagtagatctcattttgaggcttcataagctgttaggcaacagatggtcacttattgctggtagacttcctggaaggacggcaaacgatgtcaaaaactactggaacagccatcttcgcaagaagttaattgctcctcatgatcaaaaggagagcaagcaaaaagcaaagaagatcaccatattcagacctcggcctcgaaccttctcaaagacaaatacttgtgttaaaagtaacacaaatactgtagataaggatattgaaggcagcagcgaaataattagattcaacgataatttgaagccaacaactgaagaattgacggatgatggaattcaatggtgggccgatttactagctaacaattacaacaataatgggattgaggaagctgataattcatcaccaactttgttgcatgaggaaatgccacttctcagttgatctagaaataacagagggcgcgcgagcggtggctactgatcgcctatgagttctgtgattctacttgtaatttcagaagtgttttcagtgtcttgttttctggaagtccgtctggtttttagtaacttttagctcaaaaatgtgtctgtacgatggtatttgtatgtttgtgggtcttttacatatacgcttgtaatcgatcaatgtagaatgctgtgtgccttttccgtcaacagcttatttagtgtttactctgtatacgtatatctaatatatagtactgattctttcatctggtgatttgttttcctaaagagattattatcatagctttaattgaatgatacaaagaggtgttgcctggcttcaccagagcagaaattttcattgatatagggtacaaatgtcattcacataatgttaagagataagtttttcaatgtcctcaagagcccaccaagagtttcttccgggaattgcttaaattatcttaaatttaaattgtaaaactaataagaacaagcaatataagagaattcgtaaaaaactttctagttcttgaacatctacccatgtgaattctcttttatttttgtacactataattaaattaacatggctaaaccaattatgccaactgaataaattatcaatattgataaacttcaggtttacaccatgacgtgtgcaattatgaataaactccaagtttattatgatatgagtttttatatcaataaacttctactttactgtgacattcttttgtttgtgtagtacaaattggagaataaagcgtgtaacttttcacatacatattaccccgctataagttgtagtaatagaagatattaaatctttcctttctttatagtctcaatataaccaatcgcttttgcgaatactttagaaactaaataagtttctttgagacttactacaacataatgccttattgataatcaattttgttccttcaaaatagtaataattggctcttccagagctctcaattaattttgtactaccacatattcatcaacatccatatggtgaatatctcttttaaagagaaagttataccattatggttatggtcaaaattatatgcaatatttgtttcttgcattaccgttgccttttagtaatcacattgccaaaatattttggcctacaataagtacgtgaccaatgaccatttatgccataatgacattattttcttattttctctcaaacctccttcaagagatgagtgtggtatatactactagcacgctcatattcttccaaaaatgaatatgtattcataaatcacatattgtcacattcacatcatgaatggaccataatcatctatatgtgctttacaaaatctcatgtcaaatcgatgataaatattactttcacatagtgaatgattattttgagaatacttattattctcattaccacaatgaaaatgattataatttcgtctattgtcacatccacatccattgatacattcatggtaataattttatcttctttcagacgtatcatatattgctatcacattctcgtaagggaatgagaataaagcaaattcaatgggacgagtttccactttttgtgctcacaatcatacatgaatttgatcatggcaatacatagaaattttatcacttttggtggttataatttcttacaaactctattagagttataataaaaatttattgtctttgcttgtatttaaaatcaaattataaaattttaaaaattcaaaagagaaaaataaagtaaaatacttactttaaatccagaatttaatcacgaaggaagttcatggaataattgacgatcattatgctcaatcccaaagcttctactcaattggttacagtctcgtgccgataacgtgttataaaacaataaaagaagaacaatattgcagagaaagagagagagggaattcttattgaattttaggatgaattacaatggaataggacccctctatttatagggaaagagtgacttagccaccaagtaaaatccctaaaatctctctaaaatatagacattcaccttaaataaaactctatttataacaaaaacaaaattgttctagacgagaatcattttcactttttaatgtttttcttctactcctttagtttttccttaaaccaaaataaaaaaaaagtctatatttttctatcctttttcttctctagttttaatacattccataaaatttgtggtgaaattgtactttctccttttttttcctctttagtttcatagaaattaaacaataaaacgaaaacatcaaaatatatcaacaaatagtaaatttcattacatttatttttttgctcaaagtgctcttttgttgaatactcaaagtatatagtatattacagttctaatcaggaattattcactgaaatattgactgattttaggttcgtgtgatcgattaaatacctcttgaaccactacaaatttaacgaattaataacttttacctaacattttgttacccatgtattaatttgaatttatactattacttcctatttatgagtaatataacataatttctgtgtaaaggacagaaaatacagaatttgattaattcaaaattaattatacttcaaaaaatatcacaaggatatcaaaatttatcaaataaaggagcaacgtcattttcggctctcccacacaattcaaaataattcaaaacagaaccggctagacctaacctaaccaaaccatggttaatacaaatctaaatcaccgaaaaacaatcttaaccatagttaataacccgtattaattaacccgatatccatttcgccttgcccttattcattctcctataaaacagaggcccttcagtatagcgaagactcacaattcgaatttcgaaaactcatctctttctgttttaacggcgtcttgataaacgccgctccttctctcctcctctttgaaatttgaatttttagggtttcgtgaaactcgagaacaatgaatatttgtactaataagtcgtcgtcaggagtgaagaaaggtgcatggactgaagaagaagatgttctattgaaaaaatgcatcgagaaatatggagaaggaaagtggcatcaagttcctcttagagctggtttgaatagatgcagaaagagctgcagattaaggtggctaaattatctaaggccacatataaagagaggagacttctcttttgatgaagtagatctcattttgaggcttcataagctgttaggcaacagatggtcacttattgctggtagacttcctggaaggacggcaaacgatgtcaaaaactactggaacagccatcttcgcaagaagttaattgctcctcatgatcaaaaggagagcaagcaaaaagcaaagaagatcaccatattcagacctcggcctcgaaccttctcaaagacaaatacttgtgttaaaagtaacacaaatactgtagataaggatattgaaggcagcagcgaaataattagattcaacgataatttgaagccaacaactgaagaattgacggatgatggaattcaatggtgggccgatttactagctaacaattacaacaataatgggattgaggaagctgataattcatcaccaactttgttgcatgaggaaatgccacttctcagttgatctagaaataacagagggcgcgcgagcggtggctactgatcgcctatgagttctgtgattctacttgtaatttcagaagtgttttcagtgtcttgttttctggaagtccgtctggtttttagtaacttttagctcaaaaatgtgtctgtacgatggtatttgtatgtttgtgggtcttttacatatacgcttgtaatcgatcaatgtagaatgctgtgtgccttttccgtcaacagcttatttagtgtttactctgtatacgtatatctaatatatagtactgattctttcatctggtgatttgttttcctaaagagattattatcatagctttaattgaatgatacaaagaggtgttgcctggcttcaccagagcagaaattttcattgatatagggtacaaatgtcattcacataatgttaagagataagtttttcaatgtcctcaagagcccaccaagagtttcttccgggaattgcttaaattatcttaaatttaaattgtacccgtttaaac

SEQ ID NO: 63 - DNA, 7583 bp, synthetic construct
  misc_feature 1..7583: note = Chimera: comprises plant native sequences only
  source 1..7583: mol_type = other DNA
cccgggttagtattcaaaccgaataaatcaaagttaccaaaccgaataaatcgaaaccgaaaggagaaaccgcaccataccgaatttaattaggtacgatattgatattgtattttaaaaaatcgaataccaaaaataccgaattaaaatatctaaatatcgtacagtaccgaccgataaatactctataagcatcatcatgtcaccattcctgcttggaaatagatgtaataatgtaattcaaggtgaagatcattgaaaatgagatatttggactcttagataattgtgcaactgatatttttatttactttttttctttcatccaataattgcgttacattaaaaatgagatatttggattaatattcttctccttgaccacaaagcaaggaaagcctaaggaccgatagtaaagttgtattcgtgtggttgcgtgttagttttgagcggcaaaataaattatgttaaggtaaattatttttggaacaataataaaattatttctgtataatatataaatcatatatttgaaccgtagaattatcagttaatacttgtatatgaggaggctaactacgttagagcgctaacgagaatacttcatataccgtattttttacgataataataatgtaatgtgaaattgctatccaaaaggcacctaattttgtccaccgttcaaaggaaaggacaaggaagtagtagcgtgtaggtttggtgctgtacaaaataagcaagacacgtgttgccttattataggataatccataaggcaatttcgtcttaagtcggccattgcacctttaaaaggagcctctttgttcccaaaatcttcatcctttgatttctctattctcaatatctcctcaatttttctctagtcttcaaacacttctcaaggtacattaacttcttctttctttttgttcctcttattttatgctacttttatttaatttcgatctatatttttaggatctaaatactcatttttgatttgtttaatcgctctgtatatatgcaccaagttgaaatttttgtaagtttattttgttcggtctatattttaagatctgaaataccctttactgagaaaaaaaaaactcaaccttgattttgttgtacctggttgaatttgttattgttgtgtatacagttaaaaaactcaagtcttgattttattgtttcccttttgtagtttgtatatacatagagctgaattggtgttctaattttggttgatttttatgtatacagtataaaatcgatcttagttttgttcattgatttgtatttgcacaaagttggaattttgcgtttgttattttgatgattgaaaccttttctgtatatacagctcgagatgacggagataccgcctaacagccagatgaaaaccatgttgcagaaggcagtgcaatcggttcaatggacatatactcttttctggcaattatgtccccaacaaggggcgttagtgtggagagatggatattacaatggggctataaagactagaaagacagtgcagccaatggaagttagcgctgaggaagcttctcttcacagaagccaacagcttagagaactttacgaatcactttccgccggcgagtcaaatcagccagcgagaaggccgtcggcagctttgtcaccggaggacttgacggagtccgagtggttttatctcatgtgtgtttctttctcttttcctcctggcatcggattacctggcaaggcttattcgaagaaacatcacatatggatcatgggcgcaaacgaggttgatagcaaagtcttctgtagagctattcttgccaagagcgcccgcatacagacggtcgttggtattcctctcttggatggtgtactggaactgggaactacagaaagggttcaagaagagattggattcataaaccatgtaaagagctttttcactgagcaacaacaacctcagctaccaaagccagccttatctgagcactccacttccaatcccaccaccttttccgagccacatttttactccggcaatacttcgccatctgctaatgttgatattgcgcatcaagatggcggagctgccggcgaagaagatgaggaggaggaagaagaagaagatgatgatgaagccgagttggactcggatagtatagcgattcaaagcgcggctaatcctattgccgttgaggctagtgaactcatgcagcttgatgtgtccgaggctatacagctcggctcgcccgatgatgactctgataatatggactctgattttcatttggttggcgctggaaacacggctcatgactaccagcgccaagctgactctttcaaagccgagaccgccattagctggccgcacttccaagaccttcaacaattaccaggtggctctagttatgatgaattatcacaagaagacacacactattctcaaacagtgtcaaccattctcgaacaccgaagctccaaattttcctctacaacaatgggctgtatttctcatgactcggcccaatctgccttcacattgtgccctagcaccaccgtctgcagcccgaatcccgcccactgccgccacgacgactcacttgtcgacggtggcggcgcctcccagtggctgctcaaaagcatactcttcactgtcccatttcttcacactaaataccaatctgaagcttctccaaagtcacgtgacgtcgccactgttgattcctccagtactgcttctcgctttcgcaaaggctgtagtataacgtcgcaagaagagccaagtggaaaccatgtacttgcagaacgacgtcgtagagagaagctaaatgagcgttttatcatattaaggtctcttgtaccttttgtaacgaaaatggacaaagcctccattttgggtgacaccatagagtatgtcaagcagttacgtaagaaagttcaggatcttgaagctcgtgctcgcgacacggagcactccagagatgcagataaaaaaggtggcacagctacagtgaaggtgttgcaaggaaggggtaagaggagaatgaatacggtagatggaagtgttggtggagggcaggcaacgataacggcgtccccaccgtcaacgacggaaaatgaggaggttgtgcaagtacaagtatcaattatcgaaagcgatgcattggtggagctccggtgtccgtacaaagaggggttgctgttaaatgtaatgcagatgctaagggaactcaaagtggaagttgtagccattcaatcagctcttaataatggcgtcttcttggctgagttaagagctaaggtaaaagagaatatatgtggaaggaaagcaagcattttggaagtaaaaaggtcaatacatcagataatccctagagattaatctagaaataacagagggcgcgcgagcggtggctactgatcgcctatgagttctgtgattctacttgtaatttcagaagtgttttcagtgtcttgttttctggaagtccgtctggtttttagtaacttttagctcaaaaatgtgtctgtacgatggtatttgtatgtttgtgggtcttttacatatacgcttgtaatcgatcaatgtagaatgctgtgtgccttttccgtcaacagcttatttagtgtttactctgtatacgtatatctaatatatagtactgattctttcatctggtgatttgttttcctaaagagattattatcatagctttaattgaatgatacaaagaggtgttgcctggcttcaccagagcagaaattttcattgatatagggtacaaatgtcattcacataatgttaagagataagtttttcaatgtcctcaagagcccaccaagagtttcttccgggaattgcttaaattatcttaaatttaaattgtaaaactaataagaacaagcaatataagagaattcgtaaaaaactttctagttcttgaacatctacccatgtgaattctcttttatttttgtacactataattaaattaacatggctaaaccaattatgccaactgaataaattatcaatattgataaacttcaggtttacaccatgacgtgtgcaattatgaataaactccaagtttattatgatatgagtttttatatcaataaacttctactttactgtgacattcttttgtttgtgtagtacaaattggagaataaagcgtgtaacttttcacatacatattaccccgctataagttgtagtaatagaagatattaaatctttcctttctttatagtctcaatataaccaatcgcttttgcgaatactttagaaactaaataagtttctttgagacttactacaacataatgccttattgataatcaattttgttccttcaaaatagtaataattggctcttccagagctctcaattaattttgtactaccacatattcatcaacatccatatggtgaatatctcttttaaagagaaagttataccattatggttatggtcaaaattatatgcaatatttgtttcttgcattaccgttgccttttagtaatcacattgccaaaatattttggcctacaataagtacgtgaccaatgaccatttatgccataatgacattattttcttattttctctcaaacctccttcaagagatgagtgtggtatatactactagcacgctcatattcttccaaaaatgaatatgtattcataaatcacatattgtcacattcacatcatgaatggaccataatcatctatatgtgctttacaaaatctcatgtcaaatcgatgataaatattactttcacatagtgaatgattattttgagaatacttattattctcattaccacaatgaaaatgattataatttcgtctattgtcacatccacatccattgatacattcatggtaataattttatcttctttcagacgtatcatatattgctatcacattctcgtaagggaatgagaataaagcaaattcaatgggacgagtttccactttttgtgctcacaatcatacatgaatttgatcatggcaatacatagaaattttatcacttttggtggttataatttcttacaaactctattagagttataataaaaatttattgtctttgcttgtatttaaaatcaaattataaaattttaaaaattcaaaagagaaaaataaagtaaaatacttactttaaatccagaatttaatcacgaaggaagttcatggaataattgacgatcattatgctcaatcccaaagcttctactcaattggttacagtctcgtgccgataacgtgttataaaacaataaaagaagaacaatattgcagagaaagagagagagggaattcttattgaattttaggatgaattacaatggaataggacccctctatttatagggaaagagtgacttagccaccaagtaaaatccctaaaatctctctaaaatatagacattcaccttaaataaaactctatttataacaaaaacaaaattgttctagacgagaatcattttcactttttaatgtttttcttctactcctttagtttttccttaaaccaaaataaaaaaaaagtctatatttttctatcctttttcttctctagttttaatacattccataaaatttgtggtgaaattgtactttctccttttttttcctctttagtttcatagaaattaaacaataaaacgaaaacatcaaaatatatcaacaaatagtaaatttcattacatttatttttttgctcaaagtgctcttttgttgaatactcaaagtatatagtatattacagttctaatcaggaattattcactgaaatattgactgattttaggttcgtgtgatcgattaaatacctcttgaaccactacaaatttaacgaattaataacttttacctaacattttgttacccatgtattaatttgaatttatactattacttcctatttatgagtaatataacataatttctgtgtaaaggacagaaaatacagaatttgattaattcaaaattaattatacttcaaaaaatatcacaaggatatcaaaatttatcaaataaaggagcaacgtcattttcggctctcccacacaattcaaaataattcaaaacagaaccggctagacctaacctaaccaaaccatggttaatacaaatctaaatcaccgaaaaacaatcttaaccatagttaataacccgtattaattaacccgatatccatttcgccttgcccttattcattctcctataaaacagaggcccttcagtatagcgaagactcacaattcgaatttcgaaaactcatctctttctgttttaacggcgtcttgataaacgccgctccttctctcctcctctttgaaatttgaatttttagggtttcgtgaaactcgagaacaatgaatatttgtactaataagtcgtcgtcaggagtgaagaaaggtgcatggactgaagaagaagatgttctattgaaaaaatgcatcgagaaatatggagaaggaaagtggcatcaagttcctcttagagctggtttgaatagatgcagaaagagctgcagattaaggtggctaaattatctaaggccacatataaagagaggagacttctcttttgatgaagtagatctcattttgaggcttcataagctgttaggcaacagatggtcacttattgctggtagacttcctggaaggacggcaaacgatgtcaaaaactactggaacagccatcttcgcaagaagttaattgctcctcatgatcaaaaggagagcaagcaaaaagcaaagaagatcaccatattcagacctcggcctcgaaccttctcaaagacaaatacttgtgttaaaagtaacacaaatactgtagataaggatattgaaggcagcagcgaaataattagattcaacgataatttgaagccaacaactgaagaattgacggatgatggaattcaatggtgggccgatttactagctaacaattacaacaataatgggattgaggaagctgataattcatcaccaactttgttgcatgaggaaatgccacttctcagttgatctagaaataacagagggcgcgcgagcggtggctactgatcgcctatgagttctgtgattctacttgtaatttcagaagtgttttcagtgtcttgttttctggaagtccgtctggtttttagtaacttttagctcaaaaatgtgtctgtacgatggtatttgtatgtttgtgggtcttttacatatacgcttgtaatcgatcaatgtagaatgctgtgtgccttttccgtcaacagcttatttagtgtttactctgtatacgtatatctaatatatagtactgattctttcatctggtgatttgttttcctaaagagattattatcatagctttaattgaatgatacaaagaggtgttgcctggcttcaccagagcagaaattttcattgatatagggtacaaatgtcattcacataatgttaagagataagtttttcaatgtcctcaagagcccaccaagagtttcttccgggaattgcttaaattatcttaaatttaaattgtacccgtttaaac

(19) United States
(12) Patent Application Publication
(10) Pub. No.: US 2025/0011803 A1
(43) Pub. Date: Jan. 9, 2025

(54) Composition and Methods for Producing Tobacco Plants and Products Having Reduced Tobacco-Specific Nitrosamines (TSNAs)

(71) Applicant: Altria Client Services LLC, Richmond, VA (US)

(72) Inventors: Chengalrayan KUDITHIPUDI, Midlothian, VA (US); Dongmei XU, Glen Allen, VA (US); James Strickland, Richmond, VA (US)

(73) Assignee: Altria Client Services LLC, Richmond, VA (US)

(21) Appl. No.: 18/896,070

(22) Filed: Sep. 25, 2024

Related U.S. Application Data
(63) Continuation of application No. 18/182,701, filed on Mar. 13, 2023, now Pat. No. 12,129,474.
Application No. 18/182,701 is a continuation of application No. 16/838,762, filed on Apr. 2, 2020, now Pat. No. 11,634,724.
Application No. 16/838,762 is a continuation of application No. 15/727,523, filed on Oct. 6, 2017, now Pat. No. 10,647,989.
(60) Provisional application No. 62/405,607, filed on Oct. 7, 2016.
(60) Provisional application No. 62/503,103, filed on May 8, 2017.

(51) Int. Cl. C12N 15/82; A01H 4/00; A24B 13/00; A24B 15/10; A24B 15/24; B01F 33/45; B01F 33/452; B01F 33/501; B01F 35/75; B01L 3/00; C07K 14/415; C12N 9/04; G01N 1/38; G01N 35/00; G01N 35/10; H02N 11/00

(52) U.S. Cl. CPC C12N 15/8243; A01H 4/005; A01H 4/008; A24B 13/00; A24B 15/10; A24B 15/245; B01F 33/45; B01F 33/452; B01F 33/50112; B01F 35/754251; B01L 3/502; B01L 3/50825; C07K 14/415; C12N 9/0006; C12N 15/8205; C12N 15/8225; C12N 15/825; G01N 1/38; H02N 11/006; B01L 2200/0684; B01L 2300/043; B01L 2300/0672; B01L 2300/0867; B01L 2400/0403; B01L 2400/043; B01L 2400/0478; B01L 2400/0683; G01N 2035/00534; G01N 35/1079

(57) ABSTRACT

The present disclosure provides approaches for reducing tobacco-specific nitrosamines (TSNAs) in tobacco. Some of these approaches include genetically engineering tobacco plants to increase one or more antioxidants, increase oxygen radicle absorbance capacity (ORAC), or reduce nitrite. Also provided are methods and compositions for producing modified tobacco plants and tobacco products therefrom comprising reduced TSNAs.

### CROSS-REFERENCE TO RELATED APPLICATIONS

This application is a Continuation of U.S. patent application Ser. No. 18/182,701, filed Mar. 13, 2023, which is a Continuation of U.S. patent application Ser. No. 16/838,762, filed Apr. 2, 2020 (now U.S. Pat. No. 11,634,724, issued: Apr. 25, 2023), which is a Continuation of U.S. patent application Ser. No. 15/727,523, filed Oct. 6, 2017 (now U.S. Pat. No. 10,647,989, issued: May 12, 2020), which claims priority to U.S. Provisional Application No. 62/405,607, filed Oct. 7, 2016, and U.S. Provisional Application No. 62/503,103, filed May 8, 2017. Each of these applications are incorporated by reference herein in their entireties.

### INCORPORATION OF SEQUENCE LISTING

A sequence listing contained in the file named “P34473US04_ST26.xml” which is 131,184 bytes (measured in MS-Windows®) and created on Mar. 31, 2023, comprises 63 sequences, is filed electronically herewith and incorporated by reference in its entirety.

### FIELD

The present disclosure relates generally to methods for reducing tobacco specific nitrosamines (TSNAs) comprising modulating the levels of antioxidants, nitrite, or oxygen radical absorbance capacity (ORAC). Also provided are methods and compositions related to reducing or eliminating TSNAs in cured leaf from tobacco plants and products, their development via breeding or transgenic approaches, and production of tobacco products from those tobacco plants.

### BACKGROUND

Tobacco-specific nitrosamines (TSNAs), such as N-nitrosonornicotine (NNN) and 4-(methylnitrosamino)-1-(3-pyridyl)-1-butanone (NNK), can be found in smokeless tobacco; mainstream smoke; and side stream smoke of cigarettes. It has been reported that air-cured and flue-cured tobacco contain tobacco-specific nitrosamines. See, “Effect of Air-Curing on the Chemical Composition of Tobacco”, Wiernik et al., Recent Adv. Tob. Sci, (1995), 21, pp. 39-80. According to Wiernik et al., TSNAs are not present in significant quantities in growing tobacco plants or fresh cut tobacco (green tobacco), but are formed during the curing process. Bacterial populations which reside on the tobacco leaf are stated to largely cause the formation of nitrites from nitrate during curing and possibly affect the direct catalysis of the nitrosation of secondary amines at physiological pH values. The affected secondary amines include tobacco alkaloids, which form TSNAs when nitrosated.

Prior reports suggest several approaches to reduce TSNA levels. For example, WO2003/022081 proposed methods for reducing tobacco-specific nitrosamine (TSNA) content in cured tobacco by increasing the levels of antioxidants in the tobacco prior to harvesting. Specifically, WO2003/022081 proposed root pruning of the tobacco plant prior to harvesting; severing the xylem tissue of the tobacco plant prior to harvesting; and administering antioxidants and/or chemicals which increase antioxidants to the tobacco plant after harvesting. Despite previous attempts and proposals, simpler, more uniform, more economical and non-labor-intensive methods are desirable for reducing TSNA levels in cured tobacco leaf. Here, the inventors address this need by providing methods and compositions for reducing TSNAs by manipulating antioxidant levels via, inter alia, modification of genes involved in antioxidant biosynthesis or regulation thereof.

### SUMMARY

In one aspect, the present disclosure provides cured tobacco leaf from a modified tobacco plant described here, where the cured tobacco leaf comprises a reduced level of one or more tobacco-specific nitrosamines (TSNAs) and further comprises an increased level of one or more antioxidants, wherein said reduced and increased levels are compared to cured tobacco leaf from a control tobacco plant of the same variety when grown and cured under comparable conditions.

In another aspect, the present disclosure provides a tobacco product comprising or made from cured leaf from a modified tobacco plant described here.

In one aspect, the present disclosure provides cured tobacco leaf from a modified tobacco plant comprising a reduced level of one or more tobacco-specific nitrosamines (TSNAs) and further comprising an increased level of one or more antioxidants, wherein said reduced and increased levels are compared to cured tobacco leaf from a control tobacco plant of the same variety when grown and cured under comparable conditions.

In another aspect, the present disclosure provides cured tobacco leaf from a modified tobacco plant comprising a reduced level of one or more tobacco-specific nitrosamines (TSNAs) and further comprising a reduced level of nitrite, wherein said reduced levels are compared to cured tobacco leaf from a control tobacco plant of the same variety when grown and cured under comparable conditions.

In one aspect, the present disclosure provides a modified tobacco plant or cured tobacco leaf from a modified tobacco plant comprising a reduced level of one or more tobacco-specific nitrosamines (TSNAs) and further comprising an increased level of oxygen radical absorbance capacity (ORAC), and wherein said reduced and increased levels are compared to a control tobacco plant or cured tobacco leaf from a control tobacco plant of the same variety when grown and cured under comparable conditions.

In another aspect, the present disclosure provides a seed giving rise to a modified tobacco plant described here.

In one aspect, the present disclosure provides a method comprising: planting a seed; and growing from the seed a modified tobacco plant described here.

In another aspect, the present disclosure provides a method comprising preparing a tobacco product using cured tobacco leaf from a modified tobacco plant described here.

In one aspect, the present disclosure provides a method of reducing the level of one or more TSNAs in cured tobacco leaf from a tobacco plant, said method comprising increasing the level of one or more antioxidants in said tobacco plant by expressing a biosynthetic enzyme, a regulatory transcription factor, a transporter, a catabolic enzyme, or a combination thereof, for said one or more antioxidants.

In another aspect, the present disclosure provides a method for producing a tobacco plant comprising: crossing at least one tobacco plant of a first tobacco variety with at least one tobacco plant of a second tobacco variety, wherein said at least one tobacco plant of said first tobacco variety is a modified tobacco plant described here; and selecting for a progeny tobacco plant capable of producing cured tobacco leaf comprising reduced levels of one or more tobacco-specific nitrosamines (TSNAs) and further comprising one or more traits selected from the group consisting of: a reduced level of nitrite, an increased level of oxygen radical absorbance capacity (ORAC), and an increased level of one or more antioxidants; wherein said reduced or increased level is compared to a control tobacco plant or cured tobacco leaf from a control tobacco plant of the same cross grown and cured under comparable conditions.

In one aspect, the present disclosure provides a method for reducing the level of one or more TSNAs in cured tobacco leaf or a tobacco product made therefrom, the method comprising increasing the level of one or more antioxidants in a tobacco plant via a transgene encoding or directly modulating an antioxidant biosynthetic enzyme, a regulatory transcription factor of an antioxidant, an antioxidant transporter, an antioxidant metabolic enzyme, or a combination thereof; and reducing the level of one or more TSNAs in cured tobacco leaf from the tobacco plant or a tobacco product made from the cured tobacco leaf.

In one aspect, the present disclosure provides a method for reducing the level of one or more TSNAs in cured tobacco leaf or a tobacco product made therefrom, the method comprising increasing the level of one or more antioxidants in a tobacco plant via a genetic modification in an endogenous gene, wherein the endogenous gene encodes an antioxidant biosynthetic enzyme, a regulatory transcription factor of an antioxidant, an antioxidant transporter, an antioxidant metabolic enzyme, or a combination thereof; and reducing the level of one or more TSNAs in cured tobacco leaf from the tobacco plant or a tobacco product made from the cured tobacco leaf.

In one aspect, the present disclosure provides a method for manufacturing a tobacco product, the method comprising obtaining cured tobacco leaf comprising a transgene or comprising a genetic modification in an endogenous gene, and further comprising an increased level of one or more antioxidants compared to a control cured tobacco leaf lacking the transgene or the genetic modification, wherein an endogenous gene encodes an antioxidant biosynthetic enzyme, a regulatory transcription factor of an antioxidant, an antioxidant transporter, an antioxidant metabolic enzyme, or a combination thereof, wherein the transgene encodes or directly modulates an antioxidant biosynthetic enzyme, a regulatory transcription factor of an antioxidant, an antioxidant transporter, an antioxidant metabolic enzyme, or a combination thereof; and producing a tobacco product from cured tobacco leaf, wherein the tobacco product comprises a reduced level of one or more TSNAs relative to a control tobacco product prepared from a control cured tobacco leaf.

In one aspect, the present disclosure provides a method for preparing cured tobacco leaf, the method comprising growing a tobacco plant comprising a transgene or a genetic modification in an endogenous gene, and further comprising an increased level of one or more antioxidants compared to a control tobacco plant lacking the transgene or the genetic modification, wherein an endogenous gene encodes an antioxidant biosynthetic enzyme, a regulatory transcription factor of an antioxidant, an antioxidant transporter, an antioxidant metabolic enzyme, or a combination thereof, wherein the transgene encodes or directly modulates an antioxidant biosynthetic enzyme, a regulatory transcription factor of an antioxidant, an antioxidant transporter, an antioxidant metabolic enzyme, or a combination thereof; and preparing cured leaf from a tobacco plant, wherein cured leaf comprises a reduced level of one or more TSNAs relative to a control cured leaf from a control tobacco plant not comprising the transgene or the genetic modification.

In one aspect, the present disclosure provides cured leaf of a modified tobacco plant, wherein cured leaf comprises a reduced level of one or more tobacco-specific nitrosamines (TSNAs) and further comprises an increased level of one or more antioxidants and a reduced nitrite level, wherein reduced and increased levels are compared to a control cured leaf of an unmodified tobacco plant of the same variety when grown and cured under comparable conditions, wherein the modification comprises a transgene or a genetic modification in an endogenous gene, wherein a transgene or an endogenous gene encodes an antioxidant biosynthetic enzyme, a regulatory transcription factor of an antioxidant, an antioxidant transporter, an antioxidant metabolic enzyme, or a combination thereof; wherein the modified tobacco plant does not comprise a transgene overexpressing an Arabidopsis PAP1 protein.

### BRIEF DESCRIPTION OF THE SEQUENCES

SEQ ID NOs: 1 to 23 and 47 to 52 are amino acid sequences of selected genes that are involved in antioxidant production. SEQ ID NOs: 24 to 46 and 53 to 58 are corresponding nucleic acid sequences that encode SEQ ID NOs: 1 to 23, and 47 to 52. SEQ ID NOs: 59 to 63 are polynucleotides encoding recombinant DNA molecules comprising cisgenic promoters, coding regions, and terminators.

### BRIEF DESCRIPTION OF THE DRAWINGS

FIG. 1: TSNAs are formed when alkaloids nitrosinate in the presence of nitrite.

FIG. 2: Cloning of AtPAP1, NtAN2, and NtAN1 into 45-2-7 binary vector.

FIG. 3: A control plant (left) exhibits a similar growth profile compared AtPAP1 overexpression plants (right). AtPAP1 plants exhibit a purple color due to anthocyanin accumulation.

FIGS. 4A-4E: TSNA reduction in five AtPAP1 overexpression lines. FIG. 4A: total TSNAs are reduced in AtPAP1 overexpression lines. FIG. 4B: NNN levels are reduced in AtPAP1 overexpression lines compared to controls. FIG. 4C: NNK levels are reduced in AtPAP1 overexpression lines compared to controls. FIG. 4D: NAB levels are reduced in AtPAP1 overexpression lines compared to controls. FIG. 4E: NAT levels are reduced in AtPAP1 overexpression lines compared to controls.

FIG. 5: Oxygen radical absorbance capacity (ORAC) values in AtPAP1 overexpression plants are increased compared to controls.

FIGS. 6A-6B: Nitrite and Nitrate levels in AtPAP1 overexpression plants. FIG. 6A: Nitrite levels in AtPAP1 overexpression plants are reduced compared to controls. FIG. 6B: Nitrate levels in AtPAP1 overexpression plants are not consistently different from controls.

FIG. 7: HCT and HQT function in the biosynthetic pathway of Chlorogenic Acid.

FIG. 8: Chlorogenic Acid levels are reduced in 3 of 4 HQT RNAi lines but not in HCT RNAi lines.

FIG. 9: Total TSNAs are increased in the 3 HQT RNAi lines with decreased Chlorogenic Acid levels.

FIG. 10: Accumulation of Chlorogenic Acid is inversely correlated with TSNA levels. A negative correlation is observed between CGA levels and total TSNA levels as shown in Table 4 and FIG. 10A. This correlation is also observed between CGA levels and individual TSNAs NNN (FIG. 10B), NNK (FIG. 10C), NAB (FIG. 10D), and NAA (FIG. 10E).

FIG. 11: The phenylpropanoid pathway can be targeted to reduce TSNA levels in tobacco by increasing antioxidant levels.

FIG. 12: The phenylpropanoid pathway leads to the biosynthesis of many antioxidants.

FIG. 13: Overexpression of AtPAP1 in TN90 and Narrow Leaf Madole (NLM) results in increased antioxidant capacity as measured using a FRAP assay. Measurements from leaves from at least five plants are averaged together for unmodified TN90 and NLM, two independent lines overexpressing AtPAP1 in TN90, and two independent lines overexpressing AtPAP1 in NLM are tested using a FRAP assay. Both independent lines overexpressing AtPAP1 in TN90 exhibit a highly significant increase in average antioxidant capacity (P<0.01) compared to unmodified TN90 plants. Both independent lines overexpressing AtPAP1 in NLM exhibit a highly significant increase in average antioxidant capacity (P<0.01) compared to unmodified NLM plants.

FIG. 14: Overexpression of NtAN2 (SEQ ID NO: 30) in NLM results in increased antioxidant capacity as measured using a FRAP assay. Greenhouse grown, individually tested TO plants overexpressing NtAN2 show increased antioxidant capacity compared to the average antioxidant capacity determined for at least five unmodified NLM plants.

FIG. 15: Overexpression of NtAN1a (SEQ ID NO: 28) in NLM results in increased antioxidant capacity as measured using a FRAP assay. Greenhouse grown, individually tested TO plants overexpressing NtAN1a show increased antioxidant capacity compared to the average antioxidant capacity determined for at least five unmodified NLM plants.

FIG. 16: Overexpression of NtDFR (SEQ ID NO: 37) in NLM results in increased antioxidant capacity as measured using a FRAP assay. Greenhouse grown, individually tested TO plants overexpressing NtDFR show increased antioxidant capacity compared to the average antioxidant capacity determined for at least five unmodified NLM plants.

FIG. 17: Overexpression of NtJAF13 (SEQ ID NO: 33) in NLM results in increased antioxidant capacity as measured using a FRAP assay. Greenhouse grown, individually tested TO plants overexpressing NtJAF13 show increased antioxidant capacity compared to the average antioxidant capacity determined for at least five unmodified NLM plants.

FIG. 18: Overexpression of NtMYB3 (SEQ ID NO: 36) in NLM results in increased antioxidant capacity as measured using a FRAP assay. Greenhouse grown, individually tested TO plants overexpressing NtMYB3 show increased antioxidant capacity compared to the average antioxidant capacity determined for at least five unmodified NLM plants.

FIG. 19: Overexpression of NtMYB3 (SEQ ID NO: 36) in NLM results in tobacco plants with normal leaf color in T0 plants grown in the greenhouse.

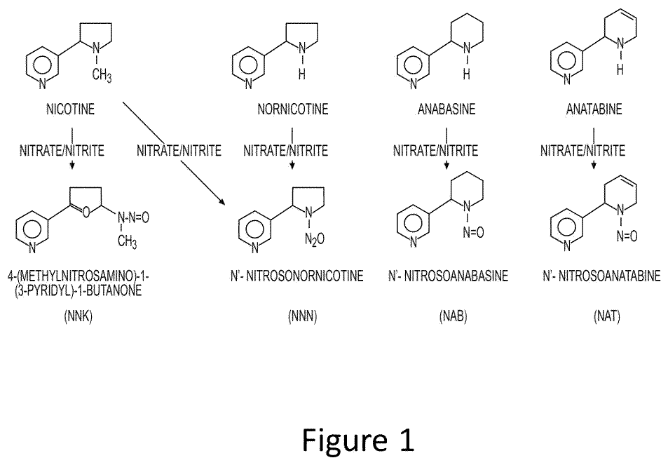

### DETAILED DESCRIPTION

Unless defined otherwise, technical and scientific terms used herein have the same meaning as commonly understood by one of ordinary skill in the art. One skilled in the art will recognize many methods can be used in the practice of the present disclosure. Indeed, the present disclosure is in no way limited to the methods and materials described. For purposes of the present disclosure, the following terms are defined below.

Any references cited herein, including, e.g., all patents, published patent applications, and non-patent publications, are incorporated by reference in their entirety.

As used herein, the singular form “a,” “an,” and “the” include plural references unless the context clearly dictates otherwise. For example, the term “a compound” or “at least one compound” may include a plurality of compounds, including mixtures thereof.

The term “about” is used herein to mean approximately, roughly, around, or in the region of. When the term “about” is used in conjunction with a numerical range, it modifies that range by extending the boundaries above and below the numerical values set forth by 10%.

As used herein, a tobacco plant can be from any plant from the Nicotiana tabacum genus including, but not limited to Nicotiana tabacum tabacum; Nicotiana tabacum amplexicaulis PI 271989; Nicotiana tabacum benthamiana PI 555478; Nicotiana tabacum bigelovii PI 555485; Nicotiana tabacum debneyi; Nicotiana tabacum excelsior PI 224063; Nicotiana tabacum glutinosa PI 555507; Nicotiana tabacum goodspeedii PI 241012; Nicotiana tabacum gossei PI 230953; Nicotiana tabacum hesperis PI 271991; Nicotiana tabacum knightiana PI 555527; Nicotiana tabacum maritima PI 555535; Nicotiana tabacum megalosiphon PI 555536; Nicotiana tabacum nudicaulis PI 555540; Nicotiana tabacum paniculata PI 555545; Nicotiana tabacum plumbaginifolia PI 555548; Nicotiana tabacum repanda PI 555552; Nicotiana tabacum rustica; Nicotiana tabacum suaveolens PI 230960; Nicotiana tabacum sylvestris PI 555569; Nicotiana tabacum tomentosa PI 266379; Nicotiana tabacum tomentosiformis; and Nicotiana tabacum trigonophylla PI 555572.

In one aspect, this disclosure provides methods and compositions related to modified tobacco plants, seeds, plant components, plant cells, and products made from modified tobacco plants, seeds, plant parts, and plant cells. In one aspect, a modified seed provided herein gives rise to a modified plant provided herein. In one aspect, a modified plant, seed, plant component, plant cell, or plant genome provided herein comprises a recombinant DNA construct provided herein. In another aspect, cured tobacco material or tobacco products provided herein comprise modified tobacco plants, plant components, plant cells, or plant genomes provided herein.

As used herein, “modified” refers to plants, seeds, plant components, plant cells, and plant genomes that have been subjected to mutagenesis, genome editing, genetic transformation, or a combination thereof.

In one aspect, the present disclosure provides a modified tobacco plant capable of producing cured tobacco leaf comprising a reduced level of one or more tobacco-specific nitrosamines (TSNAs) and further comprising an increased level of one or more antioxidants, wherein the reduced and increased levels are compared to a control tobacco plant or cured leaf from a control tobacco plant of the same variety when grown and cured under comparable conditions. In one aspect, a reduced level of one or more TSNAs is less than 50% of the level of the one or more TSNAs in cured leaf from a control plant. In another aspect, a modified tobacco plant further comprises an increased level of oxygen radical absorbance capacity (ORAC) compared to a control tobacco plant when grown and cured under comparable conditions. In a further aspect, cured leaf from a modified tobacco plant comprises a reduced level of nitrite compared to cured leaf from a control tobacco plant when grown and cured under comparable conditions.

In another aspect, cured leaf from a modified tobacco plant comprises a reduced level of total TSNAs compared to the cured leaf from a control tobacco plant when grown and cured under comparable conditions. In one aspect, reduced one or more TSNAs are selected from the group consisting of N′-nitrosonornicotine (NNN), 4-methylnitrosoamino-1-(3-pyridyl)-1-butanone (NNK), N′-nitrosoanatabine (NAT) N′-nitrosoanabasine (NAB), and any combination thereof. In one aspect, the level of total TSNAs or an individual TSNA is measured based on a freeze-dried cured leaf sample using liquid chromatograph with tandem mass spectrometry (LC/MS/MS).

In one aspect, the present disclosure provides cured leaf from a modified tobacco plant comprising a reduced level of 4-methylnitrosoamino-1-(3-pyridyl)-1-butanone (NNK) compared to cured leaf from a control tobacco plant of the same variety when grown and cured under comparable conditions. In one aspect, a reduced level of NNK is less than 50% of the level of the NNK in cured leaf from a control plant. In one aspect, a modified tobacco plant or cured leaf from a modified tobacco plant further comprises an increased level of one or more antioxidants compared to a control tobacco plant or cured tobacco leaf from a control plant of the same variety when grown and cured under comparable conditions. In another aspect, a modified tobacco plant or cured leaf from a modified tobacco plant further comprises an increased level of oxygen radical absorbance capacity (ORAC) compared to a control tobacco or cured tobacco leaf from a control plant when grown and cured under comparable conditions. In a further aspect, cured leaf from a modified tobacco plant comprises a reduced level of nitrite compared to cured leaf from a control tobacco plant when grown and cured under comparable conditions. The role of nitrite in the formation is nitrosamines and TSNAs is linked to the reduction of nitrate by the activity of bacteria during the curing process. Nitrite is believed to generate nitrosating compounds which then react with secondary amines such as the tobacco alkaloids nicotine, nornicotine, anabasine, and anatabine to form TSNAs. Reducing the amount of nitrite and therefore the nitrosation of tobacco alkaloids, the production of TSNAs can be prevented during the curing process.

In one aspect, a modified tobacco plant or cured leaf from a modified tobacco plant comprises an increased level of one or more antioxidants selected from the group consisting of anthocyanidin, flavanone, flavanol, flavone, flavonol, isoflavone, hydroxybenzoic acid, hydroxycinnamic acid, ellagitannin, stibene, lignan, carotenoids, and glycyrrhzin.

In another aspect, a modified tobacco plant or cured leaf from a modified tobacco plant comprises an increased level of one or more antioxidants selected from the group consisting of Delphnidin, Cyanidin, Procyanidin, Prodelphinidin, Hesperetin, Naringenin, Catechin, Epicatechin, Apigenin, Luteonin, Quercetin, Myricetin, Rutin, Genistein, Daidzein, Gallic acid, Vanillic acid, Protocatechuic acid, Ferunic acid, Cinnamic acid, Coumeric acid, Chlorogenic acid, Coffeic acid, ferulic acid, Sanguiin, Resveratrol, Sesamin, Caretonoids, and Vitamin C.

In one aspect, a modified tobacco plant or cured leaf from a modified tobacco plant comprises an antioxidant that is undetectable in the control plant or leaf. In another aspect, a modified tobacco plant or cured leaf from a modified tobacco plant comprises an antioxidant that does not exist in the control plant.

In another aspect, the present disclosure provides cured leaf from a modified tobacco plant comprising a reduced level of one or more tobacco-specific nitrosamines (TSNAs) and further comprising a reduced level of nitrite, wherein the reduced levels are compared to cured leaf from a control tobacco plant of the same variety when grown and cured under comparable conditions. In another aspect, a modified tobacco plant or cured leaf from a modified tobacco plant further comprises an increased level of oxygen radical absorbance capacity (ORAC) compared to the control tobacco plant or cured leaf from the control tobacco plant when grown and cured under comparable conditions.

In a further aspect, the present disclosure provides a modified tobacco plant capable of producing cured leaf comprising a reduced level of one or more tobacco-specific nitrosamines (TSNAs) and further comprising an increased level of oxygen radical absorbance capacity (ORAC), and wherein the reduced and increased levels are compared to a control tobacco plant or cured leaf from a control tobacco plant of the same variety when grown and cured under comparable conditions.

In one aspect, a reduced or increased level is within about 10%, within about 20%, within about 30%, within about 40%, within about 50%, within about 60%, within about 70%, within about 80%, within about 90%, within about 92%, within about 94%, within about 95%, within about 96%, within about 97%, within about 98%, or within about 99% lower or higher than the level in a control tobacco plant or cured leaf from a control tobacco plant when grown and cured under comparable conditions.

In another aspect, a reduced or increased level is within about 1 fold, within about 2 folds, within about 3 folds, within about 4 folds, within about 5 folds, within about 6 folds, within about 7 folds, within about 8 folds, within about 9 folds, within about 10 folds, within about 15 folds, within about 20 folds, within about 25 folds, or within about 30 folds lower or higher than the level in a control tobacco plant or cured leaf from a control tobacco plant when grown and cured under comparable conditions.

In one aspect, a reduced or increased level is at least about 10%, at least about 20%, at least about 30%, at least about 40%, at least about 50%, at least about 60%, at least about 70%, at least about 80%, at least about 90%, at least about 92%, at least about 94%, at least about 95%, at least about 96%, at least about 97%, at least about 98%, or at least about 99% lower or higher than the level in a control tobacco plant or cured leaf from a control tobacco plant when grown and cured under comparable conditions.

In another aspect, a reduced or increased level is at least about 1 fold, at least about 2 folds, at least about 3 folds, at least about 4 folds, at least about 5 folds, at least about 6 folds, at least about 7 folds, at least about 8 folds, at least about 9 folds, at least about 10 folds, at least about 15 folds, at least about 20 folds, at least about 25 folds, or at least about 30 folds lower or higher than the level in a control tobacco plant or cured leaf from a control tobacco plant when grown and cured under comparable conditions.

In one aspect, a reduced or increased level is about 10%, about 20%, about 30%, about 40%, about 50%, about 60%, about 70%, about 80%, about 90%, about 92%, about 94%, about 95%, about 96%, about 97%, about 98%, or about 99% lower or higher than the level in a control tobacco plant or cured leaf from a control tobacco plant when grown and cured under comparable conditions.

In another aspect, a reduced or increased level is about 1 fold, about 2 folds, about 3 folds, about 4 folds, about 5 folds, about 6 folds, about 7 folds, about 8 folds, about 9 folds, about 10 folds, about 15 folds, about 20 folds, about 25 folds, or about 30 folds lower or higher than the level in a control tobacco plant or cured leaf from a control tobacco plant when grown and cured under comparable conditions.

In one aspect, a reduced or increased level is about 1-2 folds, about 2-3 folds, about 3-4 folds, about 4-5 folds, about 5-6 folds, about 6-7 folds, about 7-8 folds, about 8-9 folds, about 9-10 folds, about 10-15 folds, about 15-20 folds, about 20-25 folds, about 25-30 folds, or about 30-50 folds lower or higher than the level in a control tobacco plant or cured leaf from a control tobacco plant when grown and cured under comparable conditions.

In another aspect, a reduced or increased level is about 1-10 folds, about 2-10 folds, about 3-10 folds, about 4-10 folds, about 5-10 folds, about 6-10 folds, about 7-10 folds, about 8-10 folds, about 9-10 folds, about 10-50 folds, about 15-50 folds, about 20-50 folds, about 25-50 folds, or about 30-50 folds lower or higher than the level in a control tobacco plant or cured leaf from a control tobacco plant when grown and cured under comparable conditions.

In one aspect, cured leaf from a modified tobacco plant produces or comprises less than 2, less than 1.8, less than 1.5, less than 1.2, less than 1.0, less than 0.8, less than 0.6, less than 0.4, less than 0.3, less than 0.2, less than 0.15, less than 0.1, or less than 0.05 ppm total TSNAs. In one aspect, cured leaf from a modified tobacco plant comprises between 2 and 0.05, between 1.8 and 0.05, between 1.5 and 0.05, between 1.2 and 0.05, between 1.0 and 0.05, between 0.8 and 0.05, between 0.6 and 0.05, between 0.4 and 0.05, between 0.3 and 0.05, between 0.2 and 0.05, between 0.15 and 0.05, or between 0.1 and 0.05 ppm total TSNAs. In one aspect, cured leaf from a modified tobacco plant comprises between 2 and 0.05, between 1.8 and 0.1, between 1.5 and 0.15, between 1.2 and 0.2, between 1.0 and 0.3, between 0.8 and 0.4, or between 0.6 and 0.5 ppm total TSNAs.

In one aspect, cured leaf from a modified tobacco plant comprises or produces less than 0.08 ppm NNK, wherein the level of the NNK level is measured based on a freeze-dried cured leaf sample using liquid chromatograph with tandem mass spectrometry (LC/MS/MS).

As used herein, “comparable conditions” refers to similar environmental conditions, agronomic practices, and/or curing process for growing or curing tobacco and making meaningful comparisons between two or more plant genotypes so that neither environmental conditions nor agronomic practices (including curing process) would contribute to, or explain, any differences observed between the two or more plant genotypes. Environmental conditions include, for example, light, temperature, water, humidity, and nutrition (e.g., nitrogen and phosphorus). Agronomic practices include, for example, seeding, clipping, undercutting, transplanting, topping, suckering, and curing. See Chapters 4B and 4C of Tobacco, Production, Chemistry and Technology, Davis & Nielsen, eds., Blackwell Publishing, Oxford (1999), pp. 70-103.

As used herein, a “reduced” or “increased” level refers to a statistically significant change (reduction or increase) from a reference point. As used herein, “statistically significant” refers to a p-value of less than 0.05, a p-value of less than 0.025, a p-value of less than 0.01, or a p-value of less than 0.001 when using an appropriate measure of statistical significance (e.g., a one-tailed two sample t-test).

As used herein, a “control plant” refers to a comparator plant that is an unmodified tobacco plant of the same variety or a tobacco plant having no transgene of interest, depending on the context or the purpose of the control plant. Control tobacco plants and plants of interest are grown and cured under comparable conditions.

In one aspect, a modified tobacco plant provided herein has similar or higher leaf yield compared to a control tobacco plant when grown and cured under comparable conditions. In an aspect, leaf yield is selected from the group consisting of fresh yield, dry yield, and cured yield. In one aspect, a modified tobacco plant provided herein produces a leaf yield mass within about 50%, within about 45%, within about 40%, within about 35%, within about 30%, within about 25%, within about 20%, within about 15%, within about 10%, within about 5%, within about 4%, within about 3%, within about 2%, within about 1%, or within about 0.5% compared to a control tobacco plant when grown and cured under comparable conditions. In another aspect, a modified tobacco plant provided herein produces a leaf yield mass at least 0.25%, 0.5%, 1%, 2.5%, 5%, 10%, 15%, 20%, 25%, 30%, 40%, 50%, 60%, 70%, 80%, 90%, 100%, or more than 100% higher compared to a control tobacco plant when grown and cured under comparable conditions. In another aspect, a modified tobacco plant provided herein produces a leaf yield mass 0.25%-100%, 0.5%-100%, 1%-100%, 2.5%-100%, 5%-100%, 10%-100%, 15%-100%, 20%-100%, 25%-100%, 30%-100%, 40%-100%, 50%-100%, 60%-100%, 70%-100%, 80%-100%, 90%-100%, 100%-200%, 100%-175%, 100%-150%, 100%-125%, 0.25%-50%, 0.5%-50%, 1%-50%, 2.5%-50%, 5%-50%, 10%-50%, 15%-50%, 20%-50%, 25%-50%, 30%-50%, 40%-50%, 50%-200%, 50%-175%, 50%-150%, 50%-125%, 0.25%-25%, 0.5%-25%, 1%-25%, 2.5%-25%, 5%-25%, 10%-25%, 15%-25%, 20%-25%, 25%-200%, 25%-175%, 25%-150%, or 25%-125% higher compared to a control tobacco plant when grown and cured under comparable conditions.

In one aspect, a modified tobacco plant provided herein has a similar or comparable plant height compared to a control tobacco plant when grown and cured under comparable conditions. In one aspect, a modified tobacco plant provided herein comprises a height within about 50%, within about 45%, within about 40%, within about 35%, within about 30%, within about 25%, within about 20%, within about 15%, within about 10%, within about 5%, within about 4%, within about 3%, within about 2%, within about 1%, or within about 0.5% compared to a control tobacco plants when grown and cured under comparable conditions. In another aspect, a modified tobacco plant provided herein comprises a height 0.25%, 0.5%, 1%, 2.5%, 5%, 10%, 15%, 20%, 25%, 30%, 40%, 50%, 60%, 70%, 80%, 90%, 100%, or more than 100% taller compared to a control tobacco plant when grown and cured under comparable conditions. In another aspect, a modified tobacco plant comprises a height 0.25%-100%, 0.5%-100%, 1%-100%, 2.5%-100%, 5%-100%, 10%-100%, 15%-100%, 20%-100%, 25%-100%, 30%-100%, 40%-100%, 50%-100%, 60%-100%, 70%-100%, 80%-100%, 90%-100%, 100%-200%, 100%-175%, 100%-150%, 100%-125%, 0.25%-50%, 0.5%-50%, 1%-50%, 2.5%-50%, 5%-50%, 10%-50%, 15%-50%, 20%-50%, 25%-50%, 30%-50%, 40%-50%, 50%-200%, 50%-175%, 50%-150%, 50%-125%, 0.25%-25%, 0.5%-25%, 1%-25%, 2.5%-25%, 5%-25%, 10%-25%, 15%-25%, 20%-25%, 25%-200%, 25%-175%, 25%-150%, or 25%-125% taller compared to a control tobacco plant when grown and cured under comparable conditions.

In one aspect, a modified tobacco plant provided herein produces leaf that has a similar or higher USDA grade index value compared to a control tobacco plant when grown and cured under comparable conditions. In one aspect, a modified tobacco plant provided herein produces leaf with a USDA grade index value within about 50%, within about 45%, within about 40%, within about 35%, within about 30%, within about 25%, within about 20%, within about 15%, within about 10%, within about 5%, within about 4%, within about 3%, within about 2%, within about 1%, or within about 0.5% compared to a control tobacco plant when grown and cured under comparable conditions. In one aspect, a modified tobacco plant provided herein is capable of producing leaf having a USDA grade index value of 55 or more, 60 or more, 65 or more, 70 or more, 75 or more, 80 or more, 85 or more, 90 or more, or 95 or more. In one aspect, a modified tobacco plant provided herein produces leaf with a USDA grade index value at least 1, 2, 3, 4, 5, 6, 7, 8, 9, 10, 12, 13, 14, 15, 16, 17, 18, 19, 20, 21, 22, 23, 24, 25, 26, 27, 28, 29, 30, 35, 40, 45, 50, or more than 50 units higher compared to a control tobacco plant when grown and cured under comparable conditions. In one aspect, a modified tobacco plant provided herein produces leaf with a USDA grade index value 1-50, 1-45, 1-40, 1-35, 1-30, 1-29, 1-28, 1-27, 1-26, 1-25, 1-24, 1-23, 1-22, 1-21, 1-20, 1-19, 1-18, 1-17, 1-16, 1-15, 1-14, 1-13, 1-12, 1-11, 1-10, 1-9, 1-8, 1-7, 1-6, 1-5, 1-4, 1-3, 1-2, 2-50, 2-45, 2-40, 2-35, 2-30, 2-29, 2-28, 2-27, 2-26, 2-25, 2-24, 2-23, 2-22, 2-21, 2-20, 2-19, 2-18, 2-17, 2-16, 2-15, 2-14, 2-13, 2-12, 2-11, 2-10, 2-9, 2-8, 2-7, 2-6, 2-5, 2-4, 2-3, 3-50, 3-45, 3-40, 3-35, 3-30, 3-29, 3-28, 3-27, 3-26, 3-25, 3-24, 3-23, 3-22, 3-21, 3-20, 3-19, 3-18, 3-17, 3-16, 3-15, 3-14, 3-13, 3-12, 3-11, 3-10, 3-9, 3-8, 3-7, 3-6, 3-5, 3-4, 4-50, 4-45, 4-40, 4-35, 4-30, 4-29, 4-28, 4-27, 4-26, 4-25, 4-24, 4-23, 4-22, 4-21, 4-20, 4-19, 4-18, 4-17, 4-16, 4-15, 4-14, 4-13, 4-12, 4-11, 4-10, 4-9, 4-8, 4-7, 4-6, 4-5, 5-50, 5-45, 5-40, 5-35, 5-30, 5-29, 5-28, 5-27, 5-26, 5-25, 5-24, 5-23, 5-22, 5-21, 5-20, 5-19, 5-18, 5-17, 5-16, 5-15, 5-14, 5-13, 5-12, 5-11, 5-10, 5-9, 5-8, 5-7, 5-6, 10-50, 10-40, 10-30, 10-20, 20-50, 20-30, 20-40, or 20-30 units higher compared to a control tobacco plant when grown and cured under comparable conditions.

Tobacco grades are evaluated based on factors including, but not limited to, the leaf stalk position, leaf size, leaf color, leaf uniformity and integrity, ripeness, texture, elasticity, sheen (related with the intensity and the depth of coloration of the leaf as well as the shine), hygroscopicity (the faculty of the tobacco leaf to absorb and to retain the ambient moisture), and green nuance or cast. Leaf grade can be determined, for example, using an Official Standard Grade published by the Agricultural Marketing Service of the US Department of Agriculture (7 U.S.C. § 511). See, e.g., Official Standard Grades for Burley Tobacco (U.S. Type 31 and Foreign Type 93), effective Nov. 5, 1990 (55 F.R. 40645); Official Standard Grades for Flue-Cured Tobacco (U.S. Types 11, 12, 13, 14 and Foreign Type 92), effective Mar. 27, 1989 (54 F.R. 7925); Official Standard Grades for Pennsylvania Seedleaf Tobacco (U.S. Type 41), effective Jan. 8, 1965 (29 F.R. 16854); Official Standard Grades for Ohio Cigar-Leaf Tobacco (U.S. Types 42, 43, and 44), effective Dec. 8, 1963 (28 F.R. 11719 and 28 F.R. 11926); Official Standard Grades for Wisconsin Cigar-Binder Tobacco (U.S. Types 54 and 55), effective Nov. 20, 1969 (34 F.R. 17061); Official Standard Grades for Wisconsin Cigar-Binder Tobacco (U.S. Types 54 and 55), effective Nov. 20, 1969 (34 F.R. 17061); Official Standard Grades for Georgia and Florida Shade-Grown Cigar-Wrapper Tobacco (U.S. Type 62), Effective April 1971. A USDA grade index value can be determined according to an industry accepted grade index. See, e.g., Bowman et al, Tobacco Science, 32:39-40 (1988); Legacy Tobacco Document Library (Bates Document #523267826-523267833, Jul. 1, 1988, Memorandum on the Proposed Burley Tobacco Grade Index); and Miller et al., 1990, Tobacco Intern., 192:55-57 (all foregoing references are incorporated by inference in their entirety). Alternatively, leaf grade can be determined via hyper-spectral imaging. See e.g., WO 2011/027315 (published on Mar. 10, 2011, and incorporated by inference in its entirety).

In one aspect, a modified tobacco plant provided herein comprises tobacco leaf with reduced total TSNAs and further comprises one or more desirable or enhanced properties, e.g., inhibited or reduced sucker growth prior to or after topping. In one aspect, a modified plant provided herein comprises fewer total suckers, smaller suckers, or both compared to a control plant lacking such modification when grown and cured under comparable conditions. In one aspect, smaller suckers of a modified plant provided herein comprise reduced mass, reduced length, reduced diameter, or a combination thereof compared to suckers of a control plant grown and cured under comparable conditions.

Unless specified otherwise, measurements of the level of total TSNAs, individual TSNA, total or individual alkaloid, total or individual antioxidant, leaf yield, or leaf grade index values mentioned herein for cured leaf from a tobacco plant, variety, cultivar, or line refer to average measurements, including, for example, an average of multiple leaves (e.g., 2, 3, 4, 5, 6, 7, 8, 9, 10, 11, 12, 13, 14, 15, 16, 17, 18, 19, or 20 or more leaves) of a single plant or an average measurement from a population of tobacco plants from a single variety, cultivar, or line. A population of tobacco plants or a collection of tobacco leaf for determining an average measurement (e.g., fresh weight or leaf grading) can be of any size, for example, 5, 10, 15, 20, 25, 30, 35, 40, or 50. Industry-accepted standard protocols are followed for determining average measurements or grade index values.

In one aspect, a modified tobacco plant or leaf provided here has a similar leaf chemistry profile compared to a control plant when grown and cured under comparable conditions. Without being limiting, a leaf chemistry profile can comprise the amount of alkaloids (e.g., nicotine, nornicotine, anabasine, anatabine), malic acid, and reducing sugars (e.g., dextrose), or a combination thereof in a tobacco plant or tobacco leaf. In one aspect, a modified plant or leaf provided herein comprises a total alkaloids level within about 90%, within about 80%, within about 70%, within about 60%, within about 50%, within about 45%, within about 40%, within about 35%, within about 30%, within about 25%, within about 20%, within about 15%, within about 10%, within about 5%, within about 4%, within about 3%, within about 2%, within about 1%, or within about 0.5% of the total alkaloids level of a control plant when grown and cured under comparable conditions. In one aspect, a modified plant or leaf provided herein comprises a nicotine level within about 90%, within about 80%, within about 70%, within about 60%, within about 50%, within about 45%, within about 40%, within about 35%, within about 30%, within about 25%, within about 20%, within about 15%, within about 10%, within about 5%, within about 4%, within about 3%, within about 2%, within about 1%, or within about 0.5% of the nicotine level of a control plant when grown and cured under comparable conditions. In one aspect, a modified plant or leaf provided herein comprises a nornicotine level within about 50%, within about 45%, within about 40%, within about 35%, within about 30%, within about 25%, within about 20%, within about 15%, within about 10%, within about 5%, within about 4%, within about 3%, within about 2%, within about 1%, or within about 0.5% of the nornicotine level of a control plant when grown and cured under comparable conditions. In one aspect, a modified plant or leaf provided herein comprises an anabasine level within about 50%, within about 45%, within about 40%, within about 35%, within about 30%, within about 25%, within about 20%, within about 15%, within about 10%, within about 5%, within about 4%, within about 3%, within about 2%, within about 1%, or within about 0.5% of the anabasine level of a control plant when grown and cured under comparable conditions. In one aspect, a modified plant or leaf provided herein comprises an anatabine level within about 50%, within about 45%, within about 40%, within about 35%, within about 30%, within about 25%, within about 20%, within about 15%, within about 10%, within about 5%, within about 4%, within about 3%, within about 2%, within about 1%, or within about 0.5% of the anatabine level of a control plant when grown and cured under comparable conditions. In one aspect, a modified plant or leaf provided herein comprises a malic acid level within about 50%, within about 45%, within about 40%, within about 35%, within about 30%, within about 25%, within about 20%, within about 15%, within about 10%, within about 5%, within about 4%, within about 3%, within about 2%, within about 1%, or within about 0.5% of the malic acid level of a control plant when grown and cured under comparable conditions. In one aspect, a modified plant or leaf provided herein comprises a reducing sugars level within about 50%, within about 45%, within about 40%, within about 35%, within about 30%, within about 25%, within about 20%, within about 15%, within about 10%, within about 5%, within about 4%, within about 3%, within about 2%, within about 1%, or within about 0.5% of the reducing sugars level of a control plant when grown and cured under comparable conditions. In one aspect, a modified plant or leaf provided herein comprises a dextrose level within about 50%, within about 45%, within about 40%, within about 35%, within about 30%, within about 25%, within about 20%, within about 15%, within about 10%, within about 5%, within about 4%, within about 3%, within about 2%, within about 1%, or within about 0.5% of the dextrose level of a control plant when grown and cured under comparable conditions.

In one aspect, a plant component provided herein includes, but is not limited to, a leaf, a stem, a root, a seed, a flower, pollen, an anther, an ovule, a pedicel, a fruit, a meristem, a cotyledon, a hypocotyl, a pod, an embryo, endosperm, an explant, a callus, a tissue culture, a shoot, a cell, and a protoplast. In further aspects, this disclosure provides tobacco plant cells, tissues, and organs that are not reproductive material and do not mediate the natural reproduction of the plant. In another aspect, this disclosure also provides tobacco plant cells, tissues, and organs that are reproductive material and mediate the natural reproduction of the plant. In another aspect, this disclosure provides tobacco plant cells, tissues, and organs that cannot maintain themselves via photosynthesis. In another aspect, this disclosure provides somatic tobacco plant cells. Somatic cells, contrary to germline cells, do not mediate plant reproduction.

Provided cells, tissues and organs can be from seed, fruit, leaf, cotyledon, hypocotyl, meristem, embryos, endosperm, root, shoot, stem, pod, flower, inflorescence, stalk, pedicel, style, stigma, receptacle, petal, sepal, pollen, anther, filament, ovary, ovule, pericarp, phloem, and vascular tissue. In another aspect, this disclosure provides a tobacco plant chloroplast. In a further aspect, this disclosure provides an epidermal cell, a stomata cell, a leaf hair (Trichome), a root hair, or a storage root. In another aspect, this disclosure provides a tobacco protoplast.

Skilled artisans understand that tobacco plants naturally reproduce via seeds, not via asexual reproduction or vegetative propagation. In one aspect, this disclosure provides tobacco endosperm. In another aspect, this disclosure provides a tobacco endosperm cell. In a further aspect, this disclosure provides a male or female sterile tobacco plant, which cannot reproduce without human intervention.

In one aspect, a modified plant, seed, plant part, or plant cell provided herein comprises one or more non-naturally occurring mutations. In one aspect, a mutation provided herein suppresses TSNA levels in cured leaf from a tobacco plant. Types of mutations provided herein include, for example, substitutions (point mutations), deletions, insertions, duplications, and inversions. Such mutations are desirably present in the coding region of a gene; however, mutations in a promoter or other regulatory region, an intron, an intron-exon boundary, or an untranslated region of a gene may also be desirable.

In one aspect, a modified tobacco plant comprises one or more mutations or modifications capable of providing the reduced level of one or more TSNAs. In another aspect, one or more mutations are further capable of providing one or more traits selected from the group consisting of: i. a reduced level of nitrite, ii. an increased level of oxygen radicle absorbance capacity (ORAC), and iii. an increased level of one or more antioxidants; wherein the reduced or increased level is compared to a control tobacco plant or cured leaf from a control tobacco plant when grown and cured under comparable. In one aspect, a mutation comprises a mutation type selected from the group consisting of an insertion, a deletion, an inversion, a duplication, a substitution, and a combination thereof.

In one aspect, a modified tobacco plant comprises one or more mutations or modifications capable of activating one or more genes encoding a biosynthetic enzyme, a regulatory transcription factor, a transporter, a catabolic enzyme, or a combination thereof, for one or more antioxidants. In another aspect, one or more mutations or modifications are in one or more genes encoding a biosynthetic enzyme, a regulatory transcription factor, a transporter, a catabolic enzyme, or a combination thereof, for one or more antioxidants selected from the group consisting of anthocyanidin, flavanone, flavanol, flavone, flavonol, isoflavone, hydroxybenzoic acid, hydroxycinnamic acid, ellagitannin, stibene, lignan, carotenoids, and glycyrrhzin. In a further aspect, one or more mutations or modifications are in one or more genes encoding a biosynthetic enzyme, a regulatory transcription factor, a transporter, a catabolic enzyme, or a combination thereof, for one or more antioxidants selected from the group consisting of Delphnidin, Cyanidin, Procyanidin, Prodelphinidin, Hesperetin, Naringenin, Catechin, Epicatechin, Apigenin, Luteonin, Quercetin, Myricetin, Rutin, Genistein, Daidzein, Gallic acid, Vanillic acid, Protocatechuic acid, Ferunic acid, Cinnamic acid, Coumeric acid, Chlorogenic acid, Coffeic acid, ferulic acid, Sanguiin, Resveratrol, Sesamin, Caretonoids, and Vitamin C.

In one aspect, a modified tobacco plant of the present specification comprises tobacco leaves with increased levels of anthocyanins. In a further aspect, a modified tobacco plant with increased levels of anthocyanins further comprises leaves that have a purple or crimson visual appearance. In one aspect, a modified tobacco plant of the present specification comprises tobacco leaves with increased levels of antioxidants and without increased levels of anthocyanins. In a further aspect, a modified tobacco plant comprising increased levels of antioxidants and without increased levels of anthocyanins further comprises leaves with a visual appearance similar to an unmodified tobacco plant.

As used herein, a “biosynthetic enzyme” refers to a protein that functions in the synthesis of antioxidants, alkaloids, TSNAs, nitrite, nitrate, Chlorogenic Acid or other proteins affecting the activity or stability of antioxidants, alkaloids, TSNAs, nitrite, nitrate or Chlorogenic Acid. These proteins catalyze reactions that result in the transformation of one molecular structure into another structure as part of a biosynthesis pathway. Exemplary biosynthetic enzymes include but are limited to Anthocyanidin synthase2 (NtANS2), Dihyfroflavonol-4-reductase (NtDFR2), Shikimate O-hydroxycinnamoyl transferase (HCT) and Hydroxycinnamoyl CoA quinate Transferase (HQT). The activity of a biosynthetic enzyme effects the total concentration of different molecule species that compose a biosynthetic pathway.

As used herein, a “regulatory transcription factor” is a protein that binds a promoter element of a target gene to modulate the transcription of one or more genes involved in antioxidant biosynthesis, transport, catabolism, or other processes affecting the level of one or more antioxidants. Exemplary regulatory transcription factors include AtPAP1, NtPAP1, NtMYB3-like, NtJAF13, and AtTTG1. A regulatory transcription factor can bind DNA as part of a protein complex or individually. A regulatory transcription factor can have a single target or multiple targets and can bind different targets with varying affinities. The activity of a regulatory transcription factor can be to activate, repress, or attenuate transcription from a target loci.

As used herein, a “transport protein” can be a transmembrane protein that actively or passively moves molecules across a biological membrane. A transport protein can aid in the movement of ions, small molecules or macromolecules. A transport protein can be referred to as a transmembrane transporter, a transmembrane pump, an anion transport protein, a cation transport protein, or an escort protein. Transport proteins can also facilitate the movement of molecules or proteins in vesicles composed of biological membrane. A transport protein can be integrated into a biological membrane. A Transport protein can be anchored to a biological membrane via different modifications such as but not limited to myristolation, prenylation or palmitoylation.

In one aspect, a modified tobacco plant comprises one or more mutations in a gene encoding a polypeptide having at least 50%, 55%, 60%, 65%, 70%, 75%, 80%, 85%, 90%, 95%, 97%, 98%, 99%, or 100% identity to a sequence selected from the group consisting of SEQ ID No. 1 to 23 and 47 to 52. In another aspect, a modified tobacco plant comprises one or more mutations in a gene comprise a coding sequence having at least 50%, 55%, 60%, 65%, 70%, 75%, 80%, 85%, 90%, 95%, 97%, 98%, 99%, or 100% identity to a sequence selected from the group consisting of SEQ ID No. 24 to 46 and 53 to 58.

In one aspect, a modified plant, seed, plant component, plant cell, or plant genome provided herein comprises one or more transgenes. As used herein, a “transgene” refers to a polynucleotide that has been transferred into a genome by any method known in the art. In one aspect, a transgene is an exogenous polynucleotide. In one aspect, a transgene is an endogenous polynucleotide that is integrated into a new genomic locus where it is not normally found.

As used herein, “modified”, in the context of plants, seeds, plant components, plant cells, and plant genomes, refers to a state containing changes or variations from their natural or native state. For instance, a “native transcript” of a gene refers to an RNA transcript that is generated from an unmodified gene. Typically, a native transcript is a sense transcript. Modified plants or seeds contain molecular changes in their genetic materials, including either genetic or epigenetic modifications. Typically, modified plants or seeds, or a parental or progenitor line thereof, have been subjected to mutagenesis, genome editing (e.g., without being limiting, via methods using site-specific nucleases), genetic transformation (e.g., without being limiting, via methods of Agrobacterium transformation or microprojectile bombardment), or a combination thereof. In one aspect, a modified plant provided herein comprises no non-plant genetic material or sequences. In yet another aspect, a modified plant provided herein comprises no interspecies genetic material or sequences. In one aspect, this disclosure provides methods and compositions related to modified plants, seeds, plant components, plant cells, and products made from modified plants, seeds, plant parts, and plant cells. In one aspect, a modified seed provided herein gives rise to a modified plant provided herein. In one aspect, a modified plant, seed, plant component, plant cell, or plant genome provided herein comprises a recombinant DNA construct or vector provided herein. In another aspect, a product provided herein comprises a modified plant, plant component, plant cell, or plant chromosome or genome provided herein. The present disclosure provides modified plants with desirable or enhanced properties, e.g., without being limiting, disease, insect, or pest tolerance (for example, virus tolerance, bacteria tolerance, fungus tolerance, nematode tolerance, arthropod tolerance, gastropod tolerance); herbicide tolerance; environmental stress resistance; quality improvements such as yield, nutritional enhancements, environmental or stress tolerances; any desirable changes in plant physiology, growth, development, morphology or plant product(s) including starch production, modified oils production, high oil production, modified fatty acid content, high protein production, fruit ripening, enhanced animal and human nutrition, biopolymer production, pharmaceutical peptides and secretable peptides production; improved processing traits; improved digestibility; low raffinose; industrial enzyme production; improved flavor; nitrogen fixation; hybrid seed production; and fiber production.

As used herein, “genome editing” or editing refers to targeted mutagenesis, insertion, deletion, inversion, substitution, or translocation of a nucleotide sequence of interest in a genome using a targeted editing technique. A nucleotide sequence of interest can be of any length, e.g., at least 1, at least 2, at least 3, at least 4, at least 5, at least 6, at least 7, at least 8, at least 9, at least 10, at least 15, at least 20, at least 25, at least 30, at least 35, at least 40, at least 45, at least 50, at least 75, at least 100, at least 250, at least 500, at least 1000, at least 2500, at least 5000, at least 10,000, or at least 25,000 nucleotides. As used herein, a “targeted editing technique” refers to any method, protocol, or technique that allows the precise and/or targeted editing of a specific location in a genome (e.g., the editing is not random). Without being limiting, use of a site-specific nuclease is one example of a targeted editing technique. Another non-limiting example of a targeted editing technique is the use of one or more tether guide Oligos (tgOligos). As used herein, a “targeted edit” refers to a targeted mutagenesis, insertion, deletion, inversion, or substitution caused by a targeted editing technique. A nucleotide sequence of interest can be an endogenous genomic sequence or a transgenic sequence.

In one aspect, a “targeted editing technique” refers to any method, protocol, or technique that allows the precise and/or targeted editing of a specific location in a genome (e.g., the editing is not random). Without being limiting, use of a site-specific nuclease is one example of a targeted editing technique.

In one aspect, a targeted editing technique is used to edit an endogenous locus or an endogenous gene. In another aspect, a targeted editing technique is used to edit a transgene. As used herein, an “endogenous gene” or a “native copy” of a gene refers to a gene that originates from within a given organism, cell, tissue, genome, or chromosome. An “endogenous gene” or a “native copy” of a gene is a gene that was not previously modified by human action.

In one aspect, a modified tobacco plant described here comprises one or more mutations are introduced via a system selected from the group consisting of chemical mutagenesis, irradiation mutagenesis, transposon mutagenesis, Agrobacterium-mediated transformation, a meganuclease, a zinc-finger nuclease (ZFN), a transcription activator-like effector nuclease (TALEN), a clustered regularly-interspaced short palindromic repeats (CRISPR)/Cas9 system, a CRISPR/Cpf1 system, a CRISPR/Csm1 system, and a combination thereof (see, for example, U.S. Patent Application publication 2017/0233756).

In one aspect, methods provided herein are capable of producing a tobacco plant comprising a reduced level of one or more TSNAs using mutagenesis. Mutagenesis methods include, without limitation, chemical mutagenesis, for example, treatment of seeds with ethyl methylsulfate (EMS) (Hildering and Verkerk, In, The use of induced mutations in plant breeding. Pergamon Press, pp. 317-320, 1965); or UV-irradiation, X-rays, electron beams, ion beams (e.g., carbon ion beam, helium ion beam, neon ion beam), and fast neutron irradiation (see, for example, Verkerk, Neth. J. Agric. Sci. 19:197-203, 1971; Pochlman, Breeding Field Crops, Van Nostrand Reinhold, New York (3.sup.rd ed.), 1987; and Tanaka, J. Radiat. Res. 51:223-233, 2010); transposon tagging (Fedoroff et al., 1984; U.S. Pat. Nos. 4,732,856 and 5,013,658); and T-DNA insertion methodologies (Hockema et al., 1983; U.S. Pat. No. 5,149,645). EMS-induced mutagenesis consists of chemically inducing random point mutations over the length of a genome. Fast neutron mutagenesis consists of exposing seeds to neutron bombardment which causes large deletions through double stranded DNA breakage. Transposon tagging comprises inserting a transposon within an endogenous gene to reduce or eliminate expression of the gene.

In addition, a fast and automatable method for screening for chemically induced mutations, TILLING (Targeting Induced Local Lesions In Genomes), using denaturing HPLC or selective endonuclease digestion of selected PCR products is also applicable to the present disclosure. See, McCallum et al. (2000) Nat. Biotechnol. 18:455-457. Mutations that impact gene expression or that interfere with the function of genes provided herein can be determined using methods that are well known in the art. Insertional mutations in gene exons usually result in null-mutants. Mutations in conserved residues can be particularly effective in inhibiting the function of a protein.

The screening and selection of mutagenized tobacco plants can be through any methodologies known to those having ordinary skill in the art. Examples of screening and selection methodologies include, but are not limited to, Southern analysis, PCR amplification for detection of a polynucleotide, Northern blots, RNase protection, primer-extension, RT-PCR amplification for detecting RNA transcripts, Sanger sequencing, Next Generation sequencing technologies (e.g., Illumina, PacBio, Ion Torrent, 454) enzymatic assays for detecting enzyme or ribozyme activity of polypeptides and polynucleotides, and protein gel electrophoresis, Western blots, immunoprecipitation, and enzyme-linked immunoassays to detect polypeptides. Other techniques such as in situ hybridization, enzyme staining, and immunostaining also can be used to detect the presence or expression of polypeptides and/or polynucleotides. Methods for performing all of the referenced techniques are known.

In one aspect, a modified plant or plant genome provided herein is mutated or edited by a nuclease selected from the group consisting of a meganuclease, a zinc-finger nuclease (ZFN), a transcription activator-like effector nuclease (TALEN), a CRISPR/Cas9 nuclease, a CRISPR/Cpf1 nuclease, or a CRISPR/Csm1 nuclease. As used herein, “editing” or “genome editing” refers to targeted mutagenesis of at least 1, at least 2, at least 3, at least 4, at least 5, at least 6, at least 7, at least 8, at least 9, or at least 10 nucleotides of an endogenous plant genome nucleic acid sequence, or removal or replacement of an endogenous plant genome nucleic acid sequence. In one aspect, an edited nucleic acid sequence provided herein has at least 99.9%, at least 99.5%, at least 99%, at least 98%, at least 97%, at least 96%, at least 95%, at least 94%, at least 93%, at least 92%, at least 91%, at least 90%, at least 85%, at least 80%, or at least 75% sequence identity with an endogenous nucleic acid sequence. In one aspect, an edited nucleic acid sequence provided herein has at least 99.9%, at least 99.5%, at least 99%, at least 98%, at least 97%, at least 96%, at least 95%, at least 94%, at least 93%, at least 92%, at least 91%, at least 90%, at least 85%, at least 80%, or at least 75% sequence identity with a polynucleotide selected from the group consisting of SEQ ID NOs: 24 to 46 and 53 to 58, and fragments thereof. In another aspect, an edited nucleic acid sequence provided herein has at least 99.9%, at least 99.5%, at least 99%, at least 98%, at least 97%, at least 96%, at least 95%, at least 94%, at least 93%, at least 92%, at least 91%, at least 90%, at least 85%, at least 80%, or at least 75% sequence identity with a polynucleotide encoding a polypeptide selected from the group consisting of SEQ ID NOs: 1 to 23 and 47 to 52.

Meganucleases, ZFNs, TALENs, CRISPR/Cas9, CRISPR/Csm1, and CRISPR/Cpf1 induce a double-strand DNA break at a target site of a genomic sequence that is then repaired by the natural processes of homologous recombination (HR) or non-homologous end-joining (NHEJ). Sequence modifications then occur at the cleaved sites, which can include deletions or insertions that result in gene disruption in the case of NHEJ, or integration of donor nucleic acid sequences by HR. In one aspect, a method provided herein comprises editing a plant genome with a nuclease provided herein to mutate at least 1, at least 2, at least 3, at least 4, at least 5, at least 6, at least 7, at least 8, at least 9, at least 10, or more than 10 nucleotides in the plant genome via HR with a donor polynucleotide. In one aspect, a mutation provided herein is caused by genome editing using a nuclease. In another aspect, a mutation provided herein is caused by non-homologous end-joining or homologous recombination.

In one aspect, a mutation provided herein provides a dominant mutant that activates the expression or activity of a gene of interest, e.g., a gene selected from the group consisting of a biosynthetic enzyme, a regulatory transcription factor, a transporter, a catabolic enzyme, or a combination thereof, for one or more antioxidants.

Meganucleases, which are commonly identified in microbes, are unique enzymes with high activity and long recognition sequences (>14 bp) resulting in site-specific digestion of target DNA. Engineered versions of naturally occurring meganucleases typically have extended DNA recognition sequences (for example, 14 to 40 bp). The engineering of meganucleases can be more challenging than that of ZFNs and TALENs because the DNA recognition and cleavage functions of meganucleases are intertwined in a single domain. Specialized methods of mutagenesis and high-throughput screening have been used to create novel meganuclease variants that recognize unique sequences and possess improved nuclease activity.

ZFNs are synthetic proteins consisting of an engineered zinc finger DNA-binding domain fused to the cleavage domain of the FokI restriction endonuclease. ZFNs can be designed to cleave almost any long stretch of double-stranded DNA for modification of the zinc finger DNA-binding domain. ZFNs form dimers from monomers composed of a non-specific DNA cleavage domain of FokI endonuclease fused to a zinc finger array engineered to bind a target DNA sequence.

The DNA-binding domain of a ZFN is typically composed of 3-4 zinc-finger arrays. The amino acids at positions −1, +2, +3, and +6 relative to the start of the zinc finger co-helix, which contribute to site-specific binding to the target DNA, can be changed and customized to fit specific target sequences. The other amino acids form the consensus backbone to generate ZFNs with different sequence specificities. Rules for selecting target sequences for ZFNs are known in the art.

The FokI nuclease domain requires dimerization to cleave DNA and therefore two ZFNs with their C-terminal regions are needed to bind opposite DNA strands of the cleavage site (separated by 5-7 bp). The ZFN monomer can cute the target site if the two-ZF-binding sites are palindromic. The term ZFN, as used herein, is broad and includes a monomeric ZFN that can cleave double stranded DNA without assistance from another ZFN. The term ZFN is also used to refer to one or both members of a pair of ZFNs that are engineered to work together to cleave DNA at the same site.

Without being limited by any scientific theory, because the DNA-binding specificities of zinc finger domains can in principle be re-engineered using one of various methods, customized ZFNs can theoretically be constructed to target nearly any gene sequence. Publicly available methods for engineering zinc finger domains include Context-dependent Assembly (CoDA), Oligomerized Pool Engineering (OPEN), and Modular Assembly.

TALENs are artificial restriction enzymes generated by fusing the transcription activator-like effector (TALE) DNA binding domain to a FokI nuclease domain. When each member of a TALEN pair binds to the DNA sites flanking a target site, the FokI monomers dimerize and cause a double-stranded DNA break at the target site. The term TALEN, as used herein, is broad and includes a monomeric TALEN that can cleave double stranded DNA without assistance from another TALEN. The term TALEN is also used to refer to one or both members of a pair of TALENs that work together to cleave DNA at the same site.

Transcription activator-like effectors (TALEs) can be engineered to bind practically any DNA sequence. TALE proteins are DNA-binding domains derived from various plant bacterial pathogens of the genus Xanthomonas. The X pathogens secrete TALEs into the host plant cell during infection. The TALE moves to the nucleus, where it recognizes and binds to a specific DNA sequence in the promoter region of a specific DNA sequence in the promoter region of a specific gene in the host genome. TALE has a central DNA-binding domain composed of 13-28 repeat monomers of 33-34 amino acids. The amino acids of each monomer are highly conserved, except for hypervariable amino acid residues at positions 12 and 13. The two variable amino acids are called repeat-variable diresidues (RVDs). The amino acid pairs NI, NG, HD, and NN of RVDs preferentially recognize adenine, thymine, cytosine, and guanine/adenine, respectively, and modulation of RVDs can recognize consecutive DNA bases. This simple relationship between amino acid sequence and DNA recognition has allowed for the engineering of specific DNA binding domains by selecting a combination of repeat segments containing the appropriate RVDs.

Besides the wild-type FokI cleavage domain, variants of the FokI cleavage domain with mutations have been designed to improve cleavage specificity and cleavage activity. The FokI domain functions as a dimer, requiring two constructs with unique DNA binding domains for sites in the target genome with proper orientation and spacing. Both the number of amino acid residues between the TALEN DNA binding domain and the FokI cleavage domain and the number of bases between the two individual TALEN binding sites are parameters for achieving high levels of activity.

The relationship between amino acid sequence and DNA recognition of the TALE binding domain allows for designable proteins. Software programs such as DNA Works can be used to design TALE constructs. Other methods of designing TALE constructs are known to those of skill in the art. See Doyle et al., Nucleic Acids Research (2012) 40:W117-122.; Cermak et al., Nucleic Acids Research (2011). 39:e82; and tale-nt.cac.cornell.edu/about.

A CRISPR/Cas9 system, CRISPR/Csm1, or a CRISPR/Cpf1 system are alternatives to the FokI-based methods ZFN and TALEN. The CRISPR systems are based on RNA-guided engineered nucleases that use complementary base pairing to recognize DNA sequences at target sites.

CRISPR/Cas9, CRISPR/Csm1, and a CRISPR/Cpf1 systems are part of the adaptive immune system of bacteria and archaea, protecting them against invading nucleic acids such as viruses by cleaving the foreign DNA in a sequence-dependent manner. The immunity is acquired by the integration of short fragments of the invading DNA known as spacers between two adjacent repeats at the proximal end of a CRISPR locus. The CRISPR arrays, including the spacers, are transcribed during subsequent encounters with invasive DNA and are processed into small interfering CRISPR RNAs (crRNAs) approximately 40 nt in length, which combine with the trans-activating CRISPR RNA (tracrRNA) to activate and guide the Cas9 nuclease. This cleaves homologous double-stranded DNA sequences known as protospacers in the invading DNA. A prerequisite for cleavage is the presence of a conserved protospacer-adjacent motif (PAM) downstream of the target DNA, which usually has the sequence 5-NGG-3 but less frequently NAG. Specificity is provided by the so-called “seed sequence” approximately 12 bases upstream of the PAM, which must match between the RNA and target DNA. Cpf1 acts in a similar manner to Cas9, but Cpf1 does not require a tracrRNA.

In still another aspect, a modified tobacco plant provided herein further comprises one or more mutations in one or more loci encoding a nicotine demethylase (e.g., CYP82E4, CYP82E5, CYP82E10) that confer reduced amounts of nornicotine (See U.S. Pat. Nos. 8,319,011; 8,124,851; 9,187,759; 9,228,194; 9,228,195; 9,247,706) compared to control plant lacking one or more mutations in one or more loci encoding a nicotine demethylase. In one aspect, a modified tobacco plant described herein further comprises reduced nicotine demethylase activity compared to a control plant when grown and cured under comparable conditions. In another aspect, a modified tobacco plant described herein further comprises a reduced level of total alkaloids compared to the control plant when grown and cured under comparable conditions. In another aspect, a tobacco plant provided herein further comprises one or more mutations in a Nic1 locus, a Nic2 locus, or both, which confer reduced amounts of nicotine compared to a control plant lacking one or more mutations in a Nic1 locus, a Nic2 locus, or both. In another aspect, a modified tobacco plant described herein further comprises a reduced level of nicotine compared to the control plant when grown and cured under comparable conditions. In a further aspect, a modified tobacco plant described herein comprises a substantially similar level of nicotine compared to the control plant when grown and cured under comparable conditions.

In one aspect, a modified tobacco plant described herein is a cisgenic plant. As used herein, “cisgenesis” or “cisgenic” refers to genetic modification of a plant, plant cell, or plant genome in which all components (e.g., promoter, donor nucleic acid, selection gene) have only plant origins (i.e., no non-plant origin components are used). In one aspect, a modified plant, plant cell, or plant genome provided herein is cisgenic. Cisgenic plants, plant cells, and plant genomes provided herein can lead to ready-to-use tobacco lines. In another aspect, a modified tobacco plant provided herein comprises no non-tobacco genetic material or sequences. In one aspect, a cisgenic construct of the present specification encodes a polynucleotide selected from the group consisting of Ubi4-P:PAP1-HSP-T (SEQ ID NO:59), Ubi4-P:NtAN2-HSP-T (SEQ ID NO:60), Tub-P:NtAN2-HSP-T (SEQ ID NO:61), Ubi4-P:NtAN2-HSP-T:Tub-P:NtAN2-HSP-T (SEQ ID NO:62), and Ubi4-P:NtAN1a-HSP-T:Tub-P:NtAN2-HSP-T (SEQ ID NO:63).

In one aspect, a modified tobacco plant described herein comprises one or more transgenes or recombinant DNA constructs capable of providing a reduced level of one or more TSNAs compared to a control plant without the one or more transgenes. In another aspect, a modified tobacco plant comprises one or more transgenes or recombinant DNA constructs further providing the one or more traits selected from the group consisting of: i. a reduced level of nitrite, ii. an increased level of oxygen radicle absorbance capacity (ORAC), and iii. an increased level of one or more antioxidants; wherein the reduced or increased level is compared to a control tobacco plant when grown and cured under comparable.

In another aspect, a modified tobacco plant comprises one or more transgenes or recombinant DNA constructs encoding a biosynthetic enzyme, a regulatory transcription factor, a transporter, a catabolic enzyme, or a combination thereof, for one or more antioxidants selected from the group consisting of anthocyanidin, flavanone, flavanol, flavone, flavonol, isoflavone, hydroxybenzoic acid, hydroxycinnamic acid, ellagitannin, stibene, lignan, carotenoids, and glycyrrhzin. In another aspect, a modified tobacco plant comprises one or more transgenes or recombinant DNA constructs encoding a biosynthetic enzyme, a regulatory transcription factor, a transporter, a catabolic enzyme, or a combination thereof, for one or more antioxidants selected from the group consisting of Delphnidin, Cyanidin, Procyanidin, Prodelphinidin, Hesperetin, Naringenin, Catechin, Epicatechin, Apigenin, Luteonin, Quercetin, Myricetin, Rutin, Genistein, Daidzein, Gallic acid, Vanillic acid, Protocatechuic acid, Ferunic acid, Cinnamic acid, Coumeric acid, Chlorogenic acid, Coffeic acid, ferulic acid, Sanguiin, Resveratrol, Sesamin, Caretonoids, and Vitamin C. In one aspect, one or more transgenes or recombinant DNA constructs encode a polypeptide having at least 50%, 55%, 60%, 65%, 70%, 75%, 80%, 85%, 90%, 95%, 97%, 98%, 99%, or 100% identity to a sequence selected from the group consisting of SEQ ID No. 1 to 23 and 47 to 52. In another aspect, one or more transgenes or recombinant DNA constructs encode a gene comprise a coding sequence having at least 50%, 55%, 60%, 65%, 70%, 75%, 80%, 85%, 90%, 95%, 97%, 98%, 99%, or 100% identity to a sequence selected from the group consisting of SEQ ID No. 24 to 46 and 53 to 58.

In one aspect, a recombinant DNA construct of the present disclosure comprises a promoter capable of driving gene transcription in a plant, operably linked to a polynucleotide encoding a polypeptide having at least 70%, at least 75%, at least 80%, at least 85%, at least 90%, at least 95%, at least 96%, at least 97%, at least 98%, at least 99% or 100% sequence identity to a polypeptide selected from the group consisting of SEQ ID NOs: 1 to 23 and 47 to 52. In one aspect, a recombinant DNA construct or expression cassette in a transgene provided herein comprises a promoter selected from the group consisting of a constitutive promoter, an inducible promoter, and a tissue-preferred promoter (for example, without being limiting, a leaf-specific promoter, a shoot-specific promoter, a root-specific promoter, or a meristem-specific promoter).

Exemplary constitutive promoters include the core promoter of the Rsyn7 promoter and other constitutive promoters disclosed in U.S. Pat. No. 6,072,050; the core CaMV 35S promoter (Odell et al. (1985) Nature 313:810-812); ubiquitin (Christensen et al. (1989) Plant Mol. Biol. 12:619-632 and Christensen et al. (1992) Plant Mol. Biol. 18:675-689); pEMU (Last et al. (1991) Theor. Appl. Genet. 81:581-588); MAS (Velten et al. (1984) EMBO J 3:2723-2730); ALS promoter (U.S. Pat. No. 5,659,026), and the like.

Exemplary chemical-inducible promoters include the tobacco PR-1a promoter, which is activated by salicylic acid. Other chemical-inducible promoters of interest include steroid-responsive promoters (see, for example, the glucocorticoid-inducible promoter in Schena et al. (1991) Proc. Natl. Acad. Sci. USA 88:10421-10425 and McNellis et al. (1998) Plant J. 14(2):247-257) and tetracycline-inducible promoters (see, for example, Gatz et al. (1991) Mol. Gen. Genet. 227:229-237, and U.S. Pat. Nos. 5,814,618 and 5,789,156). Additional exemplary promoters that can be used herein are those responsible for heat-regulated gene expression, light-regulated gene expression (for example, the pea rbcS-3A; the maize rbcS promoter; the chlorophyll alb-binding protein gene found in pea; or the Arabssu promoter), hormone-regulated gene expression (for example, the abscisic acid (ABA) responsive sequences from the Em gene of wheat; the ABA-inducible HVA1 and HVA22, and rd29A promoters of barley and Arabidopsis; and wound-induced gene expression (for example, of wunl), organ specific gene expression (for example, of the tuber-specific storage protein gene; the 23-kDa zein gene from maize described by; or the French bean (β-phascolin gene), or pathogen-inducible promoters (for example, the PR-1, prp-1, or (β-1,3 glucanase promoters, the fungal-inducible wirla promoter of wheat, and the nematode-inducible promoters, TobRB7-5A and Hmg-1, of tobacco arid parsley, respectively).

Additional exemplary tissue-preferred promoters include those disclosed in Yamamoto et al. (1997) Plant J. 12 (2): 255-265; Kawamata et al. (1997) Plant Cell Physiol. 38 (7): 792-803; Hansen et al. (1997) Mol. Gen. Genet. 254 (3): 337-343; Russell et al. (1997) Transgenic Res. 6 (2): 157-168; Rinchart et al. (1996) Plant Physiol. 112 (3): 1331-1341; Van Camp et al. (1996) Plant Physiol. 112 (2): 525-535; Canevascini et al. (1996) Plant Physiol. 112 (2): 513-524; Yamamoto et al. (1994) Plant Cell Physiol. 35 (5): 773-778; Lam (1994) Results Probl. Cell Differ. 20:181-196; Orozco et al. (1993) Plant Mol. Biol. 23 (6): 1129-1138; Matsuoka et al. (1993) Proc Natl. Acad. Sci. USA 90 (20): 9586-9590; and Guevara-Garcia et al. (1993) Plant J. 4 (3): 495-505.

As used herein, “operably linked” refers to a functional linkage between two or more elements. For example, an operable linkage between a polynucleotide of interest and a regulatory sequence (e.g., a promoter) is a functional link that allows for expression of the polynucleotide of interest. Operably linked elements may be contiguous or non-contiguous.

In one aspect, a transgene provided herein comprises a heterologous or non-tobacco promoter or coding sequence. In another aspect, a transgene provided herein comprises a endogenous or tobacco-origin promoter or coding sequence. As used herein, “heterologous” refers to a sequence that originates from a foreign species, or, if from the same species, is substantially modified from its native form in composition and/or genomic locus by deliberate human intervention. The term also is applicable to nucleic acid constructs, also referred to herein as “polynucleotide constructs” or “nucleotide constructs.” In this manner, a “heterologous” nucleic acid construct is intended to mean a construct that originates from a foreign species, or, if from the same species, is substantially modified from its native form in composition and/or genomic locus by deliberate human intervention. Heterologous nucleic acid constructs include, but are not limited to, recombinant nucleotide constructs that have been introduced into a plant or plant part thereof, for example, via transformation methods or subsequent breeding of a transgenic plant with another plant of interest.

In one aspect, a recombinant DNA construct, modified plant, seed, plant component, plant cell, or plant genome provided herein comprises a heterologous promoter operably linked to a polynucleotide encoding a polypeptide having at least 40%, at least 50%, at least 60%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity to a polypeptide selected from the group consisting of SEQ ID NOs: 24 to 46 and 53 to 58.

Enhancer elements are regions of DNA that can be bound by proteins to activate RNA transcription. In one aspect, a promoter sequence used herein is operably linked to an enhancer element. In one aspect, an enhancer element provided herein is a CsVMV promoter.

Also provided herein are the transformation of tobacco plants with recombinant constructs or expression cassettes described herein using any suitable transformation methods known in the art. Methods for introducing polynucleotide sequences into tobacco plants are known in the art and include, but are not limited to, stable transformation methods, transient transformation methods, and virus-mediated methods. “Stable transformation” refers to transformation where the nucleotide construct of interest introduced into a plant integrates into a genome of the plant and is capable of being inherited by the progeny thereof. “Transient transformation” is intended to mean that a sequence is introduced into the plant and is only temporally expressed or is only transiently present in the plant.

In one aspect, methods and compositions provided herein comprise the introduction of one or more polynucleotides into one or more plant cells. In one aspect, a plant genome provided herein is modified to include an introduced polynucleotide or recombinant DNA construct. As used herein, “plant genome” refers to a nuclear genome, a mitochondrial genome, or a plastid (e.g., chloroplast) genome of a plant cell. In another aspect, a polynucleotide provided herein is integrated into an artificial chromosome. In one aspect, an artificial chromosome comprising a polynucleotide provided herein is integrated into a plant cell.

In one aspect, transgenes provided herein comprise a recombinant DNA construct. In one aspect, recombinant DNA constructs or expression cassettes provided herein can comprise a selectable marker gene for the selection of transgenic cells. Selectable marker genes include, but are not limited to, genes encoding antibiotic resistance, such as those encoding neomycin phosphotransferase II (NEO) and hygromycin phosphotransferase (HPT), as well as genes conferring resistance to herbicidal compounds, such as glufosinate ammonium, bromoxynil, imidazolinones, triazolopyrimidines, sulfonylurea (e.g., chlorsulfuron and sulfometuron methyl), and 2,4-dichlorophenoxyacetate (2,4-D). Additional selectable markers include phenotypic markers such as β-galactosidase and fluorescent proteins such as green fluorescent protein (GFP).

In one aspect, methods and compositions provided herein comprise a vector. As used herein, the terms “vector” or “plasmid” are used interchangeably and refer to a circular, double-stranded DNA molecule that is physically separate from chromosomal DNA. In one aspect, a plasmid or vector used herein is capable of replication in vivo. A “transformation vector,” as used herein, is a plasmid that is capable of transforming a plant cell. In an aspect, a plasmid provided herein is a bacterial plasmid. In another aspect, a plasmid provided herein is an Agrobacterium Ti plasmid or derived from an Agrobacterium Ti plasmid.

In one aspect, a plasmid or vector provided herein is a recombinant vector. As used herein, the term “recombinant vector” refers to a vector formed by laboratory methods of genetic recombination, such as molecular cloning. In another aspect, a plasmid provided herein is a synthetic plasmid. As used herein, a “synthetic plasmid” is an artificially created plasmid that is capable of the same functions (e.g., replication) as a natural plasmid (e.g., Ti plasmid). Without being limited, one skilled in the art can create a synthetic plasmid de novo via synthesizing a plasmid by individual nucleotides, or by splicing together nucleic acid molecules from different pre-existing plasmids.

Vectors are commercially available or can be produced by recombinant DNA techniques routine in the art. A vector containing a nucleic acid can have expression elements operably linked to such a nucleic acid, and further can include sequences such as those encoding a selectable marker (e.g., an antibiotic resistance gene). A vector containing a nucleic acid can encode a chimeric or fusion polypeptide (i.e., a polypeptide operatively linked to a heterologous polypeptide, which can be at either the N-terminus or C-terminus of the polypeptide). Representative heterologous polypeptides are those that can be used in purification of the encoded polypeptide (e.g., 6×His tag, glutathione S-transferase (GST)).

Suitable methods of introducing polynucleotides (e.g., transgenes, recombinant vectors, recombinant DNA constructs, expression constructs) into plant cells of the present disclosure include microinjection (Crossway et al. (1986) Biotechniques 4:320-334), electroporation (Shillito et al. (1987) Meth. Enzymol. 153:313-336; Riggs et al. (1986) Proc. Natl. Acad. Sci. USA 83:5602-5606), Agrobacterium-mediated transformation (U.S. Pat. Nos. 5,104,310, 5,149,645, 5,177,010, 5,231,019, 5,463,174, 5,464,763, 5,469,976, 4,762,785, 5,004,863, 5,159,135, 5,563,055, and 5,981,840), direct gene transfer (Paszkowski et al. (1984) EMBO J. 3:2717-2722), and ballistic particle acceleration (see, for example, U.S. Pat. Nos. 4,945,050, 5,141,131, 5,886,244, 5,879,918, and 5,932,782; Tomes et al. (1995) in Plant Cell, Tissue, and Organ Culture Fundamental Methods, ed. Gamborg and Phillips (Springer-Verlag, Berlin); McCabe et al. (1988) Biotechnology 6:923-926). Also see Weissinger et al. (1988) Ann. Rev. Genet. 22:421-477; Christou et al. (1988) Plant Physiol. 87:671-674 (soybean); McCabe et al. (1988) Bio/Technology 6:923-926 (soybean); Finer and McMullen (1991) In Vitro Cell Dev. Biol. 27P: 175-182 (soybean); Singh et al. (1998) Theor. Appl. Genet. 96:319-324 (soybean); De Wet et al. (1985) in The Experimental Manipulation of Ovule Tissues, ed. Chapman et al. (Longman, N.Y.), pp. 197-209 (pollen); Kaeppler et al. (1990) Plant Cell Reports 9:415-418 and Kaeppler et al. (1992) Theor. Appl. Genet. 84:560-566 (whisker-mediated transformation); D'Halluin et al. (1992) Plant Cell 4:1495-1505 (electroporation). In one aspect, a bacterial cell provided herein comprises a recombinant DNA construct or recombinant vector provided herein.

In another aspect, recombinant constructs or expression cassettes provided herein may be introduced into plants by contacting plants with a virus or viral nucleic acids. Generally, such methods involve incorporating an expression cassette of the present disclosure within a viral DNA or RNA molecule. It is recognized that promoters for use in the expression cassettes provided herein also encompass promoters utilized for transcription by viral RNA polymerases. Methods for introducing polynucleotides into plants and expressing a protein encoded therein, involving viral DNA or RNA molecules, are known in the art. See, for example, U.S. Pat. Nos. 5,889,191, 5,889,190, 5,866,785, 5,589,367, 5,316,931, and Porta et al. (1996) Molecular Biotechnology 5:209-221.

Any plant tissue that can be subsequently propagated using clonal methods, whether by organogenesis or embryogenesis, may be transformed with a recombinant construct or an expression cassette provided herein. By “organogenesis” in intended the process by which shoots and roots are developed sequentially from meristematic centers. By “embryogenesis” is intended the process by which shoots and roots develop together in a concerted fashion (not sequentially), whether from somatic cells or gametes. Exemplary tissues that are suitable for various transformation protocols described herein include, but are not limited to, callus tissue, existing meristematic tissue (e.g., apical meristems, axillary buds, and root meristems) and induced meristem tissue (e.g., cotyledon meristem and hypocotyl meristem), hypocotyls, cotyledons, leaf disks, pollen, embryos, and the like.

It is understood that any modified tobacco plant of the present disclosure can further comprise additional agronomically desirable traits, for example, by transformation with a genetic construct or transgene using a technique known in the art. Without limitation, an example of a desired trait is herbicide resistance, pest resistance, disease resistance, high yield, high grade index value, curability, curing quality, mechanical harvestability, holding ability, leaf quality, height, plant maturation (e.g., early maturing, early to medium maturing, medium maturing, medium to late maturing, or late maturing), stalk size (e.g., a small, medium, or a large stalk), or leaf number per plant (e.g., a small (e.g., 5-10 leaves), medium (e.g., 11-15 leaves), or large (e.g., 16-21) number of leaves), or any combination. In one aspect, tobacco plants capable of producing cured leaf with reduced TSNA or seeds provided herein comprise one or more transgenes expressing one or more insecticidal proteins, such as, for example, a crystal protein of Bacillus thuringiensis or a vegetative insecticidal protein from Bacillus cereus, such as VIP3 (see, for example, Estruch et al. (1997) Nat. Biotechnol. 15:137). In another aspect, tobacco plants provided herein further comprise an introgressed trait conferring resistance to brown stem rot (U.S. Pat. No. 5,689,035) or resistance to cyst nematodes (U.S. Pat. No. 5,491,081).

The level and/or activity of polypeptides provided herein may be modulated by employing a polynucleotide that is not capable of directing, in a transformed plant, the expression of a protein or an RNA. For example, the polynucleotides of the invention may be used to design polynucleotide constructs that can be employed in methods for altering or mutating a genomic nucleotide sequence in an organism. Such polynucleotide constructs include, but are not limited to, RNA: DNA vectors, RNA: DNA mutational vectors, RNA: DNA repair vectors, mixed-duplex oligonucleotides, self-complementary RNA: DNA oligonucleotides and recombinogenic oligonucleobases. Such nucleotide constructs and methods of use are known in the art. See, U.S. Pat. Nos. 5,565,350; 5,731,181; 5,756,325; 5,760,012; 5,795,972 and 5,871,984; each of which is incorporated herein by reference as if set forth in its entirety. See also, International Patent Application Publication Nos. WO 98/149350, WO 99/107865 and WO 99/125921; and Bectham et al. (1999) Proc. Natl. Acad. Sci. USA 96:8774-8778; each of which is incorporated herein by reference as if set forth in its entirety.

The present disclosure also provides compositions and methods for inhibiting the expression or function of one or more polypeptides that suppress, directly or indirectly, the production or accumulation of one or more antioxidants in a plant, particularly plants of the Nicotiana tabacum genus, including tobacco plants of various commercial varieties.

In one aspect, inhibition of the expression of one or more polypeptides provided herein may be obtained by RNA interference (RNAi) by expression of a transgene capable of producing an inhibitory sequence provided herein. In one aspect, RNAi comprises expressing a non-coding RNA. As used herein, a “non-coding RNA” is selected from the group consisting of a microRNA (miRNA), a small interfering RNA (siRNA), a trans-acting siRNA (ta-siRNA), a transfer RNA (tRNA), a ribosomal RNA (rRNA), an intron, a hairpin RNA (hpRNA), and an intron-containing hairpin RNA (ihpRNA). In one aspect, a single non-coding RNA provided herein inhibits the expression of at least 1, at least 2, at least 3, at least 4, at least 5, at least 6, at least 7, at least 8, at least 9, at least 10, or more than 10 polypeptides. In one aspect, a non-coding RNA provided herein is stably transformed into a plant genome. In another aspect, a non-coding RNA provided herein is transiently transformed into a plant genome.

As used herein, the terms “suppress,” “inhibit,” “inhibition,” “inhibiting”, and “downregulation” are defined as any method known in the art or described herein that decreases the expression or function of a gene product (e.g., an mRNA, a protein, a non-coding RNA). “Inhibition” can be in the context of a comparison between two cells, for example, a modified cell versus a control cell. Inhibition of expression or function of a gene product can also be in the context of a comparison between plant cells, organelles, organs, tissues, or plant components within the same plant or between different plants, and includes comparisons between developmental or temporal stages within the same plant or plant component or between plants or plant components. “Inhibition” includes any relative decrement of function or production of a gene product of interest, up to and including complete elimination of function or production of that gene product. The term “inhibition” encompasses any method or composition that down-regulates translation and/or transcription of the target gene product or functional activity of the target gene product. “Inhibition” need not comprise complete elimination of expression of a gene product. In an aspect, a gene product in a modified cell provided herein comprises expression that is at least 1%, at least 2%, at least 3%, at least 4%, at least 5%, at least 10%, at least 20%, at least 25%, at least 30%, at least 40%, at least 50%, at least 60%, at least 70%, at least 80%, at least 90%, at least 95%, or 100% lower than the expression of the gene product in a control cell. In another aspect, a gene product in a modified cell provided herein comprises expression that is between 1% and 100%, between 1% and 95%, between 1% and 90%, between 1% and 80%, between 1% and 70%, between 1% and 60%, between 1% and 50%, between 1% and 40%, between 1% and 30%, between 1% and 25%, between 1% and 20%, between 1% and 15%, between 1% and 10%, between 1% and 5%, between 5% and 25%, between 5% and 50%, between 5% and 75%, between 5% and 100%, between 10% and 25%, between 10% and 50%, between 10% and 75%, between 10% and 100%, between 25% and 50%, between 25% and 75%, between 25% and 100%, or between 50% and 100% lower than the expression of the gene product in a control cell.

As used herein, a “target site” refers to a location of a polynucleotide sequence that is bound to and cleaved by a site-specific nuclease introducing a double stranded break into the nucleic acid backbone. In another aspect a target site comprises at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 29, or at least 30 consecutive nucleotides. In another aspect, a target site provided herein is at least 10, at least 20, at least 30, at least 40, at least 50, at least 75, at least 100, at least 125, at least 150, at least 200, at least 250, at least 300, at least 400, or at least 500 nucleotides. In one aspect a site-specific nuclease binds to a target site. In another aspect a site-specific nuclease binds to a target site via a guiding non-coding RNA (i.e., such as, without being limiting, a CRISPR RNA or single-guide RNA (both described in detail below)). In one aspect, a non-coding RNA provided herein is complementary to a target site. It will be appreciated that perfect complementarity is not required for a non-coding RNA to bind to a target site; at least 1, at least 2, at least 3, at least 4, or at least 5, at least 6, at least 7 or at least 8 mismatches between a target site and a non-coding RNA can be tolerated. As used herein, a “target region” or a “targeted region” refers to a polynucleotide sequence that is desired to be modified. In one aspect, a “target region,” “targeted region,” or a “target gene” is flanked by two or more, three or more, four or more, five or more, six or more, seven or more, eight or more, nine or more, or ten or more target sites. A “target gene” refers to a polynucleotide sequence encoding a gene that is desired to be modified or from which transcript expression is desired to be modulated. In one aspect, a polynucleotide sequence comprising a target gene further comprises one or more target sites. In another aspect, a transgene is said to be targeting a target site or a target gene. In another aspect, a target region comprises one or more, two or more, three or more, four or more, five or more, six or more, seven or more, eight or more, nine or more, or ten or more target genes. Without being limiting, in one aspect a target region can be subject to deletion or inversion. As used herein, “flanked” when used to describe a target region, refers to two or more target sites physically surrounding the target region, with one target site on each side of the target region.

As used herein, in the context of a transgene “directly modulating” or “directly modulates” refers to inducing a change in the transcript or protein level of a target gene by an agent produced by the transgene and sharing sufficient homology with at least a portion of the target gene. Direct modulation can result in a change in transcriptional activity, transcript stability, transcript constitution, or transcript expression level which can either increase or decrease the number of transcripts available for translation and can either increase or decrease the number of protein molecules.

A target site can be positioned in a polynucleotide sequence encoding a leader, an enhancer, a transcriptional start site, a promoter, a 5′-UTR, an exon, an intron, a 3′-UTR, a polyadenylation site, or a termination sequence. It will be appreciated that a target site can be also be positioned upstream or downstream of a sequence encoding a leader, an enhancer, a transcriptional start site, a promoter, a 5′-UTR, an exon, an intron, a 3′-UTR, a polyadenylation site, or a termination sequence. In one aspect, a target site is positioned within 10, within 20, within 30, within 40, within 50, within 75, within 100, within 125, within 150, within 200, within 250, within 300, within 400, within 500, within 600, within 700, within 800, within 900, within 1000, within 1250, within 1500, within 2000, within 2500, within 5000, within 10,000, or within 25,000 nucleotides of a polynucleotide encoding a leader, an enhancer, a transcriptional start site, a promoter, a 5′-UTR, an exon, an intron, a 3′-UTR, a polyadenylation site, a gene, or a termination sequence.

As used herein, “upstream” refers to a nucleic acid sequence that is positioned before the 5′ end of a linked nucleic acid sequence. As used herein, “downstream” refers to a nucleic acid sequence is positioned after the 3′ end of a linked nucleic acid sequence. As used herein, “5′” refers to the start of a coding DNA sequence or the beginning of an RNA molecule. As used herein, “3′” refers to the end of a coding DNA sequence or the end of an RNA molecule. It will be appreciated that an “inversion” refers to reversing the orientation of a given polynucleotide sequence. For example, if the sample sequence 5′-ATGATC-3′ is inverted it will read 5′-CTAGTA-3′ in reverse orientation. Additionally, the sample sequence 5′-ATGATC-3′ is considered to be in “opposite orientation” to the sample sequence 5′-CTAGTA-3′.

The term “inhibitory sequence” encompasses any polynucleotide or polypeptide sequence capable of inhibiting the expression or function of a gene in a plant, such as full-length polynucleotide or polypeptide sequences, truncated polynucleotide or polypeptide sequences, fragments of polynucleotide or polypeptide sequences, variants of polynucleotide or polypeptide sequences, sense-oriented nucleotide sequences, antisense-oriented nucleotide sequences, the complement of a sense- or antisense-oriented nucleotide sequence, inverted regions of nucleotide sequences, hairpins of nucleotide sequences, double-stranded nucleotide sequences, single-stranded nucleotide sequences, combinations thereof, and the like. The term “polynucleotide sequence” includes sequences of RNA, DNA, chemically modified nucleic acids, nucleic acid analogs, combinations thereof, and the like.

Inhibitory sequences are designated herein by the name of the target gene product. Thus, as a non-limiting example, an “gene X inhibitory sequence” refers to an inhibitory sequence that is capable of inhibiting the expression of a gene X locus in a plant, for example, at the level of transcription and/or translation, or which is capable of inhibiting the function of a gene product. When the phrase “capable of inhibiting” is used in the context of a transgene containing polynucleotide inhibitory sequence, it is intended to mean that the inhibitory sequence itself exerts the inhibitory effect; or, where the inhibitory sequence encodes an inhibitory nucleotide molecule (for example, hairpin RNA, miRNA, or double-stranded RNA polynucleotides), or encodes an inhibitory polypeptide (e.g., a polypeptide that inhibits expression or function of the target gene product), following its transcription (for example, in the case of an inhibitory sequence encoding a hairpin RNA, miRNA, or double-stranded RNA polynucleotide) or its transcription and translation (in the case of an inhibitory sequence encoding an inhibitory polypeptide), the transcribed or translated product, respectively, exerts the inhibitory effect on the target gene product (e.g., inhibits expression or function of the target gene product).

An inhibitory sequence provided herein can be a sequence triggering gene silencing via any silencing pathway or mechanism known in the art, including, but not limited to, sense suppression/co-suppression, antisense suppression, double-stranded RNA (dsRNA) interference, hairpin RNA interference and intron-containing hairpin RNA interference, amplicon-mediated interference, ribozymes, small interfering RNA, artificial or synthetic microRNA, and artificial trans-acting siRNA.

One aspect of the present application relates to methods of screening and selecting cells for targeted edits and methods of selecting cells comprising targeted edits. Nucleic acids can be isolated using various techniques. For example, nucleic acids can be isolated using any method including, without limitation, recombinant nucleic acid technology, and/or the polymerase chain reaction (PCR). General PCR techniques are described, for example in PCR Primer: A Laboratory Manual, Dieffenbach & Dveksler, Eds., Cold Spring Harbor Laboratory Press, 1995. Recombinant nucleic acid techniques include, for example, restriction enzyme digestion and ligation, which can be used to isolate a nucleic acid. Isolated nucleic acids also can be chemically synthesized, either as a single nucleic acid molecule or as a series of oligonucleotides. Polypeptides can be purified from natural sources (e.g., a biological sample) by known methods such as DEAE ion exchange, gel filtration, and hydroxyapatite chromatography. A polypeptide also can be purified, for example, by expressing a nucleic acid in an expression vector. In addition, a purified polypeptide can be obtained by chemical synthesis. The extent of purity of a polypeptide can be measured using any appropriate method, e.g., column chromatography, polyacrylamide gel electrophoresis, or HPLC analysis.

Also provided herein is cured tobacco material made from tobacco leaf, tobacco plants, or plant components provided herein. “Curing” is the aging process that reduces moisture and brings about the destruction of chlorophyll giving tobacco leaf a golden color and by which starch is converted to sugar. Cured tobacco therefore has a higher reducing sugar content and a lower starch content compared to harvested green leaf. In one aspect, tobacco plants or plant components provided herein can be cured using conventional means, e.g., flue-cured, barn-cured, fire-cured, air-cured or sun-cured. See, for example, Tso (1999, Chapter 1 in Tobacco, Production, Chemistry and Technology, Davis & Nielsen, eds., Blackwell Publishing, Oxford) for a description of different types of curing methods. Cured tobacco is usually aged in a wooden drum (e.g., a hogshead) or cardboard cartons in compressed conditions for several years (e.g., two to five years), at a moisture content ranging from 10% to about 25%. See, U.S. Pat. Nos. 4,516,590 and 5,372,149. Cured and aged tobacco then can be further processed. Further processing includes conditioning the tobacco under vacuum with or without the introduction of steam at various temperatures, pasteurization, and fermentation. Fermentation typically is characterized by high initial moisture content, heat generation, and a 10 to 20% loss of dry weight. See, for example, U.S. Pat. Nos. 4,528,993, 4,660,577, 4,848,373, 5,372,149; U.S. Publication No. 2005/0178398; and Tso (1999, Chapter 1 in Tobacco, Production, Chemistry and Technology, Davis & Nielsen, eds., Blackwell Publishing, Oxford). Cured, aged, and fermented tobacco can be further processed (e.g., cut, shredded, expanded, or blended). See, for example, U.S. Pat. Nos. 4,528,993; 4,660,577; and 4,987,907. In one aspect, the cured tobacco material of the present disclosure is flue-cured, sun-cured, air-cured, or fire-cured.

Tobacco material obtained from modified tobacco lines, varieties or hybrids of the present disclosure can be used to make tobacco products. As used herein, “tobacco product” is defined as any product made or derived from tobacco that is intended for human use or consumption. In an aspect, a tobacco product provided herein comprises cured components from a modified tobacco plant provided herein. In another aspect, a tobacco product provided herein comprises cured tobacco leaf from a modified tobacco plant provided herein.

Tobacco products provided herein include, without limitation, cigarette products (e.g., cigarettes, bidi cigarettes, kreteks), cigar products (e.g., cigars, cigar wrapping tobacco, cigarillos), pipe tobacco products, products derived from tobacco, tobacco-derived nicotine products, smokeless tobacco products (e.g., moist snuff, dry snuff, snus, chewing tobacco, moist smokeless tobacco, fine cut chewing tobacco, long cut chewing tobacco, pouched chewing tobacco), films, chewables (e.g., gum), lozenges, dissolving strips, tabs, tablets, shaped parts, gels, consumable units, insoluble matrices, hollow shapes, reconstituted tobacco, expanded tobacco, and the like. See, for example, U.S. Patent Publication No. US 2006/0191548.

As used herein, “cigarette” refers a tobacco product having a “rod” and “filler”. The cigarette “rod” includes the cigarette paper, filter, plug wrap (used to contain filtration materials), tipping paper that holds the cigarette paper (including the filler) to the filter, and all glues that hold these components together. The “filler” includes (1) all tobaccos, including but not limited to reconstituted and expanded tobacco, (2) non-tobacco substitutes (including but not limited to herbs, non-tobacco plant materials and other spices that may accompany tobaccos rolled within the cigarette paper), (3) casings, (4) flavorings, and (5) all other additives (that are mixed into tobaccos and substitutes and rolled into the cigarette).

In one aspect, this disclosure provides nicotine derived from and a method of producing nicotine from a modified tobacco plant provided herein for use in a product.

In one aspect, a method provided herein comprises preparing a tobacco product using cured tobacco leaf from a modified tobacco plant provided herein.

As used herein, “reconstituted tobacco” refers to a part of tobacco filler made from tobacco dust and other tobacco scrap material, processed into sheet form and cut into strips to resemble tobacco. In addition to the cost savings, reconstituted tobacco is very important for its contribution to cigarette taste from processing flavor development using reactions between ammonia and sugars.

As used herein, “expanded tobacco” refers to a part of tobacco filler which is processed through expansion of suitable gases so that the tobacco is “puffed” resulting in reduced density and greater filling capacity. It reduces the weight of tobacco used in cigarettes.

Tobacco products derived from plants of the present disclosure also include cigarettes and other smoking articles, particularly those smoking articles including filter elements, where the rod of smokeable material includes cured tobacco within a tobacco blend. In an aspect, a tobacco product of the present disclosure is selected from the group consisting of a cigarillo, a non-ventilated recess filter cigarette, a vented recess filter cigarette, a bidi cigarette, a cigar, snuff, pipe tobacco, cigar tobacco, cigarette tobacco, chewing tobacco, leaf tobacco, hookah tobacco, shredded tobacco, and cut tobacco. In another aspect, a tobacco product of the present disclosure is a smokeless tobacco product. Smokeless tobacco products are not combusted and include, but not limited to, chewing tobacco, moist smokeless tobacco, snus, and dry snuff. Chewing tobacco is coarsely divided tobacco leaf that is typically packaged in a large pouch-like package and used in a plug or twist. Moist smokeless tobacco is a moist, more finely divided tobacco that is provided in loose form or in pouch form and is typically packaged in round cans and used as a pinch or in a pouch placed between an adult tobacco consumer's cheek and gum. Snus is a heat treated smokeless tobacco. Dry snuff is finely ground tobacco that is placed in the mouth or used nasally. In a further aspect, a tobacco product of the present disclosure is selected from the group consisting of loose leaf chewing tobacco, plug chewing tobacco, moist snuff, and nasal snuff. In yet another aspect, a tobacco product of the present disclosure is selected from the group consisting of an electronically heated cigarette, an e-cigarette, an electronic vaporing device.

The present disclosure further provides a method for manufacturing a tobacco product comprising tobacco material from tobacco plants provided herein. In one aspect, methods provided herein comprise conditioning aged tobacco material made from tobacco plants provided herein to increase its moisture content from between about 12.5% and about 13.5% to about 21%, blending the conditioned tobacco material to produce a desirable blend. In one aspect, the method of manufacturing a tobacco product provided herein further comprises casing or flavoring the blend. Generally, during the casing process, casing or sauce materials are added to blends to enhance their quality by balancing the chemical composition and to develop certain desired flavor characteristics. Further details for the casing process can be found in Tobacco Production, Chemistry and Technology, Edited by L. Davis and M. Nielsen, Blackwell Science, 1999.

Tobacco material provided herein can be also processed using methods including, but not limited to, heat treatment (e.g., cooking, toasting), flavoring, enzyme treatment, expansion and/or curing. Both fermented and non-fermented tobaccos can be processed using these techniques. Examples of suitable processed tobaccos include dark air-cured, dark fire cured, burley, flue cured, and cigar filler or wrapper, as well as the products from the whole leaf stemming operation. In one aspect, tobacco fibers include up to 70% dark tobacco on a fresh weight basis. For example, tobacco can be conditioned by heating, sweating and/or pasteurizing steps as described in U.S. Publication Nos. 2004/0118422 or 2005/0178398.

Tobacco material provided herein can be subject to fermentation. Fermenting typically is characterized by high initial moisture content, heat generation, and a 10 to 20% loss of dry weight. See, e.g., U.S. Pat. Nos. 4,528,993; 4,660,577; 4,848,373; and 5,372,149. In addition to modifying the aroma of the leaf, fermentation can change either or both the color and texture of a leaf. Also during the fermentation process, evolution gases can be produced, oxygen can be taken up, the pH can change, and the amount of water retained can change. See, for example, U.S. Publication No. 2005/0178398 and Tso (1999, Chapter 1 in Tobacco, Production, Chemistry and Technology, Davis & Nielsen, eds., Blackwell Publishing, Oxford). Cured, or cured and fermented tobacco can be further processed (e.g., cut, expanded, blended, milled or comminuted) prior to incorporation into the oral product. The tobacco, in some cases, is long cut fermented cured moist tobacco having an oven volatiles content of between 48 and 50 weight percent prior to mixing with a copolymer and, optionally, flavorants and other additives.

In one aspect, tobacco material provided herein can be processed to a desired size. In certain aspects, tobacco fibers can be processed to have an average fiber size of less than 200 micrometers. In one aspect, tobacco fibers are between 75 and 125 micrometers. In another aspect, tobacco fibers are processed to have a size of 75 micrometers or less. In one aspect, tobacco fibers include long cut tobacco, which can be cut or shredded into widths of about 10 cuts/inch up to about 110 cuts/inch and lengths of about 0.1 inches up to about 1 inch. Double cut tobacco fibers can have a range of particle sizes such that about 70% of the double cut tobacco fibers falls between the mesh sizes of −20 mesh and 80 mesh.

Tobacco material provided herein can be processed to have a total oven volatiles content of about 10% by weight or greater; about 20% by weight or greater; about 40% by weight or greater; about 15% by weight to about 25% by weight; about 20% by weight to about 30% by weight; about 30% by weight to about 50% by weight; about 45% by weight to about 65% by weight; or about 50% by weight to about 60% by weight. Those of skill in the art will appreciate that “moist” tobacco typically refers to tobacco that has an oven volatiles content of between about 40% by weight and about 60% by weight (e.g., about 45% by weight to about 55% by weight, or about 50% by weight). As used herein, “oven volatiles” are determined by calculating the percentage of weight loss for a sample after drying the sample in a pre-warmed forced draft oven at 110° C. for 3.25 hours. An oral product can have a different overall oven volatiles content than the oven volatiles content of the tobacco fibers used to make the oral product. The processing steps described herein can reduce or increase the oven volatiles content.

In one aspect, tobacco plants, seeds, plant components, plant cells, and plant genomes provided herein are from a tobacco type selected from the group consisting of flue-cured tobacco, sun-cured tobacco, air-cured tobacco, dark air-cured tobacco, and dark fire-cured tobacco. In another aspect, tobacco plants, seeds, plant components, plant cells, and plant genomes provided herein are from a tobacco type selected from the group consisting of Burley tobacco, Maryland tobacco, bright tobacco, Virginia tobacco, Oriental tobacco, Turkish tobacco, dark tobacco, and Galpão tobacco. In one aspect, a tobacco plant or seed provided herein is a hybrid plant or seed. As used herein, a “hybrid” is created by crossing two plants from different varieties or species, such that the progeny comprises genetic material from each parent. Skilled artisans recognize that higher order hybrids can be generated as well. For example, a first hybrid can be made by crossing Variety C with Variety D to create a C×D hybrid, and a second hybrid can be made by crossing Variety E with Variety F to create an E×F hybrid. The first and second hybrids can be further crossed to create the higher order hybrid (C×D)×(E×F) comprising genetic information from all four parent varieties.

Flue-cured tobaccos (also called Virginia or bright tobaccos) amount to approximately 40% of world tobacco production. Flue-cured tobaccos are often also referred to as “bright tobacco” because of the golden-yellow to deep-orange color it reaches during curing. Flue-cured tobaccos have a light, bright aroma and taste. Flue-cured tobaccos are generally high in sugar and low in oils. Major flue-cured tobacco growing countries are Argentina, Brazil, China, India, Tanzania and the U.S. In one aspect, modified tobacco plants or seeds provided herein are in a flue-cured tobacco background selected from the group consisting of CC 13, CC 27, CC 33, CC35, CC 37, CC 65, CC 67, CC 700, GF 318, GL 338, GL 368, GL 939, K 346, K 399, K326, NC 102, NC 196, NC 291, NC 297, NC 299, NC 471, NC 55, NC 606, NC 71, NC 72, NC 92, PVH 1118, PVH 1452, PVH 2110, SPEIGHT 168, SPEIGHT 220, SPEIGHT 225, SPEIGHT 227, SPEIGHT 236, and any variety essentially derived from any one of the foregoing varieties. In another aspect, modified tobacco plants or seeds provided herein are in a flue-cured tobacco background selected from the group consisting of Coker 48, Coker 176, Coker 371-Gold, Coker 319, Coker 347, GL 939, K 149, K326, K 340, K 346, K 358, K 394, K 399, K 730, NC 27NF, NC 37NF, NC 55, NC 60, NC 71, NC 72, NC 82, NC 95, NC 297, NC 606, NC 729, NC 2326, McNair 373, McNair 944, Ox 207, Ox 414 NF, Reams 126, Reams 713, Reams 744, RG 8, RG 11, RG 13, RG 17, RG 22, RG 81, RG H4, RG H51, Speight H-20, Speight G-28, Speight G-58, Speight G-70, Speight G-108, Speight G-111, Speight G-117, Speight 168, Speight 179, Speight NF-3, Va 116, Va 182, and any variety essentially derived from any one of the foregoing varieties. See WO 2004/041006 A1. In further aspects, modified tobacco plants, seeds, hybrids, varieties, or lines provided herein are in any flue cured background selected from the group consisting of K326, K346, and NC196.

Air-cured tobaccos include Burley, Maryland, and dark tobaccos. The common factor is that curing is primarily without artificial sources of heat and humidity. Burley tobaccos are light to dark brown in color, high in oil, and low in sugar. Burley tobaccos are air-cured in barns. Major Burley growing countries are Argentina, Brazil, Italy, Malawi, and the U.S. Maryland tobaccos are extremely fluffy, have good burning properties, low nicotine and a neutral aroma. Major Maryland growing countries include the U.S. and Italy. In one aspect, modified tobacco plants or seeds provided herein are in a Burley tobacco background selected from the group consisting of Clay 402, Clay 403, Clay 502, Ky 14, Ky 907, Ky 910, Ky 8959, NC 2, NC 3, NC 4, NC 5, NC 2000, TN 86, TN 90, TN 97, R 610, R 630, R 711, R 712, NCBH 129, HB4488PLC, PD 7319LC, Bu 21×Ky 10, HB04P, Ky 14×L 8, Kt 200, Newton 98, Pedigo 561, Pf561 and Va 509. In further aspects, modified tobacco plants, seeds, hybrids, varieties, or lines provided herein are in any Burley background selected from the group consisting of TN 90, KT 209, KT 206, KT212, and HB 4488. In another aspect, modified tobacco plants or seeds provided herein are in a Maryland tobacco background selected from the group consisting of Md 10, Md 40, Md 201, Md 609, Md 872 and Md 341.

Dark air-cured tobaccos are distinguished from other types primarily by its curing process which gives dark air-cured tobacco its medium- to dark-brown color and distinct aroma. Dark air-cured tobaccos are mainly used in the production of smokeless tobacco products including chewing tobacco and snuff. In one aspect, modified tobacco plants or seeds provided herein are in a dark air-cured tobacco background selected from the group consisting of Sumatra, Jatim, Dominican Cubano, Besuki, One sucker, Green River, Virginia sun-cured, and Paraguan Passado.

Dark fire-cured tobaccos are generally cured with low-burning wood fires on the floors of closed curing barns. Dark fire-cured tobaccos are used for making pipe blends, cigarettes, chewing tobacco, snuff and strong-tasting cigars. Major growing regions for dark fire-cured tobaccos are Tennessee, Kentucky, and Virginia, USA. In one aspect, modified tobacco plants or seeds provided herein are in a dark fire-cured tobacco background selected from the group consisting of Narrow Leaf Madole, Improved Madole, Tom Rosson Madole, Newton's VH Madole, Little Crittenden, Green Wood, Little Wood, Small Stalk Black Mammoth, DT 508, DT 518, DT 592, KY 171, DF 911, DF 485, TN D94, TN D950, VA 309, and VA 359.

Oriental tobaccos are also referred to as Greek, aroma and Turkish tobaccos due to the fact that they are typically grown in eastern Mediterranean regions such as Turkey, Greece, Bulgaria, Macedonia, Syria, Lebanon, Italy, and Romania. The small plant and leaf size, characteristic of today's Oriental varieties, as well as its unique aroma properties are a result of the plant's adaptation to the poor soil and stressful climatic conditions in which it develop over many past centuries. In one aspect, modified tobacco plants or seeds provided herein are in an Oriental tobacco background selected from the group consisting of Izmir, Katerini, Samsun, Basma and Krumovgrad, Trabzon, Thesalian, Tasova, Sinop, Izmit, Hendek, Edirne, Semdinli, Adiyanman, Yayladag, Iskenderun, Duzce, Macedonian, Mavra, Prilep, Bafra, Bursa, Bucak, Bitlis, Balikesir, and any variety essentially derived from any one of the foregoing varieties.

In one aspect, modified tobacco plants, seeds, hybrids, varieties, or lines provided herein are essentially derived from or in the genetic background of BU 64, CC 101, CC 200, CC 13, CC 27, CC 33, CC 35, CC 37, CC 65, CC 67, CC 301, CC 400, CC 500, CC 600, CC 700, CC 800, CC 900, CC 1063, Coker 176, Coker 319, Coker 371 Gold, Coker 48, CU 263, DF911, Galpão, GL 26H, GL 338, GL 350, GL 395, GL 600, GL 737, GL 939, GL 973, GF 157, GF 318, RJR 901, HB 04P, K 149, K 326, K 346, K 358, K394, K 399, K 730, NC 196, NC 37NF, NC 471, NC 55, NC 92, NC2326, NC 95, NC 925, PVH 1118, PVH 1452, PVH 2110, PVH 2254, PVH 2275, VA 116, VA 119, KDH 959, KT 200, KT204LC, KY 10, KY 14, KY 160, KY 17, KY 171, KY 907, KY 907LC, KTY14×L8 LC, Little Crittenden, McNair 373, McNair 944, male sterile KY 14×L8, Narrow Leaf Madole, MS KY171, Narrow Leaf Madole (phph), MS Narrow Leaf Madole, MS TND950, PD 7302LC, PD 7305LC, PD 7309LC, PD 7312LC, PD 7318LC, PD 7319LC, MSTKS 2002, TKF 2002, TKF 6400, TKF 4028, TKF 4024, KT206LC, KT209LC, KT210LC, KT212LC, NC 100, NC 102, NC 2000, NC 291, NC 297, NC 299, NC 3, NC 4, NC 5, NC 6, NC7, NC 606, NC 71, NC 72, NC 810, NC BH 129, NC 2002, Neal Smith Madole, OXFORD 207, ‘Perique’, PVH03, PVH09, PVH19, PVH50, PVH51, R 610, R 630, R 7-11, R 7-12, RG 17, RG 81, RG H51, RGH 4, RGH 51, RS 1410, Speight 168, Speight 172, Speight 179, Speight 210, Speight 220, Speight 225, Speight 227, Speight 234, Speight G-28, Speight G-70, Speight H-6, Speight H20, Speight NF3, TI 1406, TI 1269, TN 86, TN86LC, TN 90, TN90LC, TN 97, TN97LC, TN D94, TN D950, a TR (Tom Rosson) Madole, VA 309, VA 359, or any commercial tobacco variety according to standard tobacco breeding techniques known in the art.

All foregoing mentioned specific varieties of dark air-cured, Burley, Maryland, dark fire-cured, or Oriental type are only listed for exemplary purposes. Any additional dark air-cured, Burley, Maryland, dark fire-cured, or Oriental varieties are also contemplated in the present application.

Also provided herein are populations of tobacco plants described herein. In one aspect, a population of tobacco plants provided herein has a planting density of between about 5,000 and about 8000, between about 5,000 and about 7,600, between about 5,000 and about 7,200, between about 5,000 and about 6,800, between about 5,000 and about 6,400, between about 5,000 and about 6,000, between about 5,000 and about 5,600, between about 5,000 and about 5,200, between about 5,200 and about 8,000, between about 5,600 and about 8,000, between about 6,000 and about 8,000, between about 6,400 and about 8,000, between about 6,800 and about 8,000, between about 7,200 and about 8,000, or between about 7,600 and about 8,000 plants per acre. In another aspect, a population of tobacco plants provided herein is in a soil type with low to medium fertility.

Also provided herein are containers of seeds from tobacco plants described herein. A container of tobacco seeds of the present disclosure may contain any number, weight, or volume of seeds. For example, a container can contain at least, or greater than, about 100, at least, or greater than, about 200, at least, or greater than, about 300, at least, or greater than, about 400, at least, or greater than, about 500, at least, or greater than, about 600, at least, or greater than, about 700, at least, or greater than, about 800, at least, or greater than, about 900, at least, or greater than, about 1000, at least, or greater than, about 1500, at least, or greater than, about 2000, at least, or greater than, about 2500, at least, or greater than, about 3000, at least, or greater than, about 3500, at least, or greater than, or about 4000 or more seeds. Alternatively, the container can contain at least, or greater than, about 1 ounce, at least, or greater than, about 5 ounces, at least, or greater than, about 10 ounces, at least, or greater than, about 1 pound, at least, or greater than, about 2 pounds, at least, or greater than, about 3 pounds, at least, or greater than, about 4 pounds, at least, or greater than, about 5 pounds or more seeds. Containers of tobacco seeds may be any container available in the art. By way of non-limiting example, a container may be a box, a bag, a packet, a pouch, a tape roll, a tube, or a bottle.

The present disclosure also provides methods for breeding tobacco lines, cultivars, or varieties comprising cured leaf with reduced or eliminated TSNAs (and, optionally, also comprising increased antioxidants or decreased nitrite). Breeding can be carried out via any known procedures. DNA fingerprinting, SNP mapping, haplotype mapping or similar technologies may be used in a marker-assisted selection (MAS) breeding program to transfer or breed a desirable trait or allele into a tobacco plant. For example, a breeder can create segregating populations in an F₂ or backcross generation using F₁ hybrid plants provided herein or further crossing the F₁ hybrid plants with other donor plants with an agronomically desirable genotype. Plants in the F₂ or backcross generations can be screened for a desired agronomic trait or a desirable chemical profile using one of the techniques known in the art or listed herein. Depending on the expected inheritance pattern or the MAS technology used, self-pollination of selected plants before each cycle of backcrossing to aid identification of the desired individual plants can be performed. Backcrossing or other breeding procedure can be repeated until the desired phenotype of the recurrent parent is recovered. In one aspect, a recurrent parent in the present disclosure can be a flue-cured variety, a Burley variety, a dark air-cured variety, a dark fire-cured variety, or an Oriental variety. In another aspect, a recurrent parent can be a modified tobacco plant, line, or variety. Other breeding techniques can be found, for example, in Wernsman, E. A., and Rufty, R. C. 1987. Chapter Seventeen. Tobacco. Pages 669-698 In: Cultivar Development. Crop Species. W. H. Fchr (ed.), MacMillan Publishing Go., Inc., New York, N.Y., incorporated herein by reference in their entirety.

Results of a plant breeding program using modified tobacco plants described herein includes useful lines, cultivars, varieties, progeny, inbreds, and hybrids of the present disclosure. As used herein, the term “variety” refers to a population of plants that share constant characteristics which separate them from other plants of the same species. A variety is often, although not always, sold commercially. While possessing one or more distinctive traits, a variety is further characterized by a very small overall variation between individuals within that variety. A “pure line” variety may be created by several generations of self-pollination and selection, or vegetative propagation from a single parent using tissue or cell culture techniques. A variety can be essentially derived from another line or variety. As defined by the International Convention for the Protection of New Varieties of Plants (Dec. 2, 1961, as revised at Geneva on Nov. 10, 1972; on Oct. 23, 1978; and on Mar. 19, 1991), a variety is “essentially derived” from an initial variety if: a) it is predominantly derived from the initial variety, or from a variety that is predominantly derived from the initial variety, while retaining the expression of the essential characteristics that result from the genotype or combination of genotypes of the initial variety; b) it is clearly distinguishable from the initial variety; and c) except for the differences which result from the act of derivation, it conforms to the initial variety in the expression of the essential characteristics that result from the genotype or combination of genotypes of the initial variety. Essentially derived varieties can be obtained, for example, by the selection of a natural or induced mutant, a somaclonal variant, a variant individual from plants of the initial variety, backcrossing, or transformation. A first tobacco variety and a second tobacco variety from which the first variety is essentially derived, are considered as having essentially identical genetic background. A “line” as distinguished from a variety most often denotes a group of plants used non-commercially, for example in plant research. A line typically displays little overall variation between individuals for one or more traits of interest, although there may be some variation between individuals for other traits.

In one aspect, the present disclosure provides a method of producing a tobacco plant comprising crossing at least one tobacco plant of a first tobacco variety with at least one tobacco plant of a second tobacco variety, where the at least one tobacco plant of the first tobacco variety comprising one or more desired traits, e.g., comprising a reduced level of one or more tobacco-specific nitrosamines (TSNAs) in cured leaf and further comprising one or more traits selected from the group consisting of: a reduced level of nitrite, an increased level of oxygen radical absorbance capacity (ORAC), and an increased level of one or more antioxidants, wherein said reduced or increased level is compared to a control tobacco plant of the same cross grown and cured under comparable conditions; and selecting for progeny tobacco plants that exhibit the one or more desired traits. In one aspect, a first tobacco variety provided herein comprises modified tobacco plants. In another aspect, a second tobacco variety provided herein comprises modified tobacco plants. In one aspect, a first or second tobacco variety is male sterile. In another aspect, a first or second tobacco variety is cytoplasmically male sterile. In another aspect, a first or second tobacco variety is female sterile. In one aspect, a first or second tobacco variety is an elite variety. In another aspect, a first or second tobacco variety is a hybrid.

In one aspect, the present disclosure provides a method of introgressing one or more transgenes or mutations into a tobacco variety, the method comprising: (a) crossing a first tobacco variety comprising one or more transgenes or mutations provided herein with a second tobacco variety without the one or more transgenes or mutations to produce one or more progeny tobacco plants; (b) genotyping the one or more progeny tobacco plants for the one or more transgenes or mutations; and (c) selecting a progeny tobacco plant comprising the one or more transgenes or mutations. In another aspect, these methods further comprise backcrossing the selected progeny tobacco plant with the second tobacco variety. In further aspects, these methods further comprise: (d) crossing the selected progeny plant with itself or with the second tobacco variety to produce one or more further progeny tobacco plants; and (c) selecting a further progeny tobacco plant comprising the one or more transgenes or mutations. In one aspect, the second tobacco variety is an elite variety.

In one aspect, the present disclosure provides a method of growing a population of modified tobacco plants disclosed herein, where the method comprises planting a population of tobacco seeds comprising one or more mutations, one or more transgenes, or both as described herein, where the one or more modified tobacco plants or cured leaf of one or more modified tobacco plants comprise a reduced level of one or more TSNAs and further comprises one or more traits selected from the group consisting of an increased level of one or more antioxidants, an increased level of oxygen radical absorbance capacity (ORAC), and a reduced level of nitrite, wherein said reduced or increased level is compared to control tobacco plants or cured leaf of a control tobacco plant of the same variety when grown and cured under comparable conditions.

In one aspect, the present disclosure provides a method of growing a modified tobacco plant described herein comprising planting a modified tobacco seed described herein; and growing the modified tobacco plant from the seed. In an aspect, growing comprises germinating a seed. In another aspect, growing comprises placing a seedling in soil, agar, agar-based media, or a hydroponics system. In another aspect, growing comprises providing a seed or plant with water, light (e.g., artificial light, sunlight), fertilizer, a rooting media, or a combination thereof. In an aspect, growing can take place indoors (e.g., a greenhouse) or outdoors (e.g., a field). In one aspect, growing comprises placing a seed or a plant in a container.

In one aspect, this disclosure provides a method for manufacturing a modified seed, comprising introducing a recombinant DNA construct provided herein into a plant cell; screening a population of plant cells for the recombinant DNA construct; selecting one or more plant cells from the population; generating one or more modified plants from the one or more plant cells; and collecting one or more modified seeds from the one or more modified plants.

As used herein, “locus” is a chromosome region where a polymorphic nucleic acid, trait determinant, gene, or marker is located. The loci of this disclosure comprise one or more polymorphisms in a population; e.g., alternative alleles are present in some individuals. As used herein, “allele” refers to an alternative nucleic acid sequence at a particular locus. The length of an allele can be as small as 1 nucleotide base, but is typically larger. For example, a first allele can occur on one chromosome, while a second allele occurs on a second homologous chromosome, e.g., as occurs for different chromosomes of a heterozygous individual, or between different homozygous or heterozygous individuals in a population. As used herein, a chromosome in a diploid plant is “hemizygous” when only one copy of a locus is present. For example, an inserted transgene is hemizygous when it only inserts into one sister chromosome (i.e., the second sister chromosome does not contain the inserted transgene).

In one aspect, a modified plant, seed, plant component, plant cell, or plant genome is homozygous for a transgene provided herein. In another aspect, a modified plant, seed, plant component, plant cell, or plant genome is heterozygous for a transgene provided herein. In one aspect, a modified plant, seed, plant component, plant cell, or plant genome is hemizygous for a transgene provided herein. In one aspect, a modified plant, seed, plant component, plant cell, or plant genome is homozygous for a mutation provided herein. In another aspect, a modified plant, seed, plant component, plant cell, or plant genome is heterozygous for a mutation provided herein. In one aspect, a modified plant, seed, plant component, plant cell, or plant genome is hemizygous for a mutation provided herein.

As used herein, “introgression” or “introgress” refers to the transmission of a desired allele of a genetic locus from one genetic background to another.

As used herein, “crossed” or “cross” means to produce progeny via fertilization (e.g. cells, seeds or plants) and includes crosses between different plants (sexual) and self-fertilization (selfing).

As used herein, “backcross” and “backcrossing” refer to the process whereby a progeny plant is repeatedly crossed back to one of its parents. In a backcrossing scheme, the “donor” parent refers to the parental plant with the desired gene or locus to be introgressed. The “recipient” parent (used one or more times) or “recurrent” parent (used two or more times) refers to the parental plant into which the gene or locus is being introgressed. The initial cross gives rise to the F₁ generation. The term “BC₁” refers to the second use of the recurrent parent, “BC₂” refers to the third use of the recurrent parent, and so on. In one aspect, a backcross is performed repeatedly, with a progeny individual of each successive backcross generation being itself backcrossed to the same parental genotype.

As used herein, “elite variety” means any variety that has resulted from breeding and selection for superior agronomic performance.

As used herein, “selecting” or “selection” in the context of breeding refer to the act of picking or choosing desired individuals, normally from a population, based on certain pre-determined criteria.

In one aspect, tobacco plants provided herein are hybrid plants. Hybrids can be produced by preventing self-pollination of female parent plants (e.g., seed parents) of a first variety, permitting pollen from male parent plants of a second variety to fertilize the female parent plants, and allowing F₁ hybrid seeds to form on the female plants. Self-pollination of female plants can be prevented by emasculating the flowers at an early stage of flower development. Alternatively, pollen formation can be prevented on the female parent plants using a form of male sterility. For example, male sterility can be produced by male sterility (MS), or transgenic male sterility where a transgene inhibits microsporogenesis and/or pollen formation, or self-incompatibility. Female parent plants containing MS are particularly useful. In aspects in which the female parent plants are MS, pollen may be harvested from male fertile plants and applied manually to the stigmas of MS female parent plants, and the resulting F₁ seed is harvested. Additionally, female sterile plants can also be used to prevent self-fertilization.

Plants can be used to form single-cross tobacco F₁ hybrids. Pollen from a male parent plant is manually transferred to an emasculated female parent plant or a female parent plant that is male sterile to form F₁ seed. Alternatively, three-way crosses can be carried out where a single-cross F₁ hybrid is used as a female parent and is crossed with a different male parent. As another alternative, double-cross hybrids can be created where the F₁ progeny of two different single-crosses are themselves crossed. Self-incompatibility can be used to particular advantage to prevent self-pollination of female parents when forming a double-cross hybrid.

In one aspect, a tobacco variety provided herein is male sterile. In another aspect, a tobacco variety provided herein is cytoplasmic male sterile (CMS). Male sterile tobacco plants may be produced by any method known in the art. Methods of producing male sterile tobacco are described in Wernsman, E. A., and Rufty, R. C. 1987. Chapter Seventeen. Tobacco. Pages 669-698 In: Cultivar Development. Crop Species. W. H. Fchr (ed.), MacMillan Publishing Go., Inc., New York, N.Y. 761 pp. In another aspect, a tobacco variety provided herein is female sterile. As a non-limiting example, female sterile plants can be made by mutating the STIGI gene. See, for example, Goldman et al. 1994, EMBO Journal 13:2976-2984.

As used herein, the term “sequence identity” or “identity” in the context of two polynucleotide or polypeptide sequences makes reference to the residues in the two sequences that are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity.”

The use of the term “polynucleotide” is not intended to limit the present disclosure to polynucleotides comprising DNA. Those of ordinary skill in the art will recognize that polynucleotides and nucleic acid molecules can comprise ribonucleotides and combinations of ribonucleotides and deoxyribonucleotides. Such deoxyribonucleotides and ribonucleotides include both naturally occurring molecules and synthetic analogues. The polynucleotides of the present disclosure also encompass all forms of sequences including, but not limited to, single-stranded forms, double-stranded forms, hairpins, stem-and-loop structures, and the like.

As used herein, the term “polypeptide” refers to a chain of at least two covalently linked amino acids.

Nucleic acid molecules, polypeptides, or proteins provided herein can be isolated or substantially purified. An “isolated” or “purified” nucleic acid molecule, polypeptide, protein, or biologically active portion thereof, is substantially or essentially free from components that normally accompany or interact with the polynucleotide or protein as found in its naturally occurring environment. For example, an isolated or purified polynucleotide or protein is substantially free of other cellular material, or culture medium when produced by recombinant techniques, or substantially free of chemical precursors or other chemicals when chemically synthesized. In another aspect, an isolated polypeptide provided herein is substantially free of cellular material in preparations having less than about 30%, less than about 20%, less than about 10%, less than about 5%, or less than about 1% (by dry weight) of chemical precursors or non-protein-of-interest chemicals. Fragments of the disclosed polynucleotides and polypeptides encoded thereby are also encompassed by the present invention. Fragments of a polynucleotide may encode polypeptide fragments that retain the biological activity of the native polypeptide. Alternatively, fragments of a polynucleotide that are useful as hybridization probes or PCR primers using methods known in the art generally do not encode fragment polypeptides retaining biological activity. Fragments of a polynucleotide provided herein can range from at least about 20 nucleotides, about 50 nucleotides, about 70 nucleotides, about 100 nucleotides, about 150 nucleotides, about 200 nucleotides, about 250 nucleotides, about 300 nucleotides, and up to the full-length polynucleotide encoding the polypeptides of the invention, depending on the desired outcome.

Nucleic acids can be isolated using techniques routine in the art. For example, nucleic acids can be isolated using any method including, without limitation, recombinant nucleic acid technology, and/or the polymerase chain reaction (PCR). General PCR techniques are described, for example in PCR Primer: A Laboratory Manual, Dieffenbach & Dveksler, Eds., Cold Spring Harbor Laboratory Press, 1995. Recombinant nucleic acid techniques include, for example, restriction enzyme digestion and ligation, which can be used to isolate a nucleic acid. Isolated nucleic acids also can be chemically synthesized, either as a single nucleic acid molecule or as a series of oligonucleotides. Polypeptides can be purified from natural sources (e.g., a biological sample) by known methods such as DEAE ion exchange, gel filtration, and hydroxyapatite chromatography. A polypeptide also can be purified, for example, by expressing a nucleic acid in an expression vector. In addition, a purified polypeptide can be obtained by chemical synthesis. The extent of purity of a polypeptide can be measured using any appropriate method, e.g., column chromatography, polyacrylamide gel electrophoresis, or HPLC analysis.

In one aspect, this disclosure provides methods of detecting in plant cells one or more recombinant nucleic acids and polypeptides described here. Without being limiting, nucleic acids also can be detected using hybridization. Hybridization between nucleic acids is discussed in detail in Sambrook et al. (1989, Molecular Cloning: A Laboratory Manual, 2nd Ed., Cold Spring Harbor Laboratory Press, Cold Spring Harbor, NY).

Polypeptides can be detected using antibodies. Techniques for detecting polypeptides using antibodies include enzyme linked immunosorbent assays (ELISAs), Western blots, immunoprecipitations and immunofluorescence. An antibody provided herein can be a polyclonal antibody or a monoclonal antibody. An antibody having specific binding affinity for a polypeptide provided herein can be generated using methods well known in the art. An antibody provided herein can be attached to a solid support such as a microtiter plate using methods known in the art.

Detection (e.g., of an amplification product, of a hybridization complex, of a polypeptide) can be accomplished using detectable labels. The term “label” is intended to encompass the use of direct labels as well as indirect labels. Detectable labels include enzymes, prosthetic groups, fluorescent materials, luminescent materials, bioluminescent materials, and radioactive materials.

In one aspect, the present disclosure also provides a method of reducing the level of one or more TSNAs in cured leaf from a tobacco plant, the method comprising increasing the level of one or more antioxidants in the tobacco plant by expressing a biosynthetic enzyme, a regulatory transcription factor, a transporter, a catabolic enzyme, or a combination thereof, for the one or more antioxidants. In another aspect, a method comprises expressing a gene promoting the production or accumulation of one or more antioxidants are selected from the group consisting of anthocyanidin, flavanone, flavanol, flavone, flavonol, isoflavone, hydroxybenzoic acid, hydroxycinnamic acid, ellagitannin, stibene, lignan, carotenoids, and glycyrrhzin. In a further aspect, a method comprises expressing a gene promoting the production or accumulation of one or more antioxidants are selected from the group consisting of Delphnidin, Cyanidin, Procyanidin, Prodelphinidin, Hesperetin, Naringenin, Catechin, Epicatechin, Apigenin, Luteonin, Quercetin, Myricetin, Rutin, Genistein, Daidzein, Gallic acid, Vanillic acid, Protocatechuic acid, Ferunic acid, Cinnamic acid, Coumeric acid, Chlorogenic acid, Coffeic acid, ferulic acid, Sanguiin, Resveratrol, Sesamin, Caretonoids, and Vitamin C. In one aspect, a method does not substantially reduce the level of total alkaloids in the tobacco plant. In another aspect, a method does not substantially reduce the level of nicotine in the tobacco plant.

In one aspect, the present disclosure provides a method for reducing the level of one or more TSNAs in cured tobacco leaf or a tobacco product made therefrom, the method comprising increasing the level of one or more antioxidants in a tobacco plant via a transgene encoding or directly modulating an antioxidant biosynthetic enzyme, a regulatory transcription factor of an antioxidant, an antioxidant transporter, an antioxidant metabolic enzyme, or a combination thereof; and reducing the level of one or more TSNAs in cured tobacco leaf from the tobacco plant or a tobacco product made from the cured tobacco leaf. In another aspect of a method described herein, the level of one or more TSNAs reduces by at least 50%, by at least 45%, by at least 40%, by at least 35%, by at least 30%, by at least 25%, by at least 20%, by at least 15%, by at least 10%, or by at least 5%, compared to cured leaf from a control tobacco plant not comprising a transgene. In a further aspect of a method described herein, cured leaf of the modified tobacco plant produces or comprises less than 2, less than 1.9, less than 1.8, less than 1.7, less than 1.6, less than 1.5, less than 1.4, less than 1.3, less than 1.2, less than 1.1, less than 1.0, less than 0.9, less than 0.8, less than 0.7, less than 0.6, less than 0.5, less than 0.4, less than 0.3, less than 0.2, less than 0.15, less than 0.1, or less than 0.05 ppm total TSNAs. In a further aspect of a method described herein, cured leaf of the modified tobacco plant comprises between 2 and 0.05, between 1.9 and 0.05, between 1.8 and 0.05, between 1.7 and 0.05, between 1.6 and 0.05, between 1.5 and 0.05, between 1.4 and 0.05, between 1.3 and 0.05, between 1.2 and 0.05, between 1.1 and 0.05, between 1.0 and 0.05, between 0.9 and 0.05, between 0.8 and 0.05, between 0.7 and 0.05, between 0.6 and 0.05, between 0.5 and 0.05, between 0.4 and 0.05, between 0.3 and 0.05, between 0.2 and 0.05, between 0.15 and 0.05, or between 0.1 and 0.05 ppm total TSNAs. In a further aspect of a method described herein, cured leaf of the modified tobacco plant comprises between 2 and 0.05, between 1.8 and 0.1, between 1.5 and 0.15, between 1.2 and 0.2, between 1.0 and 0.3, between 0.8 and 0.4, or between 0.6 and 0.5 ppm total TSNAs. In a further aspect of a method described herein, one or more TSNAs are selected from the group consisting of N′-nitrosonornicotine (NNN), 4-methylnitrosoamino-1-(3-pyridyl)-1-butanone (NNK), N′-nitrosoanatabine (NAT), N′-nitrosoanabasine (NAB), and any combination thereof. In a further aspect of a method described herein, the TSNA reduction comprises a reduction of NNK. In a further aspect of a method described herein, the TSNA reduction consists of a reduction of NNK. In a further aspect of a method described herein, NNK is reduced below 0.08 parts per million, below 0.07 parts per million, below 0.06 parts per million, or below 0.05 parts per million, as measured in freeze-dried cured leaf samples using liquid chromatography with tandem mass spectrometry.

In another aspect of a method described herein, the tobacco plant comprises reduced nicotine demethylase activity compared to a control plant. In a further aspect of a method described herein, the tobacco plant comprises at least one mutation in a nicotine demethylase gene selected from the group consisting of CYP82E4, CYP82E5, CYP82E10, and a combination thereof. In another aspect of a method described herein, a method reduces nitrite levels in cured tobacco leaf comprising the transgene. In another aspect of a method described herein, a method increases the oxygen radical absorbance capacity level in cured tobacco leaf comprising the transgene. In another aspect of a method described herein, the one or more antioxidants that are increased in cured tobacco leaf comprising the transgene are selected from the group consisting of anthocyanidin, flavanone, flavanol, flavone, flavonol, isoflavone, hydroxybenzoic acid, hydroxycinnamic acid, ellagitannin, stibene, lignan, carotenoids, and glycyrrhzin. In a further aspect of a method described herein, the one or more antioxidants that are increased in cured tobacco leaf comprising the transgene are selected from the group consisting of Delphnidin, Cyanidin, Procyanidin, Prodelphinidin, Hesperetin, Naringenin, Catechin, Epicatechin, Apigenin, Luteonin, Quercetin, Myricetin, Rutin, Genistein, Daidzein, Gallic acid, Vanillic acid, Protocatechuic acid, Ferunic acid, Cinnamic acid, Coumeric acid, Chlorogenic acid, Coffeic acid, ferulic acid, Sanguiin, Resveratrol, Sesamin, Caretonoids, and Vitamin C In a further aspect of a method described herein, a method does not substantially reduce the level of total alkaloids in a tobacco plant. In a further aspect of a method described herein, a method does not substantially reduce the level of nicotine in a tobacco plant. In an aspect of a method described herein, a transgene encodes or directly modulates a biosynthetic enzyme, a regulatory transcription factor, a transporter, a metabolic enzyme, or a combination thereof, for one or more antioxidants selected from the group consisting of anthocyanidin, flavanone, flavanol, flavone, flavonol, isoflavone, hydroxybenzoic acid, hydroxycinnamic acid, ellagitannin, stibene, lignan, carotenoids, and glycyrrhzin. In a further aspect of a method described herein, a transgene encodes or directly modulates a biosynthetic enzyme, a regulatory transcription factor, a transporter, a metabolic enzyme, or a combination thereof, for one or more antioxidants selected from the group consisting of Delphnidin, Cyanidin, Procyanidin, Prodelphinidin, Hesperetin, Naringenin, Catechin, Epicatechin, Apigenin, Luteonin, Quercetin, Myricetin, Rutin, Genistein, Daidzein, Gallic acid, Vanillic acid, Protocatechuic acid, Ferunic acid, Cinnamic acid, Coumeric acid, Chlorogenic acid, Coffeic acid, ferulic acid, Sanguiin, Resveratrol, Sesamin, Caretonoids, and Vitamin C In another aspect of a method described herein, the transgene encodes a protein comprising a sequence having at least 50%, 55%, 60%, 65%, 70%, 75%, 80%, 85%, 90%, 95%, 97%, 98%, 99%, or 100% identity to a sequence selected from the group consisting of SEQ ID No. 1 to 23 and 47 to 52.

In one aspect, the present disclosure provides a method for reducing the level of one or more TSNAs in cured tobacco leaf or a tobacco product made therefrom, a method comprising increasing the level of one or more antioxidants in a tobacco plant via a genetic modification in an endogenous gene, wherein the endogenous gene encodes an antioxidant biosynthetic enzyme, a regulatory transcription factor of an antioxidant, an antioxidant transporter, an antioxidant metabolic enzyme, or a combination thereof; and reducing the level of one or more TSNAs in cured tobacco leaf from the tobacco plant or a tobacco product made from the cured tobacco leaf. In another aspect of a method described herein, the level of one or more TSNAs is reduced by at least 50%, by at least 45%, by at least 40%, by at least 35%, by at least 30%, by at least 25%, by at least 20%, by at least 15%, by at least 10%, or by at least 5%, compared to cured leaf from a control tobacco plant not comprising a transgene. In a further aspect of a method described herein, one or more TSNAs are selected from the group consisting of N′-nitrosonornicotine (NNN), 4-methylnitrosoamino-1-(3-pyridyl)-1-butanone (NNK), N′-nitrosoanatabine (NAT) N′-nitrosoanabasine (NAB), and any combination thereof. In further aspect of a method described herein, the TSNA reduction comprises a reduction of NNK. In a further aspect of a method described herein, the TSNA reduction consists of a reduction of NNK. In a further aspect of a method described herein, NNK is reduced below 0.08 parts per million as measured in freeze-dried cured leaf samples using liquid chromatography with tandem mass spectrometry.

In another aspect of a method described herein, a tobacco plant comprises reduced nicotine demethylase activity compared to a control plant. In a further aspect of a method described herein, a tobacco plant comprises at least one mutation in a nicotine demethylase gene selected from the group consisting of CYP82E4, CYP82E5, CYP82E10, and a combination thereof. In another aspect of a method described herein, a method reduces nitrite levels in cured tobacco leaf comprising a transgene. In another aspect of a method described herein, a method increases the oxygen radical absorbance capacity level in cured tobacco leaf comprising a transgene. In another aspect of a method described herein, a method increases the oxygen radical absorbance capacity level in cured tobacco leaf comprising a transgene. In another aspect of a method described herein, one or more increased antioxidants are tobacco native antioxidants. In another aspect of a method described herein, the one or more antioxidants that are increased in cured tobacco leaf comprising a transgene are selected from the group consisting of anthocyanidin, flavanone, flavanol, flavone, flavonol, isoflavone, hydroxybenzoic acid, hydroxycinnamic acid, ellagitannin, stibene, lignan, carotenoids, and glycyrrhzin. In a further aspect of the method, the one or more antioxidants that are increased in cured tobacco leaf comprising the transgene are selected from the group consisting of Delphnidin, Cyanidin, Procyanidin, Prodelphinidin, Hesperetin, Naringenin, Catechin, Epicatechin, Apigenin, Luteonin, Quercetin, Myricetin, Rutin, Genistein, Daidzein, Gallic acid, Vanillic acid, Protocatechuic acid, Ferunic acid, Cinnamic acid, Coumeric acid, Chlorogenic acid, Coffeic acid, ferulic acid, Sanguiin, Resveratrol, Sesamin, Caretonoids, and Vitamin C.

In a further aspect of a method described herein, a method does not substantially reduce the level of total alkaloids in a tobacco plant. In a further aspect of a method described herein, a method does not substantially reduce the level of nicotine in a tobacco plant. In another aspect of a method described herein, an endogenous gene encodes a protein comprising a sequence having at least 50%, 55%, 60%, 65%, 70%, 75%, 80%, 85%, 90%, 95%, 97%, 98%, 99%, or 100% identity to a sequence selected from the group consisting of SEQ ID No. 1 to 23 and 47 to 52.

In one aspect, the present disclosure provides a method for manufacturing a tobacco product, the method comprising obtaining cured tobacco leaf comprising a transgene or comprising a genetic modification in an endogenous gene, and further comprising an increased level of one or more antioxidants compared to cured tobacco leaf control lacking a transgene or a genetic modification, wherein an endogenous gene encodes an antioxidant biosynthetic enzyme, a regulatory transcription factor of an antioxidant, an antioxidant transporter, an antioxidant metabolic enzyme, or a combination thereof, wherein a transgene encodes or directly modulates an antioxidant biosynthetic enzyme, a regulatory transcription factor of an antioxidant, an antioxidant transporter, an antioxidant metabolic enzyme, or a combination thereof; and producing a tobacco product from cured tobacco leaf, wherein a tobacco product comprises a reduced level of one or more TSNAs relative to a control tobacco product prepared from a control cured tobacco leaf. In another aspect of a method described herein, cured tobacco leaf comprises a transgene encoding or directly modulating an antioxidant biosynthetic enzyme, a regulatory transcription factor of an antioxidant, an antioxidant transporter, an antioxidant metabolic enzyme, or a combination thereof. In another aspect of a method described herein, cured tobacco leaf comprises a genetic modification in an antioxidant biosynthetic enzyme, a regulatory transcription factor of an antioxidant, an antioxidant transporter, an antioxidant metabolic enzyme, or a combination thereof.

In another aspect of a method described herein, the level of one or more TSNAs is reduced by at least 50%, by at least 45%, by at least 40%, by at least 35%, by at least 30%, by at least 25%, by at least 20%, by at least 15%, by at least 10%, or by at least 5%, compared to cured leaf from a control tobacco plant not comprising a transgene. In another aspect of a method described herein, the level of one or more TSNAs is reduced by at least 50%, by at least 45%, by at least 40%, by at least 35%, by at least 30%, by at least 25%, by at least 20%, by at least 15%, by at least 10%, or by at least 5%, compared to cured leaf from a control tobacco plant not comprising a genetic modification in an endogenous gene. In another aspect of a method described herein, cured tobacco leaf comprises a reduced nitrite level compared to a control plant without a transgene. In another aspect of a method described herein, the cured tobacco leaf comprises a reduced nitrite level compared to a control plant without a genetic modification in an endogenous gene.

In one aspect, the present disclosure provides a method for preparing cured tobacco leaf, the method comprising growing a tobacco plant comprising a transgene or a genetic modification in an endogenous gene, and further comprising an increased level of one or more antioxidants compared to a control cured tobacco leaf lacking a transgene or a genetic modification, wherein an endogenous gene encodes an antioxidant biosynthetic enzyme, a regulatory transcription factor of an antioxidant, an antioxidant transporter, an antioxidant metabolic enzyme, or a combination thereof, wherein a transgene encodes or directly modulates an antioxidant biosynthetic enzyme, a regulatory transcription factor of an antioxidant, an antioxidant transporter, an antioxidant metabolic enzyme, or a combination thereof; and preparing cured leaf from a tobacco plant, wherein cured leaf comprises a reduced level of one or more TSNAs relative to a control cured leaf from a control tobacco plant not comprising a transgene or a genetic modification. In another aspect of a method described herein, cured tobacco leaf comprises a transgene encoding or directly modulating an antioxidant biosynthetic enzyme, a regulatory transcription factor of an antioxidant, an antioxidant transporter, an antioxidant metabolic enzyme, or a combination thereof. In another aspect of a method described herein, cured tobacco leaf comprises a genetic modification in an antioxidant biosynthetic enzyme, a regulatory transcription factor of an antioxidant, an antioxidant transporter, an antioxidant metabolic enzyme, or a combination thereof.

In another aspect of a method described herein, the level of one or more TSNAs is reduced by at least 50%, by at least 45%, by at least 40%, by at least 35%, by at least 30%, by at least 25%, by at least 20%, by at least 15%, by at least 10%, or by at least 5%, compared to cured leaf from a control tobacco plant not comprising a transgene. In another aspect of a method described herein, the level of one or more TSNAs is reduced by at least 50%, by at least 45%, by at least 40%, by at least 35%, by at least 30%, by at least 25%, by at least 20%, by at least 15%, by at least 10%, or by at least 5%, compared to cured leaf from a control tobacco plant not comprising a genetic modification in an endogenous gene. In another aspect of a method described herein, cured tobacco leaf comprises a reduced nitrite level compared to a control plant without a transgene. In another aspect of a method described herein, cured tobacco leaf comprises a reduced nitrite level compared to a control plant without a genetic modification in an endogenous gene.

In one aspect, the present disclosure provides cured leaf of a modified tobacco plant, wherein cured leaf comprises a reduced level of one or more tobacco-specific nitrosamines (TSNAs) and further comprises an increased level of one or more antioxidants and a reduced nitrite level, wherein reduced and increased levels are compared to a control cured leaf of an unmodified tobacco plant of the same variety when grown and cured under comparable conditions, wherein a modification comprises a transgene or a genetic modification in an endogenous gene, wherein a transgene or an endogenous gene encodes an antioxidant biosynthetic enzyme, a regulatory transcription factor of an antioxidant, an antioxidant transporter, an antioxidant metabolic enzyme, or a combination thereof; wherein a modified tobacco plant does not comprise a transgene overexpressing an Arabidopsis PAP1 protein. In a further aspect of a method described herein, cured leaf of the modified tobacco plant produces or comprises less than 2, less than 1.9, less than 1.8, less than 1.7, less than 1.6, less than 1.5, less than 1.4, less than 1.3, less than 1.2, less than 1.1, less than 1.0, less than 0.9, less than 0.8, less than 0.7, less than 0.6, less than 0.5, less than 0.4, less than 0.3, less than 0.2, less than 0.15, less than 0.1, or less than 0.05 ppm total TSNAs. In a further aspect of a method described herein, cured leaf of the modified tobacco plant comprises between 2 and 0.05, between 1.9 and 0.05, between 1.8 and 0.05, between 1.7 and 0.05, between 1.6 and 0.05, between 1.5 and 0.05, between 1.4 and 0.05, between 1.3 and 0.05, between 1.2 and 0.05, between 1.1 and 0.05, between 1.0 and 0.05, between 0.9 and 0.05, between 0.8 and 0.05, between 0.7 and 0.05, between 0.6 and 0.05, between 0.5 and 0.05, between 0.4 and 0.05, between 0.3 and 0.05, between 0.2 and 0.05, between 0.15 and 0.05, or between 0.1 and 0.05 ppm total TSNAs. In a further aspect of a method described herein, cured leaf of the modified tobacco plant comprises between 2 and 0.05, between 1.8 and 0.1, between 1.5 and 0.15, between 1.2 and 0.2, between 1.0 and 0.3, between 0.8 and 0.4, or between 0.6 and 0.5 ppm total TSNAs. In a further aspect of a method described herein, one or more TSNAs are selected from the group consisting of N′-nitrosonornicotine (NNN), 4-methylnitrosoamino-1-(3-pyridyl)-1-butanone (NNK), N′-nitrosoanatabine (NAT) N′-nitrosoanabasine (NAB), and any combination thereof. In a further aspect of a method described herein, the TSNA reduction comprises a reduction of NNK. In a further aspect of a method described herein, the TSNA reduction consists of a reduction of NNK. In a further aspect of a method described herein, NNK is reduced below 0.08 parts per million, below 0.07 parts per million, below 0.06 parts per million, or below 0.05 parts per million, as measured in freeze-dried cured leaf samples using liquid chromatography with tandem mass spectrometry. In another aspect of a method described herein, a tobacco plant comprises reduced nicotine demethylase activity compared to a control plant. In a further aspect of a method described herein, a tobacco plant comprises at least one mutation in a nicotine demethylase gene selected from the group consisting of CYP82E4, CYP82E5, CYP82E10, and a combination thereof. In a further aspect of a method described herein, a method provides a tobacco product comprising cured leaf of a modified tobacco plant.

Having now generally described the disclosure, the same will be more readily understood through reference to the following examples that are provided by way of illustration, and are not intended to be limiting of the present disclosure, unless specified.

The following are exemplary embodiments of the present disclosure.
Embodiment 1. Cured leaf of a modified tobacco plant, wherein said cured leaf comprises a reduced level of one or more tobacco-specific nitrosamines (TSNAs) and further comprises an increased level of one or more antioxidants, wherein said reduced and increased levels are compared to a control cured leaf of an unmodified tobacco plant of the same variety when grown and cured under comparable conditions.
Embodiment 2. A tobacco product comprising the cured leaf of embodiment 1.
Embodiment 3. A tobacco product made from the cured leaf of any one of embodiments 1 or 2.
Embodiment 4. The cured leaf of any one of embodiments 1 to 3, wherein said one or more antioxidants are endogenous antioxidants.
Embodiment 5. The cured leaf of any one of embodiments 1 to 4, wherein said one or more antioxidants are not exogenous antioxidants administered, added, or introduced to said cured leaf or modified tobacco plant.
Embodiment 6. The cured leaf of any one of embodiments 1 to 5, wherein said one or more antioxidants are produced by said modified tobacco plant.
Embodiment 7. The cured leaf of any one of embodiments 1 to 6, wherein said one or more antioxidants are produced in vivo in said cured leaf.
Embodiment 8. The cured leaf of any one of embodiments 1 to 7, wherein said cured leaf comprises an increased level of oxygen radical absorbance capacity (ORAC) compared to said control cured leaf.
Embodiment 9. The cured leaf of any one of embodiments 1 to 8, wherein said modified tobacco plant comprises in a green leaf an increased level of oxygen radical absorbance capacity (ORAC) compared to said unmodified tobacco plant of the same variety when grown under comparable conditions.
Embodiment 10. The cured leaf of any one of embodiments 1 and 4 to 9, wherein said reduced or increased level is within about 10%, within about 20%, within about 30%, within about 40%, within about 50%, within about 60%, within about 70%, within about 80%, within about 90%, within about 92%, within about 94%, within about 95%, within about 96%, within about 97%, within about 98%, or within about 99% lower or higher than the level in said control cured leaf.
Embodiment 11. The cured leaf of any one of embodiments 1 and 4 to 10, wherein said reduced or increased level is within about 1 fold, within about 2 folds, within about 3 folds, within about 4 folds, within about 5 folds, within about 6 folds, within about 7 folds, within about 8 folds, within about 9 folds, within about 10 folds, within about 15 folds, within about 20 folds, within about 25 folds, or within about 30 folds lower or higher than the level in said control cured leaf.
Embodiment 12. The cured leaf of any one of embodiments 1 and 4 to 11, wherein said reduced or increased level is at least about 10%, at least about 20%, at least about 30%, at least about 40%, at least about 50%, at least about 60%, at least about 70%, at least about 80%, at least about 90%, at least about 92%, at least about 94%, at least about 95%, at least about 96%, at least about 97%, at least about 98%, or at least about 99% lower or higher than the level in said control cured leaf.
Embodiment 13. The cured leaf of any one of embodiments 1 and 4 to 12, wherein said reduced or increased level is at least about 1 fold, at least about 2 folds, at least about 3 folds, at least about 4 folds, at least about 5 folds, at least about 6 folds, at least about 7 folds, at least about 8 folds, at least about 9 folds, at least about 10 folds, at least about 15 folds, at least about 20 folds, at least about 25 folds, or at least about 30 folds lower or higher than the level in said control cured leaf.
Embodiment 14. The cured leaf of any one of embodiments 1 and 4 to 13, wherein said reduced or increased level is about 10%, about 20%, about 30%, about 40%, about 50%, about 60%, about 70%, about 80%, about 90%, about 92%, about 94%, about 95%, about 96%, about 97%, about 98%, or about 99% lower or higher than the level in said control cured leaf.
Embodiment 15. The cured leaf of any one of embodiments 1 and 4 to 14, wherein said reduced or increased level is about 1 fold, about 2 folds, about 3 folds, about 4 folds, about 5 folds, about 6 folds, about 7 folds, about 8 folds, about 9 folds, about 10 folds, about 15 folds, about 20 folds, about 25 folds, or about 30 folds lower or higher than the level in said control cured leaf.
Embodiment 16. The cured leaf of any one of embodiments 1 and 4 to 15, wherein said reduced or increased level is about 1-2 folds, about 2-3 folds, about 3-4 folds, about 4-5 folds, about 5-6 folds, about 6-7 folds, about 7-8 folds, about 8-9 folds, about 9-10 folds, about 10-15 folds, about 15-20 folds, about 20-25 folds, about 25-30 folds, or about 30-50 folds lower or higher than the level in said control cured leaf.
Embodiment 17. The cured leaf of any one of embodiments 1 and 4 to 16, wherein said reduced or increased level is about 1-10 folds, about 2-10 folds, about 3-10 folds, about 4-10 folds, about 5-10 folds, about 6-10 folds, about 7-10 folds, about 8-10 folds, about 9-10 folds, about 10-50 folds, about 15-50 folds, about 20-50 folds, about 25-50 folds, or about 30-50 folds lower or higher than the level in said control cured leaf.
Embodiment 18. The cured leaf of any one of embodiments 1 to 9, wherein said increased level of one or more antioxidants is from the modification in said modified tobacco plant.
Embodiment 19. The cured leaf of embodiment 18, wherein said modification in said modified tobacco plant comprises a tobacco genome mutation or a transgene.
Embodiment 20. The cured leaf of any one of embodiments 18 or 19, wherein said modification in said modified tobacco plant comprises a tobacco genome mutation in a gene encoding a biosynthetic enzyme, a regulatory transcription factor, a transporter, a catabolic enzyme, or a combination thereof, for one or more antioxidants selected from the group consisting of anthocyanidin, flavanone, flavanol, flavone, flavonol, isoflavone, hydroxybenzoic acid, hydroxycinnamic acid, ellagitannin, stibene, lignan, carotenoids, and glycyrrhzin.
Embodiment 21. The cured leaf of any one of embodiments 18 to 20, wherein said modification in said modified tobacco plant comprises a tobacco genome mutation in a gene encoding a biosynthetic enzyme, a regulatory transcription factor, a transporter, a catabolic enzyme, or a combination thereof, for one or more antioxidants selected from the group consisting of Delphnidin, Cyanidin, Procyanidin, Prodelphinidin, Hesperetin, Naringenin, Catechin, Epicatechin, Apigenin, Luteonin, Quercetin, Myricetin, Rutin, Genistein, Daidzein, Gallic acid, Vanillic acid, Protocatechuic acid, Ferunic acid, Cinnamic acid, Coumeric acid, Chlorogenic acid, Coffeic acid, ferulic acid, Sanguiin, Resveratrol, Sesamin, Caretonoids, and Vitamin C.
Embodiment 22. The cured leaf of any one of embodiments 18 to 21, wherein said modification in said modified tobacco plant comprises a tobacco genome mutation in a gene encoding a protein comprising a sequence having at least 50%, 55%, 60%, 65%, 70%, 75%, 80%, 85%, 90%, 95%, 97%, 98%, 99%, or 100% identity to a sequence selected from the group consisting of SEQ ID No. 1 to 23 and 47 to 52.
Embodiment 23. The cured leaf of any one of embodiments 18 to 22, wherein said modification in said modified tobacco plant comprises a transgene encoding or directly modulating a biosynthetic enzyme, a regulatory transcription factor, a transporter, a catabolic enzyme, or a combination thereof, for one or more antioxidants selected from the group consisting of anthocyanidin, flavanone, flavanol, flavone, flavonol, isoflavone, hydroxybenzoic acid, hydroxycinnamic acid, ellagitannin, stibene, lignan, carotenoids, and glycyrrhzin.
Embodiment 24. The cured leaf of any one of embodiments 18 to 23, wherein said modification in said modified tobacco plant comprises a transgene encoding or directly modulating a biosynthetic enzyme, a regulatory transcription factor, a transporter, a catabolic enzyme, or a combination thereof, for one or more antioxidants selected from the group consisting of Delphnidin, Cyanidin, Procyanidin, Prodelphinidin, Hesperetin, Naringenin, Catechin, Epicatechin, Apigenin, Luteonin, Quercetin, Myricetin, Rutin, Genistein, Daidzein, Gallic acid, Vanillic acid, Protocatechuic acid, Ferunic acid, Cinnamic acid, Coumeric acid, Chlorogenic acid, Coffeic acid, ferulic acid, Sanguiin, Resveratrol, Sesamin, Caretonoids, and Vitamin C.
Embodiment 25. The cured leaf of any one of embodiments 18 to 24, wherein said modification in said modified tobacco plant comprises a transgene encoding a protein comprising a sequence having at least 50%, 55%, 60%, 65%, 70%, 75%, 80%, 85%, 90%, 95%, 97%, 98%, 99%, or 100% identity to a sequence selected from the group consisting of SEQ ID No. 1 to 23 and 47 to 52.
Embodiment 26. The cured leaf of any one of embodiments 18 to 25, wherein said modification in said modified tobacco plant comprises a transgene that targets a gene comprising a sequence having at least 50%, 55%, 60%, 65%, 70%, 75%, 80%, 85%, 90%, 95%, 97%, 98%, 99%, or 100% identity to a sequence selected from the group consisting of SEQ ID No. 24 to 46 and 53 to 58.
Embodiment 27. The cured leaf of any one of embodiments 1 to 26, wherein said cured leaf comprises a reduced level of nitrite compared to said control cured leaf.
Embodiment 28. The cured leaf of any one of embodiments 1 to 27, wherein said cured leaf is flue-cured tobacco, air-cured burley tobacco, air-cured dark tobacco, fire-cured dark tobacco, or oriental tobacco.
Embodiment 29. Cured leaf from a modified tobacco plant comprising a reduced level of one or more tobacco-specific nitrosamines (TSNAs) and further comprising an increased level of one or more antioxidants, wherein said reduced and increased levels are compared to a control tobacco plant of the same variety when grown and cured under comparable conditions.
Embodiment 30. The cured leaf from a modified tobacco plant of embodiment 29, wherein said cured leaf from a modified tobacco plant comprises an increased level of oxygen radical absorbance capacity (ORAC) compared to said control tobacco plant when grown and cured undergrown and cured under comparable conditions.
Embodiment 31. The cured leaf from a modified tobacco plant of any one of embodiments 29 or 30, wherein said cured leaf from a modified tobacco plant comprises a reduced level of nitrite compared to said control tobacco plant when grown and cured under comparable conditions.
Embodiment 32. The cured leaf from a modified tobacco plant of any one of embodiments 29 to 31, wherein said cured leaf from a modified tobacco plant comprises a reduced level of total TSNAs compared to said control tobacco plant when grown and cured under comparable conditions.
Embodiment 33. The cured leaf from a modified tobacco plant of any one of embodiments 29 to 32, wherein said one or more antioxidants are tobacco endogenous antioxidants.
Embodiment 34. Cured leaf from a modified tobacco plant comprising a reduced level of one or more tobacco-specific nitrosamines (TSNAs) and further comprising a reduced level of nitrite, wherein said reduced levels are compared to cured leaf from a control tobacco plant of the same variety when grown and cured under comparable conditions.
Embodiment 35. The cured leaf from a modified tobacco plant of embodiment 34, wherein said cured leaf from a modified tobacco plant comprises an increased level of oxygen radical absorbance capacity (ORAC) compared to said control tobacco plant when grown and cured under comparable conditions.
Embodiment 36. Cured leaf from a modified tobacco plant comprising a reduced level of one or more tobacco-specific nitrosamines (TSNAs) and further comprising an increased level of oxygen radical absorbance capacity (ORAC), and wherein said reduced and increased levels are compared to cured leaf from a control tobacco plant of the same variety when grown and cured under comparable conditions.
Embodiment 37. The cured leaf from a modified tobacco plant of any one of embodiments 30, 35, or 36, wherein said increased level of oxygen radical absorbance capacity (ORAC) is based on a green leaf sample or cured leaf sample.
Embodiment 38. The cured leaf from a modified tobacco plant of any one of embodiments 29 to 37, wherein said one or more tobacco-specific nitrosamines (TSNAs) are selected from the group consisting of N′-nitrosonornicotine (NNN), 4-methylnitrosoamino-1-(3-pyridyl)-1-butanone (NNK), N′-nitrosoanatabine (NAT) N′-nitrosoanabasine (NAB), and any combination thereof.
Embodiment 39. The cured leaf from a modified tobacco plant of any one of embodiments 29 to 38, wherein said increased level of said one or more TSNAs is based on cured leaf sample.
Embodiment 40. The cured leaf from a modified tobacco plant of any one of embodiments 29 to 39, wherein the level of said one or more TSNAs is measured based on a freeze-dried cured leaf sample using liquid chromatograph with tandem mass spectrometry (LC/MS/MS).
Embodiment 41. The cured leaf from a modified tobacco plant of any one of embodiments 29 to 33, wherein said one or more antioxidants are selected from the group consisting of anthocyanidin, flavanone, flavanol, flavone, flavonol, isoflavone, hydroxybenzoic acid, hydroxycinnamic acid, ellagitannin, stibene, lignan, carotenoids, and glycyrrhzin.
Embodiment 42. The cured leaf from a modified tobacco plant of any one of embodiments 29 to 33, wherein said one or more antioxidants are selected from the group consisting of Delphnidin, Cyanidin, Procyanidin, Prodelphinidin, Hesperetin, Naringenin, Catechin, Epicatechin, Apigenin, Luteonin, Quercetin, Myricetin, Rutin, Genistein, Daidzein, Gallic acid, Vanillic acid, Protocatechuic acid, Ferunic acid, Cinnamic acid, Coumeric acid, Chlorogenic acid, Coffeic acid, ferulic acid, Sanguiin, Resveratrol, Sesamin, Caretonoids, and Vitamin C.
Embodiment 43. The cured leaf from a modified tobacco plant of any one of embodiments 29 to 42, wherein said modified tobacco plant comprises an antioxidant that is undetectable in said cured leaf from a control plant.
Embodiment 44. The cured leaf of a modified tobacco plant of any one of embodiments 29 to 43, wherein said modified tobacco plant comprises an antioxidant that does not exist in said cured leaf from a control plant.
Embodiment 45. The cured leaf from a modified tobacco plant of any one of embodiments 29 to 44, wherein said cured leaf from a modified tobacco plant comprises one or more mutations capable of providing said reduced level of one or more TSNAs.
Embodiment 46. The cured leaf from a modified tobacco plant of embodiment 45, wherein said one or more mutations are further capable of providing one or more traits selected from the group consisting of:
   A. a reduced level of nitrite,
   B. an increased level of oxygen radicle absorbance capacity (ORAC), and
   C. an increased level of one or more antioxidants;
   D. wherein said reduced or increased level is compared to said cured leaf of a control tobacco plant when grown and cured under comparable.
Embodiment 47. The cured leaf from a modified tobacco plant of any one of embodiments 45 or 46, wherein said one or more mutations comprise a mutation type selected from the group consisting of an insertion, a deletion, an inversion, a duplication, a substitution, and a combination thereof.
Embodiment 48. The cured leaf from a modified tobacco plant of any one of embodiments 45 to 47, wherein said one or more mutations are capable of activating one or more genes encoding a biosynthetic enzyme, a regulatory transcription factor, a transporter, a catabolic enzyme, or a combination thereof, for one or more antioxidants.
Embodiment 49. The cured leaf from a modified tobacco plant of any one of embodiments 45 to 48, wherein said one or more mutations are in one or more genes encoding a biosynthetic enzyme, a regulatory transcription factor, a transporter, a catabolic enzyme, or a combination thereof, for one or more antioxidants selected from the group consisting of anthocyanidin, flavanone, flavanol, flavone, flavonol, isoflavone, hydroxybenzoic acid, hydroxycinnamic acid, ellagitannin, stibene, lignan, carotenoids, and glycyrrhzin.
Embodiment 50. The cured leaf from a modified tobacco plant of any one of embodiments 45 to 49, wherein said one or more mutations are in one or more genes encoding a biosynthetic enzyme, a regulatory transcription factor, a transporter, a catabolic enzyme, or a combination thereof, for one or more antioxidants selected from the group consisting of Delphnidin, Cyanidin, Procyanidin, Prodelphinidin, Hesperetin, Naringenin, Catechin, Epicatechin, Apigenin, Luteonin, Quercetin, Myricetin, Rutin, Genistein, Daidzein, Gallic acid, Vanillic acid, Protocatechuic acid, Ferunic acid, Cinnamic acid, Coumeric acid, Chlorogenic acid, Coffeic acid, ferulic acid, Sanguiin, Resveratrol, Sesamin, Caretonoids, and Vitamin C.
Embodiment 51. The cured leaf from a modified tobacco plant of any one of embodiments 45 to 50, wherein said one or more mutations are introduced via a system selected from the group consisting of chemical mutagenesis, irradiation mutagenesis, transposon mutagenesis, Agrobacterium-mediated transformation, protoplast transformation, electroporation, ballistic transformation, a meganuclease, a zinc-finger nuclease (ZFN), a transcription activator-like effector nuclease (TALEN), a clustered regularly-interspaced short palindromic repeats (CRISPR)/Cas9 system, a CRISPR/Cpf1 system, a CRISPR/Csm1 system, and a combination thereof.
Embodiment 52. The modified tobacco plant of any one of embodiments 45 to 51, one or more mutations are in a gene encoding a polypeptide having at least 50%, 55%, 60%, 65%, 70%, 75%, 80%, 85%, 90%, 95%, 97%, 98%, 99%, or 100% identity to a sequence selected from the group consisting of SEQ ID No. 1 to 23 and 47 to 52.
Embodiment 53. The modified tobacco plant of any one of embodiments 45 to 52, one or more mutations are in a gene comprise a coding sequence having at least 50%, 55%, 60%, 65%, 70%, 75%, 80%, 85%, 90%, 95%, 97%, 98%, 99%, or 100% identity to a sequence selected from the group consisting of SEQ ID No. 24 to 46 and 53 to 58.
Embodiment 54. The modified tobacco plant of any one of embodiments 29 to 44, wherein said modified tobacco plant comprises one or more transgenes capable of providing said reduced level of one or more TSNAs in cured leaf.
Embodiment 55. The cured leaf from a modified tobacco plant of embodiment 54, wherein said one or more transgenes further cause said one or more traits selected from the group consisting of:
   A. a reduced level of nitrite,
   B. an increased level of oxygen radicle absorbance capacity (ORAC), and
   C. an increased level of one or more antioxidants;
   D, wherein said reduced or increased level is compared to said cured leaf from a control tobacco plant when grown and cured under comparable.
Embodiment 56. The modified tobacco plant of any one of embodiments 54 or 55, wherein said one or more transgenes comprises a promoter selected from the group consisting of a constitutive promoter, an inducible promoter, and a tissue-preferred promoter.
Embodiment 57. The modified tobacco plant of any one of embodiments 54 to 56, wherein said one or more transgenes comprises a leaf-specific promoter, a shoot-specific promoter, or a root-specific promoter.
Embodiment 58. The modified tobacco plant of any one of embodiments 54 to 57, wherein said one or more transgenes encode a biosynthetic enzyme, a regulatory transcription factor, a transporter, a catabolic enzyme, or a combination thereof, for one or more antioxidants selected from the group consisting of anthocyanidin, flavanone, flavanol, flavone, flavonol, isoflavone, hydroxybenzoic acid, hydroxycinnamic acid, ellagitannin, stibene, lignan, carotenoids, and glycyrrhzin.
Embodiment 59. The modified tobacco plant of any one of embodiments 54 to 58, wherein said one or more transgenes encode a biosynthetic enzyme, a regulatory transcription factor, a transporter, a catabolic enzyme, or a combination thereof, for one or more antioxidants selected from the group consisting of Delphnidin, Cyanidin, Procyanidin, Prodelphinidin, Hesperetin, Naringenin, Catechin, Epicatechin, Apigenin, Luteonin, Quercetin, Myricetin, Rutin, Genistein, Daidzein, Gallic acid, Vanillic acid, Protocatechuic acid, Ferunic acid, Cinnamic acid, Coumeric acid, Chlorogenic acid, Coffeic acid, ferulic acid, Sanguiin, Resveratrol, Sesamin, Caretonoids, and Vitamin C.
Embodiment 60. The modified tobacco plant of any one of embodiments 54 to 59, wherein said one or more transgenes encodes one or more polypeptides having at least 50%, 55%, 60%, 65%, 70%, 75%, 80%, 85%, 90%, 95%, 97%, 98%, 99%, or 100% identity to a sequence selected from the group consisting of SEQ ID No. 1 to 23 and 47 to 52.
Embodiment 61. The modified tobacco plant of any one of embodiments 54 to 60, wherein said one or more transgenes targets one or more endogenous genes having at least 50%, 55%, 60%, 65%, 70%, 75%, 80%, 85%, 90%, 95%, 97%, 98%, 99%, or 100% identity to a sequence selected from the group consisting of SEQ ID No. 24 to 46 and 53 to 58.
Embodiment 62. The modified tobacco plant of any one of embodiments 54 to 61, wherein said one or more transgenes comprises one or more polynucleotide sequences encoding one or more polypeptides having at least 50%, 55%, 60%, 65%, 70%, 75%, 80%, 85%, 90%, 95%, 97%, 98%, 99%, or 100% identity to a sequence selected from the group consisting of SEQ ID No. 1 to 23 and 47 to 52.
Embodiment 63. The modified tobacco plant of any one of embodiments 54 to 62, wherein said one or more transgenes comprise a sequence having at least 50%, 55%, 60%, 65%, 70%, 75%, 80%, 85%, 90%, 95%, 97%, 98%, 99%, or 100% identity to a sequence selected from the group consisting of SEQ ID No. 24 to 46 and 53 to 58.
Embodiment 64. The modified tobacco plant of any one of embodiments 54 to 63, wherein said one or more transgenes comprise a sequence having at least 50%, 55%, 60%, 65%, 70%, 75%, 80%, 85%, 90%, 95%, 97%, 98%, 99%, or 100% identity to a sequence selected from the group consisting of SEQ ID No. 24 to 46 and 53 to 58.
Embodiment 65. The modified tobacco plant of any one of embodiments 54 to 64, wherein said modified tobacco plant is a cis-genic plant.
Embodiment 66. The modified tobacco plant of any one of embodiments 29 to 65, wherein said modified tobacco plant has a similar or higher leaf yield compared to said control tobacco plant when grown and cured under comparable conditions.
Embodiment 67. The modified tobacco plant of embodiment 66, wherein said higher leaf yield is at least 0.5%, 1%, 2.5%, 5%, 10%, 15%, or at least 20% higher.
Embodiment 68. The modified tobacco plant of any one of embodiments 29 to 66, wherein said modified tobacco plant has a similar plant height compared to said control tobacco plant when grown and cured under comparable conditions.
Embodiment 69. The modified tobacco plant of embodiment 68, wherein said similar plant height is within 1%, 5%, 10%, 20%, or 25%.
Embodiment 70. The modified tobacco plant of any one of embodiments 29 to 68, wherein said modified tobacco plant has a similar cured leaf chemistry profile compared to said control tobacco plant when grown and cured under comparable conditions.
Embodiment 71. The modified tobacco plant of any one of embodiments 29 to 68, wherein said modified tobacco plant produces cured leaf that has a similar or higher USDA grade index value compared to cured leaf from said control tobacco plant when grown and cured under comparable conditions.
Embodiment 72. The modified tobacco plant of embodiment 45 or 54, wherein said modified tobacco plant is homozygous for said one or more transgenes or said one or more mutations.
Embodiment 73. The modified tobacco plant of any one of embodiments 45, 54, or 72, wherein said modified tobacco plant is hemizygous for said one or more transgenes or said one or more mutations.
Embodiment 74. The modified tobacco plant of any one of embodiments 45, 54, 72, or 73, wherein said modified tobacco plant is heterozygous for said one or more transgenes or said one or more mutations.
Embodiment 75. The modified tobacco plant of any one of embodiments 29 to 74, wherein said modified tobacco plant further comprises reduced nicotine demethylase activity compared to said control plant when grown and cured under comparable conditions.
Embodiment 76. The modified tobacco plant of embodiment 75, wherein said tobacco plant comprises at least one mutation in a nicotine demethylase gene selected from the group consisting of CYP82E4, CYP82E5, CYP82E10, and a combination thereof.
Embodiment 77. The cured leaf from a modified tobacco plant of any one of embodiments 29 to 75, wherein said modified tobacco plant further comprises a reduced level of total alkaloids compared to said cured leaf from a control plant when grown and cured under comparable conditions.
Embodiment 78. The cured leaf from a modified tobacco plant of any one of embodiments 29 to 77, wherein said modified tobacco plant further comprises a substantially similar level of total alkaloids compared to said cured leaf from a control plant when grown and cured under comparable conditions.
Embodiment 79. The cured leaf from a modified tobacco plant of any one of embodiments 29 to 78, wherein said modified tobacco plant further comprises a reduced level of nicotine compared to said cured leaf from a control plant when grown and cured under comparable conditions.
Embodiment 80. The cured leaf from a modified tobacco plant of any one of embodiments 29 to 79, wherein said modified tobacco plant further comprises a substantially similar level of nicotine compared to said cured leaf from a control plant when grown and cured under comparable conditions.
Embodiment 81. The modified tobacco plant of embodiment any one of embodiments 29 to 80, wherein said modified plant is selected from the group consisting of a flue-cured variety, a Burley variety, a Maryland variety, a dark variety, and an Oriental variety.
Embodiment 82. The modified tobacco plant of any one of embodiments 29 to 81, wherein said modified tobacco plant is selected from the group consisting a BU 64 plant, a CC 101 plant, a CC 200 plant, a CC 13 plant, a CC 27 plant, a CC 33 plant, a CC 35 plant, a CC 37 plant, a CC 65 plant, a CC 67 plant, a CC 301 plant, a CC 400 plant, a CC 500 plant, CC 600 plant, a CC 700 plant, a CC 800 plant, a CC 900 plant, a CC 1063 plant, a Coker 176 plant, a Coker 319 plant, a Coker 371 Gold plant, a Coker 48 plant, a CU 263 plant, a DF911 plant, a Galpao plant, a GL 26H plant, a GL 338 plant, a GL 350 plant, a GL 395 plant, a GL 600 plant, a GL 737 plant, a GL 939 plant, a GL 973 plant, a GF 157 plant, a GF 318 plant, an RJR 901 plant, an HB 04P plant, a K 149 plant, a K 326 plant, a K 346 plant, a K 358 plant, a K394 plant, a K 399 plant, a K 730 plant, an NC 196 plant, an NC 37NF plant, an NC 471 plant, an NC 55 plant, an NC 92 plant, an NC2326 plant, an NC 95 plant, an NC 925 plant, a PVH 1118 plant, a PVH 1452 plant, a PVH 2110 plant, a PVH 2254 plant, a PVH 2275 plant, a VA 116 plant, a VA 119 plant, a KDH 959 plant, a KT 200 plant, a KT204LC plant, a KY 10 plant, a KY 14 plant, a KY 160 plant, a KY 17 plant, a KY 171 plant, a KY 907 plant, a KY 907LC plant, a KTY14×L8 LC plant, a Little Crittenden plant, a McNair 373 plant, a McNair 944 plant, a male sterile KY 14×L8 plant, a Narrow Leaf Madole plant, a MS KY171 plant, a Narrow Leaf Madole (phph) plant, a MS Narrow Leaf Madole plant, a MS TND950 plant, a PD 7302LC plant, a PD 7305LC plant, a PD 7309LC plant, a PD 7312LC plant, a PD 7318LC plant, a PD 7319LC plant, a MSTKS 2002 plant, a TKF 2002 plant, a TKF 6400 plant, a TKF 4028 plant, a TKF 4024 plant, a KT206LC plant, a KT209LC plant, a KT210LC plant, a KT212LC plant, an NC 100 plant, an NC 102 plant, an NC 2000 plant, an NC 291 plant, an NC 297 plant, an NC 299 plant, an NC 3 plant, an NC 4 plant, an NC 5 plant, an NC 6 plant, an NC7 plant, an NC 606 plant, an NC 71 plant, an NC 72 plant, an NC 810 plant, an NC BH 129 plant, an NC 2002 plant, a Neal Smith Madole plant, an OXFORD 207 plant, a ‘Perique’ plant, a PVH03 plant, a PVH09 plant, a PVH19 plant, a PVH50 plant, a PVH51 plant, an R 610 plant, an R 630 plant, an R 7-11 plant, an R 7-12 plant, an RG 17 plant, an RG 81 plant, an RG H51 plant, an RGH 4 plant, an RGH 51 plant, an RS 1410 plant, a Speight 168 plant, a Speight 172 plant, a Speight 179 plant, a Speight 210 plant, a Speight 220 plant, a Speight 225 plant, a Speight 227 plant, a Speight 234 plant, a Speight G-28 plant, a Speight G-70 plant, a Speight H-6 plant, a Speight H20 plant, a Speight NF3 plant, a TI 1406 plant, a TI 1269 plant, a TN 86 plant, a TN86LC plant, a TN 90 plant, a TN90LC plant, a TN 97 plant, a TN97LC plant, a TN D94 plant, a TN D950 plant, a TR (Tom Rosson) Madole plant, a VA 309 plant, and a VA 359 plant.
Embodiment 83. The modified tobacco plant of any one of embodiments 29 to 82, wherein said modified tobacco plant is a hybrid.
Embodiment 84. The modified tobacco plant of any one of embodiments 29 to 83, wherein said modified tobacco plant is male sterile or cytoplasmically male sterile (CMS).
Embodiment 85. The modified tobacco plant of any one of embodiments 29 to 84, wherein said modified tobacco plant is female sterile.
Embodiment 86. The cured tobacco leaf or modified tobacco plant of any one of embodiments 29 to 85, wherein said reduced or increased level is within about 10%, within about 20%, within about 30%, within about 40%, within about 50%, within about 60%, within about 70%, within about 80%, within about 90%, within about 92%, within about 94%, within about 95%, within about 96%, within about 97%, within about 98%, or within about 99% lower or higher than the level in said control cured tobacco leaf or tobacco plant when grown and cured under comparable conditions.
Embodiment 87. The cured tobacco leaf or modified tobacco plant of any one of embodiments 29 to 86, wherein said reduced or increased level is within about 1 fold, within about 2 folds, within about 3 folds, within about 4 folds, within about 5 folds, within about 6 folds, within about 7 folds, within about 8 folds, within about 9 folds, within about 10 folds, within about 15 folds, within about 20 folds, within about 25 folds, or within about 30 folds lower or higher than the level in said control cured tobacco leaf or tobacco plant when grown and cured under comparable conditions.
Embodiment 88. The cured tobacco leaf or modified tobacco plant of any one of embodiments 29 to 87, wherein said reduced or increased level is at least about 10%, at least about 20%, at least about 30%, at least about 40%, at least about 50%, at least about 60%, at least about 70%, at least about 80%, at least about 90%, at least about 92%, at least about 94%, at least about 95%, at least about 96%, at least about 97%, at least about 98%, or at least about 99% lower or higher than the level in said control cured tobacco leaf or tobacco plant when grown and cured under comparable conditions.
Embodiment 89. The cured tobacco leaf or modified tobacco plant of any one of embodiments 29 to 88, wherein said reduced or increased level is at least about 1 fold, at least about 2 folds, at least about 3 folds, at least about 4 folds, at least about 5 folds, at least about 6 folds, at least about 7 folds, at least about 8 folds, at least about 9 folds, at least about 10 folds, at least about 15 folds, at least about 20 folds, at least about 25 folds, or at least about 30 folds lower or higher than the level in said control cured tobacco leaf or tobacco plant when grown and cured under comparable conditions.
Embodiment 90. The cured tobacco leaf or modified tobacco plant of any one of embodiments 29 to 89, wherein said reduced or increased level is about 10%, about 20%, about 30%, about 40%, about 50%, about 60%, about 70%, about 80%, about 90%, about 92%, about 94%, about 95%, about 96%, about 97%, about 98%, or about 99% lower or higher than the level in said control cured tobacco leaf or tobacco plant when grown and cured under comparable conditions.
Embodiment 91. The cured tobacco leaf or modified tobacco plant of any one of embodiments 29 to 90, wherein said reduced or increased level is about 1 fold, about 2 folds, about 3 folds, about 4 folds, about 5 folds, about 6 folds, about 7 folds, about 8 folds, about 9 folds, about 10 folds, about 15 folds, about 20 folds, about 25 folds, or about 30 folds lower or higher than the level in said control cured tobacco leaf or tobacco plant when grown and cured under comparable conditions.
Embodiment 92. The cured tobacco leaf or modified tobacco plant of any one of embodiments 29 to 91, wherein said reduced or increased level is about 1-2 folds, about 2-3 folds, about 3-4 folds, about 4-5 folds, about 5-6 folds, about 6-7 folds, about 7-8 folds, about 8-9 folds, about 9-10 folds, about 10-15 folds, about 15-20 folds, about 20-25 folds, about 25-30 folds, or about 30-50 folds lower or higher than the level in said control cured tobacco leaf or tobacco plant when grown and cured under comparable conditions.
Embodiment 93. The cured tobacco leaf or modified tobacco plant of any one of embodiments 29 to 92, wherein said reduced or increased level is about 1-10 folds, about 2-10 folds, about 3-10 folds, about 4-10 folds, about 5-10 folds, about 6-10 folds, about 7-10 folds, about 8-10 folds, about 9-10 folds, about 10-50 folds, about 15-50 folds, about 20-50 folds, about 25-50 folds, or about 30-50 folds lower or higher than the level in said cured tobacco leaf or control tobacco plant when grown and cured under comparable conditions.
Embodiment 94. A tobacco leaf of the modified tobacco plant of any one of embodiments 29 to 93.
Embodiment 95. The tobacco leaf of embodiment 94, wherein said tobacco leaf is cured tobacco leaf.
Embodiment 96. The tobacco leaf of any one of embodiments 94 or 95, wherein said cured tobacco leaf is air-cured, fire-cured, sun-cured, or flue-cured.
Embodiment 97. The cured tobacco leaf of any one of embodiments 94 to 96 comprising less than 2 ppm total TSNAs, wherein the level of said total TSNAs is measured based on a freeze-dried cured leaf sample using liquid chromatograph with tandem mass spectrometry (LC/MS/MS).
Embodiment 98. The cured tobacco leaf of any one of embodiments 94 to 97, wherein said cured tobacco leaf comprises less than 2, less than 1.8, less than 1.5, less than 1.2, less than 1.0, less than 0.8, less than 0.6, less than 0.4, less than 0.3, less than 0.2, less than 0.15, less than 0.1, or less than 0.05 ppm total TSNAs.
Embodiment 99. The cured tobacco leaf of any one of embodiments 94 to 98, wherein said cured tobacco leaf comprises between 2 and 0.05, between 1.8 and 0.05, between 1.5 and 0.05, between 1.2 and 0.05, between 1.0 and 0.05, between 0.8 and 0.05, between 0.6 and 0.05, between 0.4 and 0.05, between 0.3 and 0.05, between 0.2 and 0.05, between 0.15 and 0.05, or between 0.1 and 0.05 ppm total TSNAs.
Embodiment 100. The cured tobacco leaf of any one of embodiments 94 to 99, wherein said cured tobacco leaf comprises between 2 and 0.05, between 1.8 and 0.1, between 1.5 and 0.15, between 1.2 and 0.2, between 1.0 and 0.3, between 0.8 and 0.4, or between 0.6 and 0.5 ppm total TSNAs.
Embodiment 101. The cured tobacco leaf of any one of embodiments 94 to 100, comprising less than 0.08 ppm 4-methylnitrosoamino-1-(3-pyridyl)-1-butanone (NNK), wherein the level of said total TSNAs is measured based on a freeze-dried cured leaf sample using liquid chromatograph with tandem mass spectrometry (LC/MS/MS).
Embodiment 102. A tobacco product comprising cured leaf from the modified tobacco plant of any one of embodiments 29 to 85.
Embodiment 103. The tobacco product of embodiment 102, wherein said tobacco product is selected from the group consisting of a cigarette, a kretek, a bidi cigarette, a cigar, a cigarillo, a non-ventilated cigarette, a vented recess filter cigarette, pipe tobacco, snuff, snus, chewing tobacco, moist smokeless tobacco, fine cut chewing tobacco, long cut chewing tobacco, pouched chewing tobacco product, gum, a tablet, a lozenge, an e-liquid for a vaping device, and a dissolving strip.
Embodiment 104. A seed giving rise to the modified tobacco plant of any one of embodiments 29 to 85.
Embodiment 105. A method comprising:
   A. planting the seed of embodiment 104; and
   B. growing a tobacco plant from said seed.
Embodiment 106. A method comprising preparing a tobacco product using cured tobacco leaf from the modified tobacco plant of any one of embodiments 29 to 85.
Embodiment 107. A method of reducing the level of one or more TSNAs in cured leaf from a tobacco plant, said method comprising increasing the level of one or more antioxidants in said tobacco plant by expressing a biosynthetic enzyme, a regulatory transcription factor, a transporter, a catabolic enzyme, or a combination thereof, for said one or more antioxidants.
Embodiment 108. The method of embodiment 107, wherein said one or more antioxidants are selected from the group consisting of anthocyanidin, flavanone, flavanol, flavone, flavonol, isoflavone, hydroxybenzoic acid, hydroxycinnamic acid, ellagitannin, stibene, lignan, carotenoids, and glycyrrhzin.
Embodiment 109. The method of any one of embodiment s 107 or 108, wherein said one or more antioxidants are selected from the group consisting of Delphnidin, Cyanidin, Procyanidin, Prodelphinidin, Hesperetin, Naringenin, Catechin, Epicatechin, Apigenin, Luteonin, Quercetin, Myricetin, Rutin, Genistein, Daidzein, Gallic acid, Vanillic acid, Protocatechuic acid, Ferunic acid, Cinnamic acid, Coumeric acid, Chlorogenic acid, Coffeic acid, ferulic acid, Sanguiin, Resveratrol, Sesamin, Caretonoids, and Vitamin C.
Embodiment 110. The method of any one of embodiment s 107 to 109, wherein said method does not substantially reduce the level of total alkaloids in said cured leaf from a tobacco plant.
Embodiment 111. The method of one of embodiment s 107 to 110, wherein said method does not substantially reduce the level of nicotine in said cured leaf from a tobacco plant.
Embodiment 112. A method for producing a tobacco plant comprising:
   A. crossing at least one tobacco plant of a first tobacco variety with at least one tobacco plant of a second tobacco variety, wherein said at least one tobacco plant of said first tobacco variety is the modified tobacco plant of any one of embodiments 29 to 93; and
   B. selecting for cured leaf from a progeny tobacco plant comprising a reduced level of one or more tobacco-specific nitrosamines (TSNAs) and further comprising one or more traits selected from the group consisting of
      i. a reduced level of nitrite,
      ii. an increased level of oxygen radical absorbance capacity (ORAC), and
      iii. an increased level of one or more antioxidants;
      iv, wherein said reduced or increased level is compared to cured leaf from a control tobacco plant of the same cross grown and cured under comparable conditions.

### EXAMPLES

### Example 1. Plant Transformation

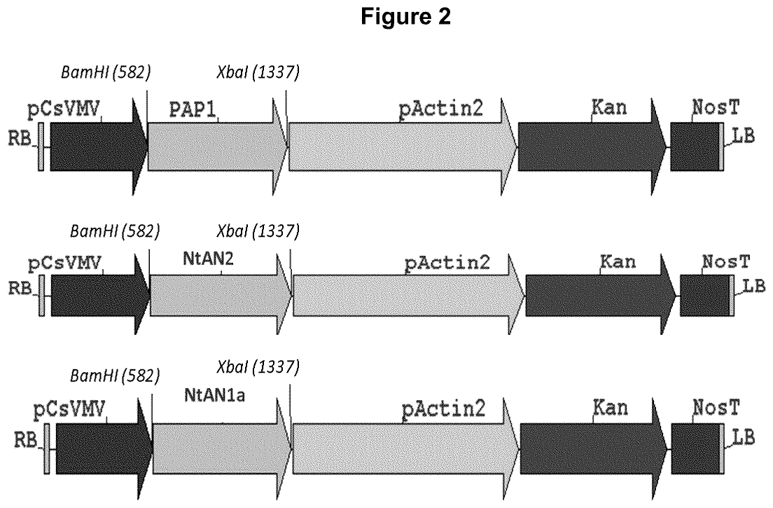

Tobacco plants overexpressing a gene of interest are generated via Agrobacterium-mediated transformation. An expression vector, p45-2-7 (FIG. 2), is used as a backbone to generate multiple transformation vectors. p45-2-7 contains a CsVMV promoter, a NOS terminator, and a cassette comprising a kanamycin selection marker (NPT II) operably linked to an Actin2 promoter and a NOS terminator. Nucleic acid vectors comprising transgenes of interest are introduced into tobacco leaf discs via Agrobacterium transformation. See, for example, Mayo et al., 2006, Nat Protoc. 1:1105-11 and Horsch et al., 1985, Science 227:1229-1231.

Narrow Leaf Madole (NLM) tobacco plants are grown in Magenta™ GA-7 boxes and leaf discs are cut and placed into Petri plates. Agrobacterium tumefaciens cells comprising a transformation vector are collected by centrifuging a 20 mL cell suspension in a 50 mL centrifuge tube at 3500 RPM for 10 minutes. The supernatant is removed and the Agrobacterium tumefaciens cell pellet is re-suspended in 40 mL liquid re-suspension medium. Tobacco leaf, avoiding the midrib, are cut into eight 0.6 cm discs with a #15 razor blade and placed upside down in a Petri plate. A thin layer of Murashige & Skoog with B5 vitamins liquid re-suspension medium is added to the Petri plate and the leaf discs are poked uniformly with a fine point needle. About 25 mL of the Agrobacterium tumefaciens suspension is added to the Petri plate and the leaf discs are incubated in the suspension for 10 minutes.

Leaf discs are transferred to co-cultivation Petri plates (½ MS medium) and discs are placed upside down in contact with filter paper overlaid on the co-cultivation TOM medium (MS medium with 20 g/L sucrose; 1 mg/L indole-3-acetic acid; and 2.5 mg/L 6-benzyl aminopurine (BAP)). The Petri plate is sealed with parafilm prior to incubation in dim light (60-80 mE/ms) with 18 hours on, 6 hours off photoperiods at 24 degrees Celsius for three days. After incubation, leaf discs are transferred to regeneration/selection TOM K medium Petri plates (TOM medium plus 300 mg/L kanamycin). Leaf discs are sub-cultured bi-weekly to fresh TOM K medium in dim light with 18 hours on, 6 hours off photoperiods at 24 degrees Celsius until shoots become excisable. Shoots from leaf are removed with forceps and inserted in MS basal medium with 100 mg/L kanamycin. Shoots on MS basal medium with 100 mg/L kanamycin are incubated at 24 degrees Celsius with 18 hours on, 6 hours off photoperiods with high intensity lighting (6080 mE/ms) to induce rooting.

When plantlets containing both shoots and roots grow large enough (e.g., reach approximately half the height of a Magenta™ GA-7 box), they are transferred to soil. Established seedlings are transferred to a greenhouse for further analysis and to set seed. Control plants are either NLM plants that have not been transformed or NLM plants that have been transformed with an empty p45-2-7 vector.

### Example 2. AtPAP1 Overexpressing Plants Comprise Reduced TSNAs

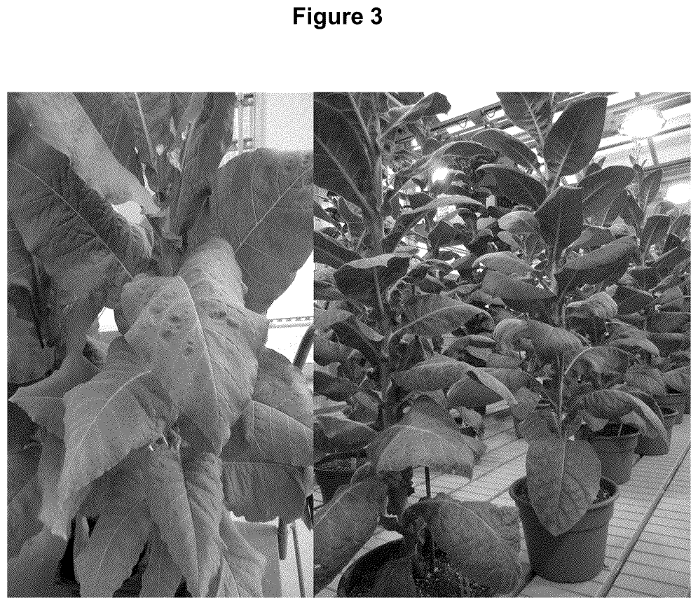

Tobacco plants overexpressing AtPAP1 are generated via Agrobacterium-mediated transformation. AtPAP1, comprising SEQ ID NO:46, is incorporated into an overexpression vector and transformed into tobacco as described in Example 1. After transformation, established seedlings are transferred to a greenhouse for further analysis and to set seed. Transformed plants are developmentally similar to control plants except that they exhibit a purple color due to anthocyanin accumulation as shown in FIG. 3.

The effect of AtPAP1 overexpression on TSNA levels is determined for five T1 AtPAP1 overexpression lines (six plants each) after cultivation in the greenhouse. At flowering, the plants are topped. After four to six weeks, plants are harvested and leaf is cured in PGC chambers. Cured leaf samples are freeze dried and crushed to 1 mm.

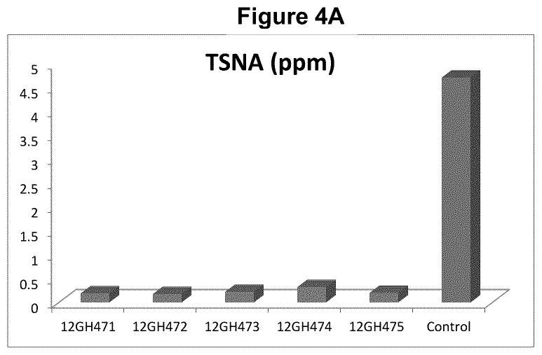

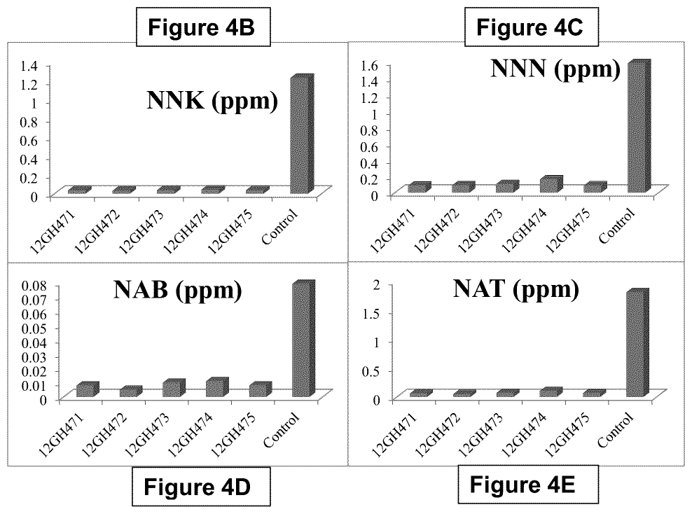

The amounts of four TSNAs are measured: N′-nitrosonornicotine (NNN), 4-methylnitrosoamino-1-(3-pyridyl)-1-butanone (NNK), N′-nitrosoanatabine (NAT) and N′-nitrosoanabasine (NAB) are measured. For TSNA analysis, 750 mg of crushed, freeze-dried leaf is added to 30 mls of 10 mM ammonium acetate. After incubation in a shaker for 30 minutes, approximately 4 mls of sample is transferred into disposable culture tubes containing 0.25 ml of concentrated ammonium hydroxide. The sample is vortexed briefly and 1.5 mls is added to a prewashed and conditioned extraction cartridge with a flow rate of 1 to 2 drops per second. Analytes are eluted from the sample using 1.5 mls of 70:30 methanol with 0.1% acetic acid. Samples are analyzed using liquid chromatography with tandem mass spectrometry (LC/MS/MS). Measurements of NNN, NNK, NAB, and NAT in AtPAP1 overexpressing plants is shown in Table 1. Total TSNA levels are considerably reduced in AtPAP1 plants as shown in FIG. 4A. Considerable reductions in NNK levels (FIG. 4B), NNN levels (FIG. 4C), NAB levels (FIG. 4D), and NAT levels (FIG. 4E) are also observed.

TABLE 1
TSNA levels in AtPAP1 overexpression plants are reduced compared to controls.

| | | | | | | Total | |
|---|---|---|---|---|---|---|---|
| | | NNN | NNK | NAB | NAT | TSNA | % TSNA |
| Plant ID | Variety | (ppm) | (ppm) | (ppm) | (ppm) | (ppm) | Reduction |
| 12GH471 | t-NL Madole | 0.087 | 0.035 | 0.008 | 0.059 | 0.189 | 95.98 |
| | LC T821 | | | | | | |
| | (PAP1 OEX) | | | | | | |
| 12GH472 | t-NL Madole | 0.091 | 0.031 | 0.005 | 0.048 | 0.175 | 96.28 |
| | LC T824 | | | | | | |
| | (PAP1 OEX) | | | | | | |
| 12GH473 | t-NL Madole | 0.105 | 0.037 | 0.01 | 0.069 | 0.221 | 95.30 |
| | LC T827 | | | | | | |
| | (PAP1 OEX) | | | | | | |
| 12GH474 | t-NL Madole | 0.164 | 0.043 | 0.011 | 0.103 | 0.321 | 93.18 |
| | LC T827 | | | | | | |
| | (PAP1 OEX) | | | | | | |
| 12GH475 | t-NL Madole | 0.089 | 0.036 | 0.008 | 0.067 | 0.2 | 95.75 |
| | LC T836 | | | | | | |
| | (PAP1 OEX) | | | | | | |
| Control | NL Madole | 1.581 | 1.232 | 0.079 | 1.812 | 4.704 | — |

### Example 3. AtPAP1 Overexpressing Plants Exhibit Increased Oxygen Radical Absorbance Capacity

The effect of AtPAP1 overexpression on oxidative capacity is determined for five T1 AtPAP1 overexpression lines (six plants each) after cultivation in the greenhouse. Oxygen Radical Absorbance Capacity (ORAC) is measured to determine antioxidant activity in AtPAP1 overexpressing plants. Quenching of a Progallol Red (PGR) florescent probe is used to determine the ORAC measurement according to manufactures instruction (BioTek, Winooski, VT). Antioxidants are extracted from crushed tissue samples with a methanol/HCL extraction buffer (6/1, v/v). The samples are incubated for 30 minutes at 37° C. in a reaction mixture containing 75 mM phosphate buffer, pH 7.4, and 5 μM PGR. After incubation, 37° C. AAPH solution is added to the reaction mixture to a final concentration of 10 mM. Controls with all the solution components, but without the tissue samples, are used for comparison.

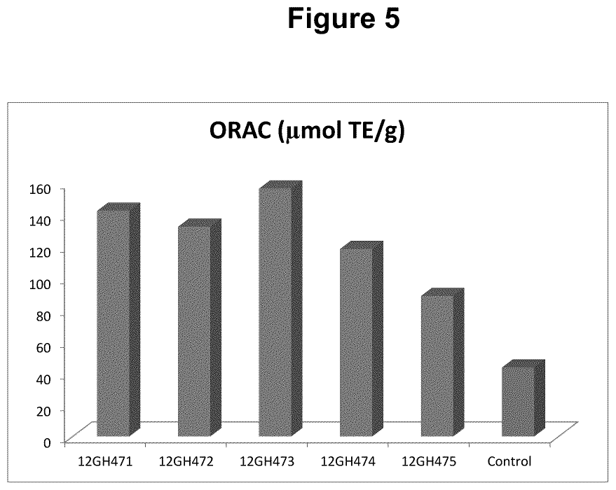

Reaction and control samples are shaken and the absorption (A) is recorded every 30 seconds for 180 minutes. The kinetic values are recorded as A/Aₜᵢₘₑ₀. ORAC scores are determined based on the Area Under Curve (AUC) values determined scores from the sample and blank. ORAC scores are assessed for all time-points until the A/Aₜᵢₘₑ₀ reaches a value of 0.2 using MicroCal Origin (R17.0, Boston, MA). ORAC values are recorded in FIG. 5 demonstrating increased ORAC values in AtPAP1 overexpression lines.

### Example 4. Alkaloid Levels in Tobacco Plants Expressing AtPAP1

The effect of AtPAP1 overexpression on total alkaloid levels is determined for five T1 AtPAP1 overexpression lines (six plants each) after cultivation in the greenhouse. The levels of the alkaloids nicotine, nornicotine, anatabine, and anabasine are determined with Gas Chromatography followed by Mass Spectrometry (GC-MS). For example, measurement of anatabine is performed by mixing one gram of cured leaf tissue with 10 mls of 2N NaOH, followed by incubation at room temperature for fifteen minutes. Anatabine is then extracted by addition of 50 mls of 0.04% quinolone (w/v) dissolved in methyl-tert-butyl ether followed by rotation on a linear shaker for three hours. After phase separation, alkaloid levels are determined using an Agilent 6890 Gas Chromatograph and an Agilent 5973N Mass Spectrometer. The results of measurements for the alkaloids nicotine, nornicotine, anatabine, and anabasine are recorded in Table 2.

TABLE 2
Alkaloid levels in AtPAP1 overexpressing plants
are mildly reduced compared to controls.

| | | Nicotine | Nornicotine | Anabasine | Anatabine |
|---|---|---|---|---|---|
| Plant ID | Variety | (% by wt) | (% by wt) | (% by wt) | (% by wt) |
| 12GH471 | t-NL Madole LC | 3.679 | 0.046 | 0.009 | 0.038 |
| | T821 (PAP1 OEX) | | | | |
| 12GH472 | t-NL Madole LC | 3.009 | 0.089 | 0.008 | 0.034 |
| | T824 (PAP1 OEX) | | | | |
| 12GH473 | t-NL Madole LC | 4.323 | 0.073 | 0.011 | 0.048 |
| | T827 (PAP1 OEX) | | | | |
| 12GH474 | t-NL Madole LC | 4.609 | 0.052 | 0.009 | 0.037 |
| | T827 (PAP1 OEX) | | | | |
| 12GH475 | t-NL Madole LC | 3.329 | 0.036 | 0.008 | 0.034 |
| | T836 (PAP1 OEX) | | | | |
| Control | NL Madole | 5.921 | 0.09 | 0.014 | 0.074 |

### Example 5. AtPAP1 Overexpressing Plants Comprise Reduced Nitrite

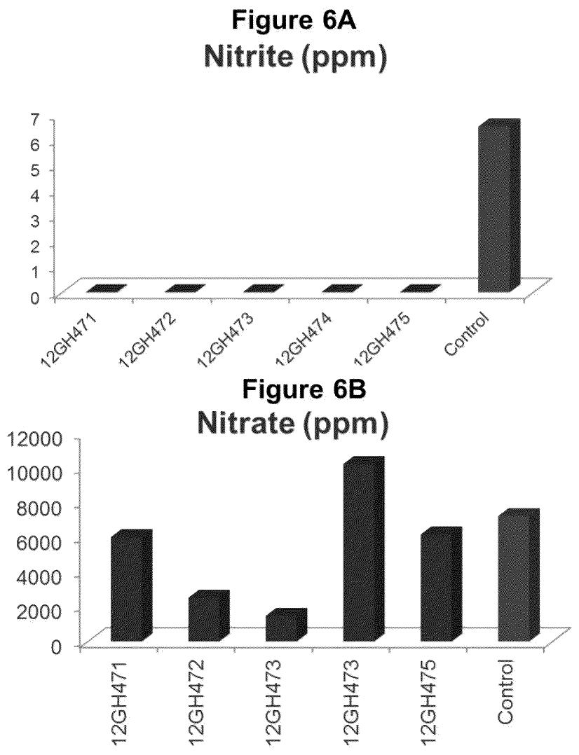

The effect of AtPAP1 overexpression on nitrite and nitrate levels is determined for five T1 AtPAP1 overexpression lines (six plants each) after cultivation in the greenhouse. Cured leaf Samples are prepared as in Example 2 for LC/MS/MS and tested. Nitrite and nitrate levels as shown in FIG. 6A and FIG. 6B. The overexpression of AtPAP1 reduces the level of nitrite but not nitrate.

### Example 6. Reduced Chlorogenic Acid in Tobacco Leaf Correlates with Elevated TSNA Levels

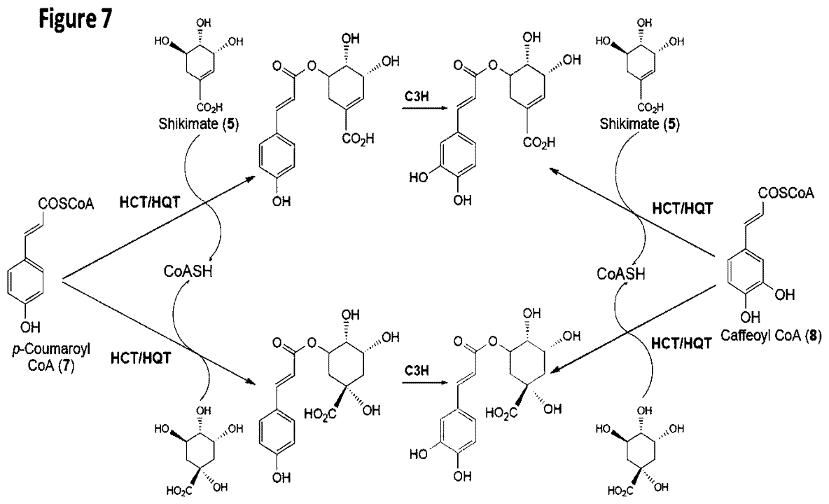

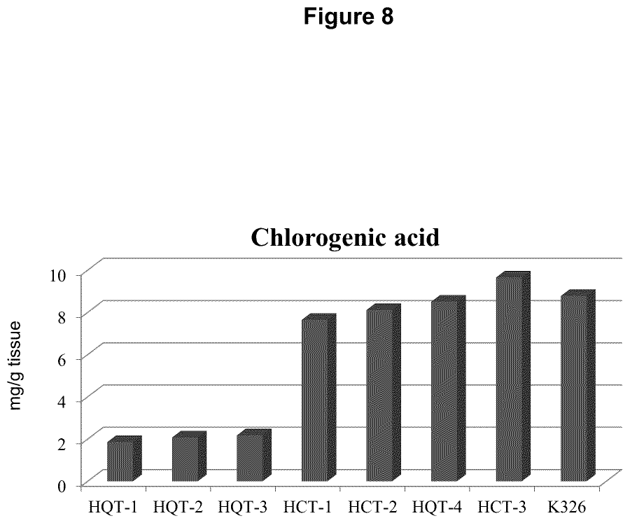

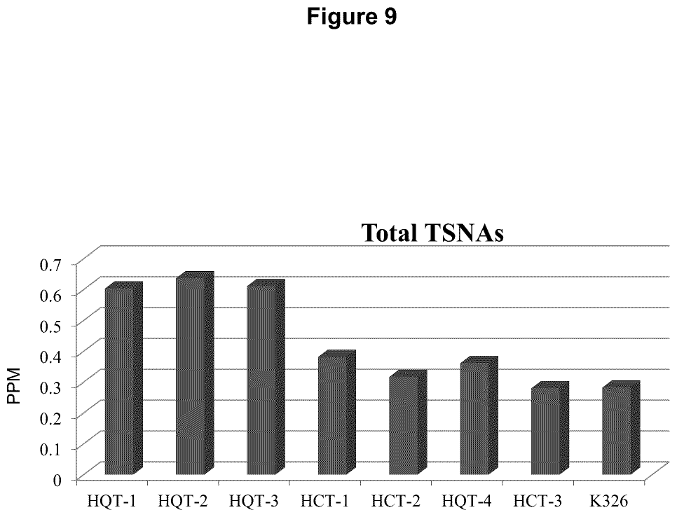

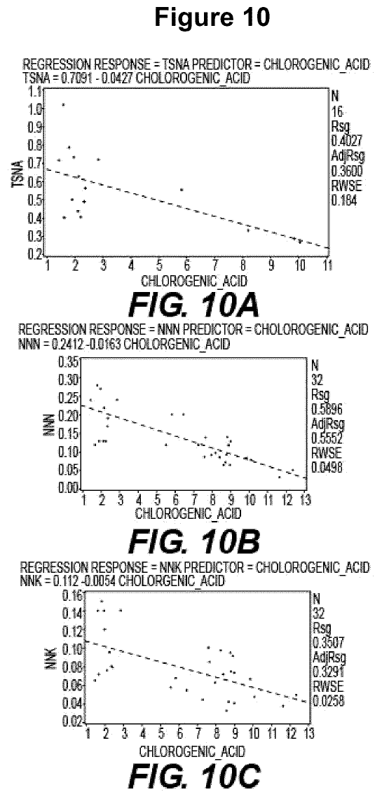

The level of additional antioxidants are modulated to further demonstrate a negative correlation between antioxidants and TSNAs. Reduction of Chlorogenic acid (CGA) levels results in an increase in total TSNAs and total alkaloids. CGA or Caffeoyl quinate is generated from Caffeoyl CoA or p-Coumaroyl CoA through the activity of hydroxycinnamoyl-CoA quinate hydroxycinnamoyl transferase (HQT) and hydroxycinnamoyl-CoA shikimate/quinate hydroxycinnamoyl transferase (HCT) (Payyavula et al., 2015, Plant Biotechnology Journal, Hoffmann et al., 2004, The Plant Cell, FIG. 7). The activity of these enzymes is reduced in tobacco by silencing HCT and HQT with RNAi. Silencing HQT and HCT results in a reduction of CGA as shown in FIG. 8 and an increase in total TSNAs as shown in FIG. 9.

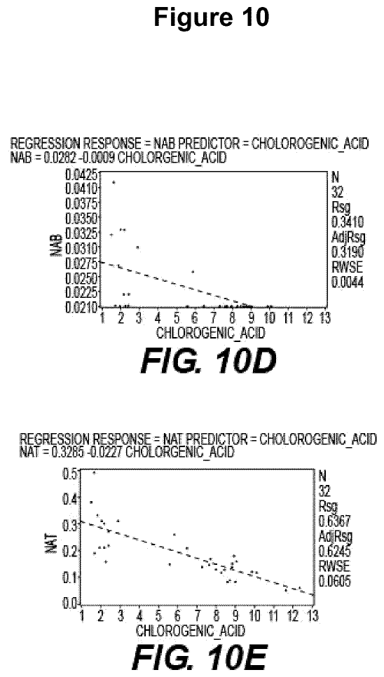

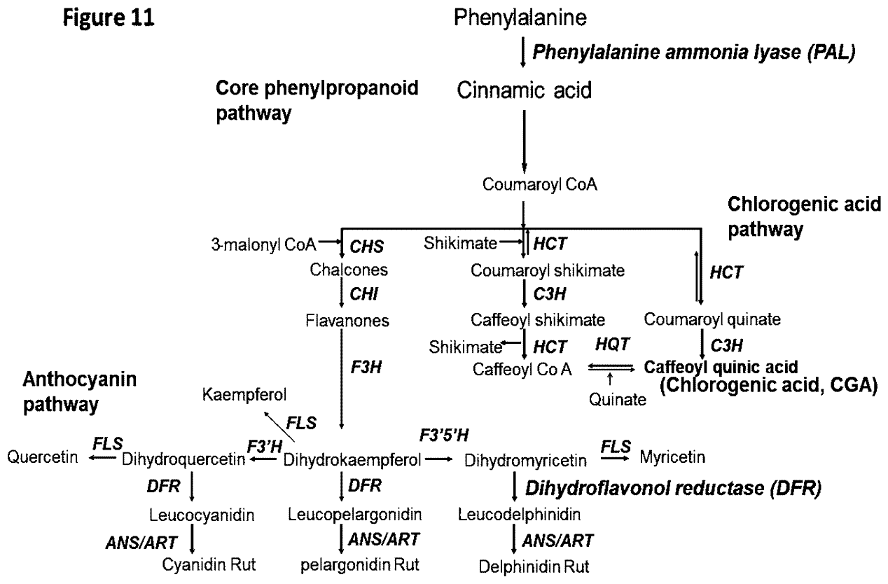

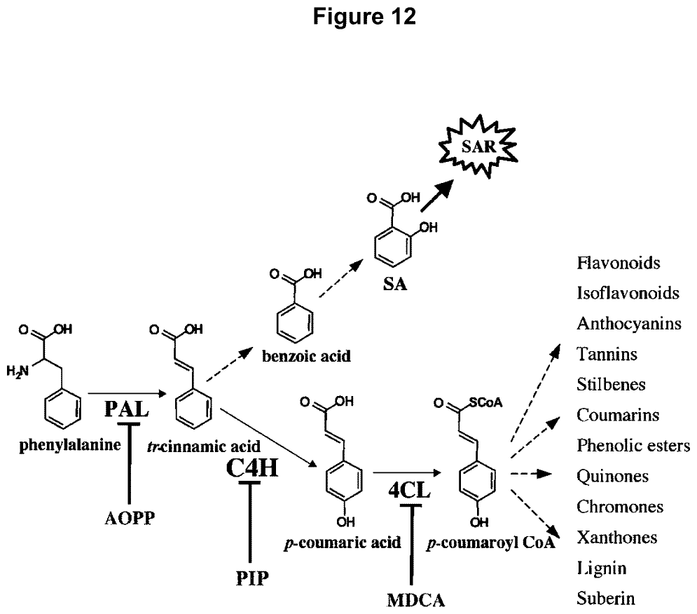

Transformation vectors comprising RNAi constructs are designed to inhibit the expression of tobacco genes that promote the conversion of Caffeoyl CoA or p-Coumaroyl CoA to CGA. Modified tobacco plants and control tobacco plants are created and grown as described in Example 1. Cured leaf samples from the modified tobacco plants are prepared for evaluation of TSNAs, alkaloids, and nitrite/nitrate as described in Examples 2, 4 and 5. Alkaloid levels show mild modulations as shown in Table 3. A negative correlation is observed between CGA levels and TSNA levels, as well as the levels of individual TSNAs (NNN, NNK, NAB, and NNA) as shown in FIG. 10A-E and Table 4. Nitrite levels are unchanged and nitrate levels show reductions compared to controls (Table 4).

TABLE 3
Alkaloid and CGA levels in HCT and HQT RNAi lines.

| | | Nicotine | Nornicotine | Myosmine | Anabasine | Anatabine | |
|---|---|---|---|---|---|---|---|
| | | (% by wt) | (% by wt) | (% by wt) | (% by wt) | (% by wt) | CGA(mg/g) |
| K326 | HQT-1 | 3.90 | 0.10775 | 0.007918 | 0.0217 | 0.109775 | 1.8525 |
| K326 | HQT-2 | 4.10 | 0.112275 | 0.006815 | 0.022825 | 0.106125 | 2.07 |
| K326 | HQT-3 | 4.04 | 0.115 | 0.006995 | 0.021025 | 0.103175 | 2.17 |
| K326 | HCT-1 | 3.59 | 0.095625 | 0.006978 | 0.018425 | 0.091025 | 7.6375 |
| K326 | HCT-2 | 3.45 | 0.083525 | 0.005678 | 0.0178 | 0.085525 | 8.1075 |
| K326 | HQT-4 | 3.93 | 0.0972 | 0.00643 | 0.01985 | 0.10085 | 8.5025 |
| K326 | HCT-3 | 3.28 | 0.07795 | 0.006198 | 0.0154 | 0.074925 | 9.65 |
| K326 | Control | 3.45 | 0.09165 | 0.007213 | 0.018575 | 0.094975 | 8.775 |

TABLE 4
TSNA, CGA, Nitrite and Nitrate levels in HQT and HCT RNAi plants.

| | | LL | LL | LL | LL | LL | LL | LL | |
|---|---|---|---|---|---|---|---|---|---|
| | | NNN | NNK | NAB | NAT | TSNA | Nitrite | Nitrate | |
| | | (ppm) | (ppm) | (ppm) | (ppm) | (ppm) | (ppm) | (ppm) | CGA(mg/g) |
| K326 | HQT-1 | 0.205 | 0.08425 | 0.02675 | 0.2875 | 0.6035 | 0.245 | 3192.5 | 1.8525 |
| K326 | HQT-2 | 0.2175 | 0.09825 | 0.0285 | 0.2925 | 0.63675 | 0.2 | 3257.5 | 2.07 |
| K326 | HQT-3 | 0.195 | 0.1265 | 0.02475 | 0.265 | 0.61125 | 0.22 | 2685 | 2.17 |
| K326 | HCT-1 | 0.13325 | 0.0635 | 0.02 | 0.165 | 0.38175 | 0.2 | 3285 | 7.6375 |
| K326 | HCT-2 | 0.0955 | 0.07025 | 0.02 | 0.13075 | 0.3165 | 0.2225 | 3202.5 | 8.1075 |
| K326 | HQT-4 | 0.11475 | 0.07 | 0.0215 | 0.155 | 0.36125 | 0.215 | 2655 | 8.5025 |
| K326 | HCT-3 | 0.088 | 0.0635 | 0.02 | 0.1085 | 0.28 | 0.2525 | 2945.5 | 9.65 |
| K326 | Control | 0.0885 | 0.0575 | 0.02 | 0.1165 | 0.2825 | 0.2 | 1862.5 | 8.775 |

### Example 7: Increased Antioxidant Capacity in Field Grown AtPAP1 Overexpressing Plants

A Ferric Reducing Antioxidant Power (FRAP) analysis is conducted on field grown tobacco plants overexpressing AtPAP1. AtPAP1 overexpression constructs are transformed into TN90 and Narrow Leaf Madole (NLM) tobacco plants as described in Example 1. Modified and unmodified control plants are grown in a field under standard field conditions. Plants are topped at flowering and leaves for analysis are collected harvest stage (4 weeks) later. At least five plants from two independent transgenic events in both the TN90 background and the NLM background and at least five plants from unmodified TN90 and NLM plants are sampled and tested. 10 mg of freeze dried leaf is taken into an Eppendorf tube and 1500 μl of 80% ethanol is added and sonicated for 10 minutes. After centrifuge, 5-10 μl of supernatant is used to measure antioxidant capacity.

The Ferric Reducing Antioxidant Power (FRAP) method is based on the reduction of complexes of 2,4,6-tripyridyl-s-triazine (TPTZ) with ferric chloride hexahydrate (FeCl3·6H2O) which forms blue ferrous complexes after its reduction (Benzie & Strain, 1996, Analytical Biochemistry, 239, 70-76). Three solutions are used for the assay: Solution 1) 10 mmol·L-1 solution of TPTZ (0.07802 g/25 mL), in 40 mM of hydrochloric acid; Solution 2) 20 mM solution of ferric chloride hexahydrate (0.13513 g/25 mL) in ACS water; Solution 3) 20 mM acetate buffer, pH 3.6 (weight of sodium acetate trihydrate is 0.27216 g in 100 mL ACS water, adjusted to the desired pH using HCl). These three solutions (TPTZ, FeCl3, acetate buffer) are mixed in a 1:1:10 ratio. A 245 μL volume of the mixed solution is pipetted into a plastic cuvette with subsequent addition of a 5 μL sample (gallic acid, Trolox®). Absorbance is measured at primary λ 593 nm wavelength. Different concentrations of Trolox® was used to make a standard curve and samples are compared to standard curve. Total antioxidants are calculated using the following equation

$$Antioxidants ( n mol / mg ) = \frac{n moles present in the sample \times total sample extraction volume}{total wt of the sample \times volume used for measurement}$$

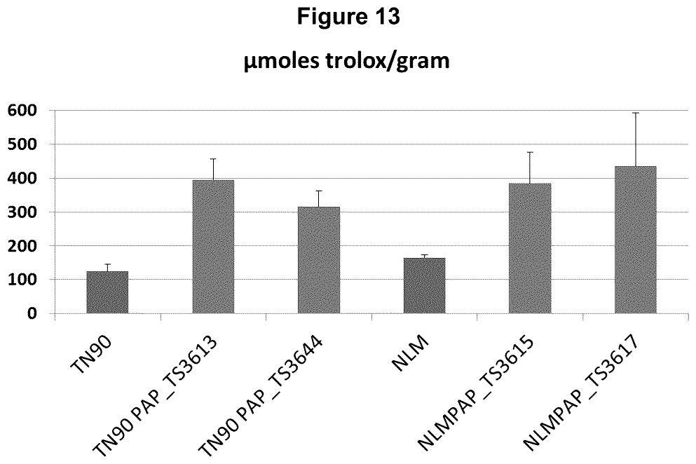

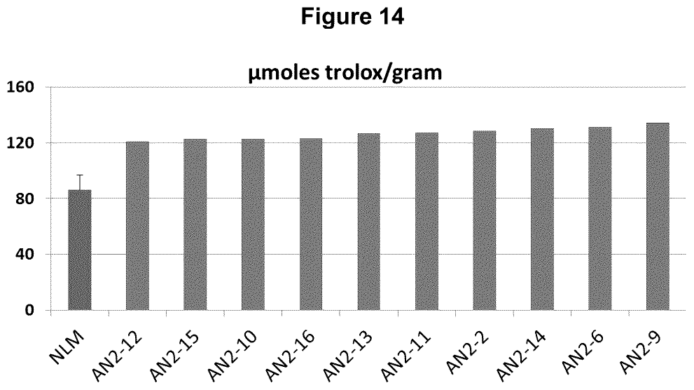

Modified tobacco plants overexpressing AtPAP1 show a significantly increased antioxidant capacity as measured by FRAP analysis compared to the unmodified controls (P<0.01) (FIG. 13).

### Example 8. Secondary Metabolite Accumulation in Field Grown AtPAP1 Overexpressing Plants

A secondary metabolite accumulation analysis is conducted on greenhouse grown AtPAP1 overexpressing plants. AtPAP1 overexpression constructs are transformed into Narrow Leaf Madole (NLM) tobacco plants as described in Example 1. Modified and unmodified control plants are grown in a greenhouse under standard conditions. Plants are topped at flowering and leaves for analysis are collected two weeks later from two independently modified NLM plants (D1 and D2) and one unmodified NLM plant. A set of Benzenoids, Flavonoids, and Phenylpropanoids show significantly increased levels in modified plants compared to unmodified plants (P<0.01) (See Table 5).

TABLE 5
Secondary metabolite accumulation in 35:AtPAP1 overexpressing
Narrow Leaf Madole tobacco plants.

| Pathway | Biochemical Name | D1/DC | D2/DC | D1 | D2 | DC |
|---|---|---|---|---|---|---|
| Benzenoids | protocatechuic acid-3-glucoside | 109.21* | 101.57* | 5.56 | 5.17 | 0.05 |
| | 4-hydroxybenzoate | 12.26* | 11.42* | 1.37 | 1.27 | 0.11 |
| | gentisic acid-5-glucoside | 5.98* | 6.23* | 2.16 | 2.25 | 0.36 |
| | salicylate-glucoside | 5.09** | 5.85* | 0.95 | 1.10 | 0.19 |
| | benzoyl-O-glucose | 4.62* | 4.6* | 0.27 | 0.27 | 0.06 |
| | salicylate | 2.22* | 2.81* | 0.63 | 0.80 | 0.28 |
| | benzoate | 1.49 | 2.07* | 0.52 | 0.72 | 0.35 |
| Flavonoids | rutinose | 99.63* | 93.99* | 41.90 | 39.52 | 0.42 |
| | naringenin | 5.26* | 3.79* | 1.19 | 0.86 | 0.23 |
| | quercetin 3-galactoside | 2.54 | 3.11 | 0.14 | 0.17 | 0.05 |
| Phenylpropanoids | coumaroylquinate (4) | 92.06* | 45.21* | 21.81 | 10.71 | 0.24 |
| | coumaroylquinate (2) | 16.17* | 12.87* | 10.70 | 8.52 | 0.66 |
| | chlorogenate | 9.15* | 6.59* | 7.55 | 5.44 | 0.83 |
| | vanillate | 5.95* | 7.25* | 0.39 | 0.47 | 0.06 |
| | cryptochlorogenic acid | 4.74* | 3.93* | 2.18 | 1.80 | 0.46 |
| | coumaroylquinate (5) | 5.74* | 5.86* | 5.44 | 5.55 | 0.95 |
| | coumaroylquinate (3) | 3.2* | 2.99* | 2.32 | 2.17 | 0.72 |
| | dihydroferulic acid | 2.45** | 2.57* | 0.74 | 0.78 | 0.30 |
| | vanillin | 2.37* | 1.82** | 0.77 | 0.60 | 0.33 |

D1 and D2 represent individual plants from independently transformed lines and DC represents an unmodified control plant.
Highly significant differences as compared to the unmodified control plant (P < 0.01) are indicated with*.
Significant differences as compared to the unmodified control plant (P < 0.05) are indicated with**.

### Example 9. Expression of Additional Genes to Modulate TSNA Levels

Transformation vectors and modified tobacco plants are generated to overexpress full-length coding sequences from tobacco genes (e.g., SEQ ID NOs: 24-42, 44, 45, and 53 to 58) or non-tobacco origin genes (e.g., SEQ ID NOs: 43 and 46) that promote or are involved in the production or accumulation of one or more antioxidants (see Table 6). The overexpression of transcription factors that promote or are involved in the production or accumulation of one or more antioxidants is described below. Nucleotide (Table 7) and protein (Table 8) identity scores are shown comparing the transcription factors used in this study. Exemplary genes and their sequences are listed in the sequence listing and Table 9.

NtAN2, SEQ ID NO: 30, is incorporated into a p45-2-7 transformation vector, and modified tobacco plants are generated, according to Example 1. Modified NLM tobacco plants (T0 and T1 generation) and control tobacco plants are grown for 4-6 weeks after transplantation to soil, harvested, and cured in PGC chambers. Cured leaf samples are prepared for evaluation of TSNAs, oxidative degradation potential, alkaloids, and nitrites/nitrates as described in Examples 2 to 5. A FRAP assay is used to determine antioxidant capacity in T0 plants as described in Example 7. Increased antioxidant capacity is detected in individual field grown T0 plants compared to the average antioxidant capacity determined for at least five unmodified Narrow leaf Madole plants (FIG. 14).

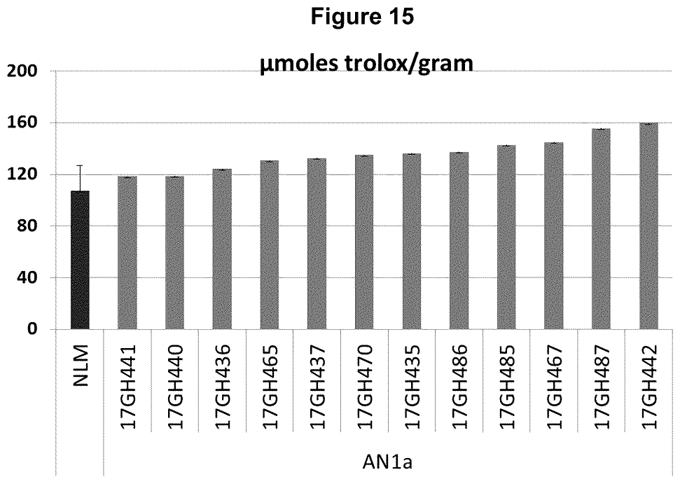

NtAN1a, SEQ ID NO: 28, is incorporated into a p45-2-7 transformation vector, and modified tobacco plants are generated, according to Example 1. Modified NLM tobacco plants (T0 and T1 generation) and control tobacco plants are grown for 4-6 weeks after transplantation to soil, harvested, and cured in PGC chambers. Cured leaf samples are prepared for evaluation of TSNAs, oxidative degradation potential, alkaloids, and nitrites/nitrates as described in Examples 2 to 5. A FRAP assay is used to determine antioxidant capacity in T0 plants as described in Example 7. Increased antioxidant capacity is detected in individual greenhouse grown T0 plants compared to the average antioxidant capacity determined for at least five unmodified Narrow leaf Madole plants (FIG. 15).

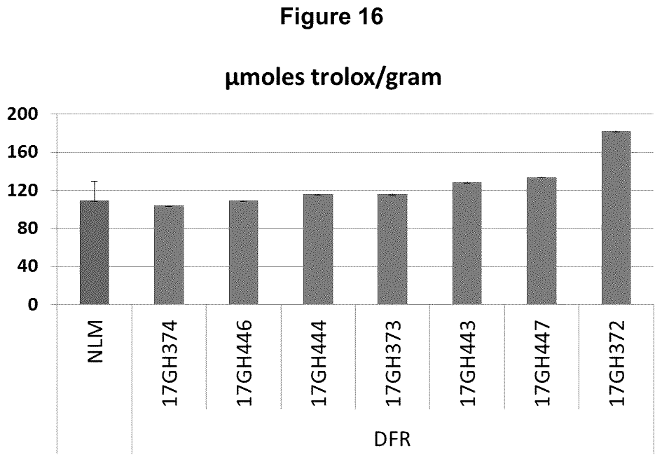

NtDFR, SEQ ID NO: 37, is incorporated into a p45-2-7 transformation vector, and modified tobacco plants are generated, according to Example 1. Modified NLM tobacco plants (T0 and T1 generation) and control tobacco plants are grown for 4-6 weeks after transplantation to soil, harvested, and cured in PGC chambers. Cured leaf samples are prepared for evaluation of TSNAs, oxidative degradation potential, alkaloids, and nitrites/nitrates as described in Examples 2 to 5. A FRAP assay is used to determine antioxidant capacity in T0 plants as described in Example 7. Increased antioxidant capacity is detected in individual greenhouse grown T0 plants compared to the average antioxidant capacity determined for at least five unmodified Narrow leaf Madole plants (FIG. 16).

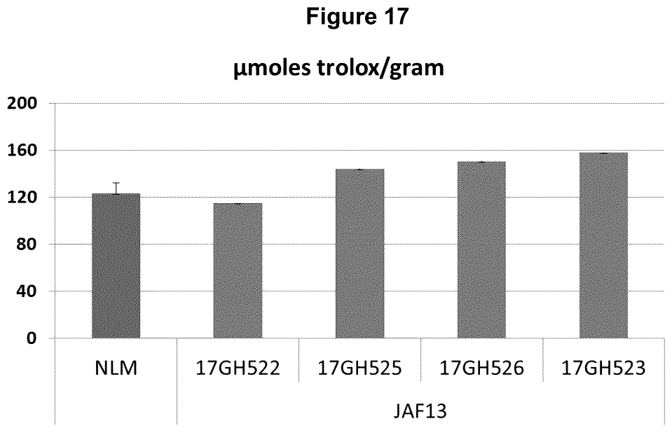

NtJAF13, SEQ ID NO: 33, is incorporated into a p45-2-7 transformation vector, and modified tobacco plants are generated, according to Example 1. Modified NLM tobacco plants (T0 and T1 generation) and control tobacco plants are grown for 4-6 weeks after transplantation to soil, harvested, and cured in PGC chambers. Cured leaf samples are prepared for evaluation of TSNAs, oxidative degradation potential, alkaloids, and nitrites/nitrates as described in Examples 2 to 5. A FRAP assay is used to determine antioxidant capacity in T0 plants as described in Example 7. Increased antioxidant capacity is detected in individual greenhouse grown T0 plants compared to the average antioxidant capacity determined for at least five unmodified Narrow leaf Madole plants (FIG. 17).

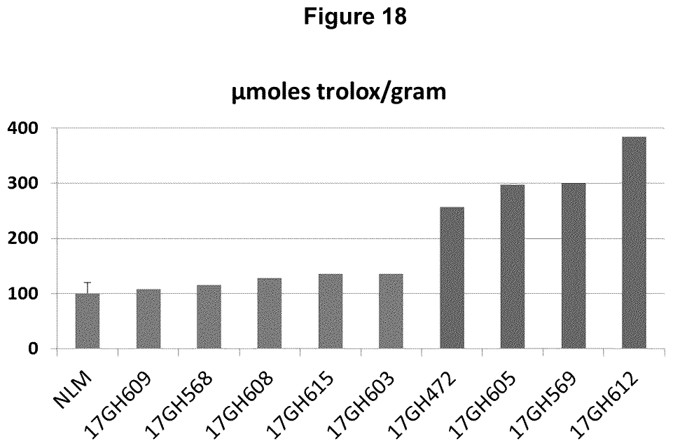

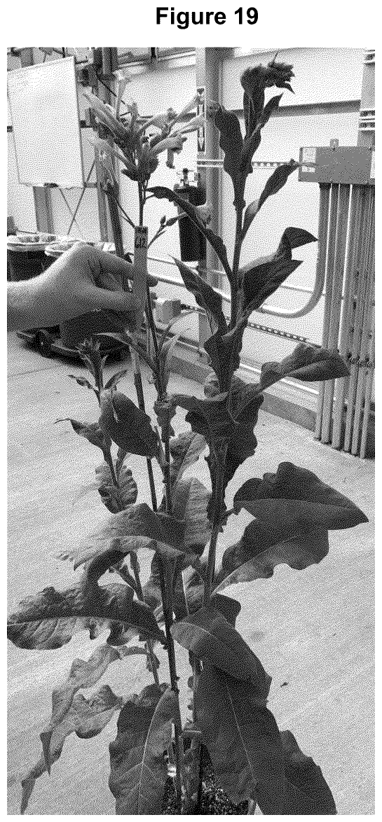

NtMYB3, SEQ ID NO: 36, is incorporated into a p45-2-7 transformation vector, and modified tobacco plants are generated, according to Example 1. Modified tobacco plants (T0 and T1 generation) and control tobacco plants are grown for 4-6 weeks after transplantation to soil, harvested, and cured in PGC chambers. Cured leaf samples are prepared for evaluation of TSNAs, oxidative degradation potential, alkaloids, and nitrites/nitrates as described in Examples 2 to 5. A FRAP assay is used to determine antioxidant capacity in T0 plants as described in Example 7. Increased antioxidant capacity is detected in individual greenhouse grown T0 plants compared to the average antioxidant capacity determined for at least five unmodified Narrow leaf Madole plants (FIG. 18). Plants overexpressing NtMYB3 show a normal leaf color in the TO generation (FIG. 19).

TABLE 6
A list of plant-origin antioxidants that can be used to reduce TSNAs.

| Chemical Classes | Compounds | Source of the Species |
|---|---|---|
| Anthocyanidin | Delphnidin | Tobacco, Arabidopsis. Cabbage, potato or petunia |
| | Cyanidin | Tobacco, Arabidopsis. Cabbage, potato or petunia |
| | Procyanidin | Tobacco, Arabidopsis. Cabbage, potato or petunia |
| | Prodelphinidin | Tobacco, Arabidopsis. Cabbage, potato or petunia |
| Flavanone | Hesperetin | Citrus or related species |
| | Naringenin | Citrus or related species |
| Flavanol | Catechin | Tobacco or other related species |
| | Epicatechin | Tobacco or other related species |
| Flavone | Apigenin | Parsley, tobacco or other related species |
| | Luteonin | Parsley, tobacco or other related species |
| Flavonol | Quercetin | Red kidney bean or other related species |
| | Myricetin | Red kidney bean or other related species |
| | Rutin | Tobacco, Red kidney bean or other related species |
| Isoflavone | Genistein | Soybean or other related species |
| | Daidzein | Soybean or other related species |
| HydroxybenzoicAcid | Gallic acid | Tobacco, oak or other related species |
| | Vanillic acid | Tobacco, Acai or other related species |
| | Protocatechuic | Tobacco, Hibiscus or other related species |
| | acid | |
| Hydroxycinnamic acid | Ferunic acid | Tobacco or other related species |
| | Cinnamic acid | Tobacco or other related species |
| | Coumeric acid | Tobacco or other related species |
| | Chlorogenic acid | Tobacco or other related species |
| | Coffeic acid | Tobacco or other related species |
| | Ferulic acid | Tobacco or other related species |
| Ellagitannin | Sanguiin | Raspberry or other related species |
| Stibene | Resveratrol | Grape or other related species |
| Lignan | Sesamin | Sesame or other related species |
| carotenoids | Caretonoids | Tobacco or carrots |
| | Vitamin C | Tobacco or carrots |
| Glycyrrhzin | | Licorice |

TABLE 7
Nucleotide sequence comparison between selected
transcription factors. Percent identity is shown.
Nucleotide identity

| | AtPAP1 | NtAN1 | NtAN2 | NtMYB3 | NtJAF13 |
|---|---|---|---|---|---|
| AtPAP1 | | 15.12 | 49.35 | 46.99 | 17.72 |
| NtAN1 | 15.12 | | 13.29 | 17.4 | 46.75 |
| NtAN2 | 49.35 | 13.29 | | 39.98 | 14.24 |
| NtMYB3 | 46.99 | 17.4 | 39.98 | | 19.4 |
| NtJAF13 | 17.72 | 46.75 | 14.24 | 19.4 | |

TABLE 8
Amino acid sequence comparison between selected
transcription factors. Percent identity is shown.
Amino Acid identity

| | AtPAP1 | NtAN1 | NtAN2 | NtMYB3 | NtJAF13 |
|---|---|---|---|---|---|
| AtPAP1 | | 6.8 | 46.3 | 33.67 | 7.5 |
| NtAN1 | 6.8 | | 5.77 | 7.4 | 30.64 |
| NtAN2 | 46.3 | 5.77 | | 32.78 | 7.19 |
| NtMYB3 | 33.67 | 7.4 | 32.78 | | 9.25 |
| NtJAF13 | 7.5 | 30.64 | 7.19 | 9.25 | |

TABLE 9
Nucleotide and protein sequences.

| | | | Protein | Coding |
|---|---|---|---|---|
| Target | | | SEQ ID | SEQ ID |
| Antioxidant | Gene Function annotation | Source | No. | No. |
| Anthocyanin | Putative alcohol dehydrogenase; [Solanum | tobacco | 1 | 24 |
| | lycopersicum (Tomato) (Lycopersicon esculentum).] | | | |
| Anthocyanin | 1-O-acylglucose:anthocyanin-O-acyltransferase; | tobacco | 2 | 25 |
| | [Clitoria ternatea (Butterfly pea).] | | | |
| Chlorogenic | 4-coumarate:CoA ligase; [Ipomoea batatas (Sweet | tobacco | 3 | 26 |
| acid | potato) (Convolvulus batatas).]. Also called 4CL | | | |
| Chlorogenic | 4-coumarate:CoA ligase-like; [Nicotiana sylvestris | tobacco | 4 | 27 |
| acid | (Wood tobacco) (South American tobacco).]. Also | | | |
| | called 4CL. | | | |
| Anthocyanin | Anthocyanin 1a; [Nicotiana tabacum (Common | tobacco | 5 | 28 |
| | tobacco).]. Also called AN1a. | | | |
| Anthocyanin | Anthocyanin 1b; [Nicotiana tabacum (Common | tobacco | 6 | 29 |
| | tobacco).]. Also called AN1b. | | | |
| Anthocyanin | Anthocyanin 2; [Nicotiana tomentosiformis | tobacco | 7 | 30 |
| | (Tobacco).] | | | |
| Anthocyanin | anthocyanidin synthase 2 [Nicotiana tabacum]. Also | tobacco | 8 | 31 |
| | called ANS2. | | | |
| Anthocyanin | leucoanthocyanidin dioxygenase [Nicotiana | tobacco | 9 | 32 |
| | tabacum] | | | |
| Anthocyanin | BHLH transcription factor JAF13; [Petunia hybrida | tobacco | 10 | 33 |
| | (Petunia).] | | | |
| Ferulic acid | Nicotiana tabacum caffeic acid O-methyltransferase | tobacco | 11 | 34 |
| | II gene | | | |
| chlorogenic | trans-cinnamate 4-monooxygenase-like [Nicotiana | tobacco | 12 | 35 |
| acid | tomentosiformis], Also called C4H. | | | |
| Anthocyanin | transcription factor MYB3-like [Nicotiana tabacum]; | tobacco | 13 | 36 |
| | tobacco homolog of AtPAP1 | | | |
| Anthocyanin | Nicotiana tabacum dihydroflavonol-4-reductase | tobacco | 14 | 37 |
| | (LOC107797232) | | | |
| Anthocyanin | Nicotiana tabacum NtDFR2 gene for | tobacco | 15 | 38 |
| | dihydroflavonol-4-reductase | | | |
| Anthocyanin | Nicotiana tabacum myb-related protein 308-like | tobacco | 16 | 39 |
| | (LOC107782378), mRNA-XM_016603259. | | | |
| Chlorogenic | Nicotiana tabacum shikimate O- | tobacco | 17 | 40 |
| acid | hydroxycinnamoyltransferase-like; also called HCT. | | | |
| Chlorogenic | Nicotiana tabacum mRNA for hydroxycinnamoyl | tobacco | 18 | 41 |
| acid | CoA quinate transferase (hqt gene); also called HQT. | | | |
| Anthocyanin, | Nicotiana tabacum phenylalanine ammonia lyase | tobacco | 19 | 42 |
| CGA, ferulic | (tpa1) gene; also called PAL. | | | |
| acid, | | | | |
| cinnamate, | | | | |
| coumarate | | | | |
| caffeic acid | | | | |
| Anthocyanin | Arabidopsis thaliana ttg1 gene; WD40. | Arabidopsis | 20 | 43 |
| carotenoids | phytoene synthase 1 [Nicotiana tabacum] | tobacco | 21 | 44 |
| carotenoids | phytoene synthase 2, chloroplastic [Nicotiana | tobacco | 22 | 45 |
| | sylvestris] | | | |
| Anthocyanin | Production of anthocyanin pigment 1; PAP1. | Arabidopsis | 23 | 46 |
| Flavonoids | Phenylalanine ammonia-lyase 4 (NtPAL4) | tobacco | 47 | 53 |
| and | | | | |
| anthocyanins | | | | |
| Flavonoids | Phenylalanine ammonia-lyase 2 (NtPAL2) | tobacco | 48 | 54 |
| and | | | | |
| anthocyanins | | | | |
| Flavonoids | Chalcone synthase (NtCHS) | tobacco | 49 | 55 |
| and | | | | |
| anthocyanins | | | | |
| Flavonoids | Flavonol 3-hydratase (NtF3H) | tobacco | 50 | 56 |
| and | | | | |
| anthocyanins | | | | |
| | Arogenate dehydrogenase 1 (NtADT1) | tobacco | 51 | 57 |
| Chlorogenic | Arogenate dehydrogenase 2 (NtADT2) | tobacco | 52 | 58 |
| acid, | | | | |
| Flavonoids | | | | |
| and | | | | |
| anthocyanins | | | | |

### Example 10: Creation of Cisgenic Constructs to Modulate TSNA Levels

Cisgenic constructs are created to constitutively express AtPAP1, NtAN2, and NtAN1a. Tobacco native Ubiquitin (Ubi-4) or Tubulin (Tub) promoters are used in conjunction with a tobacco native heat shock protein (HSP) terminator. Sequences are incorporated into a p45-2-7 transformation vector, and modified tobacco plants are generated, according to Example 1. Constructs encoding Ubi4-P:PAP1-HSP-T (SEQ ID NO:59), Ubi4-P:NtAN2-HSP-T (SEQ ID NO: 60), Tub-P:NtAN2-HSP-T (SEQ ID NO:61), Ubi4-P:NtAN2-HSP-T:Tub-P:NtAN2-HSP-T (SEQ ID NO:62), and Ubi4-P:NtAN1a-HSP-T:Tub-P:NtAN2-HSP-T (SEQ ID NO:63) are transformed into tobacco plants. The presence of the cisgenic construct in a transformed plant is confirmed using amplicon sequencing. Modified tobacco plants (T0 and T1 generation) and control tobacco plants are grown for 4-6 weeks after transplantation to soil, harvested, and cured in PGC chambers. Cured leaf samples are prepared for evaluation of TSNAs, oxidative degradation potential, alkaloids, and nitrites/nitrates as described in Examples 2 to 7.

### Example 11: A Combination Approach for Further Reduction of TSNAs

Three nicotine demethylase genes, known as CYP82E4, CYP82E5, and CYP82E10, mediate nornicotine biosynthesis in Nicotiana tabacum. Triple knockout mutants (cyp82e4, cyp82e5, cyp82e10) exhibit a dramatic reduction of nornicotine and consequently a reduction of NNN. A combination strategy is taken to combine nicotine demethylase mutants and the approach provided in Examples 2 to 7 to achieve a further TSNA reduction. A cyp82e4, cyp82e5, cyp82e10 triple mutant is transformed with one or more constructs described in Example 9 to increase antioxidant levels. Alternatively, cyp82e4, cyp82e5, cyp82e10 triple mutants are crossed with mutant or transgenic tobacco having elevated antioxidant levels described in Example 2.

1-45. (canceled)

46. A modified tobacco plant comprising:
a reduced level of one or more tobacco-specific nitrosamines (TSNAs);
an increased level of activity in a polypeptide comprising a sequence at least 90% identical to SEQ ID NO: 13 or an increased amount of the polypeptide; and
an increased level of one or more antioxidants;
wherein the reduced and increased levels are compared to an unmodified control tobacco plant of the same variety when grown under comparable conditions, and wherein the increased level of activity or increased amount of the polypeptide is a result of a genetic modification in the modified tobacco plant.

47. The modified tobacco plant of claim 46, wherein a cured leaf of the modified tobacco plant comprises an increased level of oxygen radical absorbance capacity (ORAC) compared to a cured leaf of the unmodified control tobacco plant.

48. The modified tobacco plant of claim 46, wherein a cured leaf of the modified tobacco plant comprises less than 2 parts per million (ppm) total TSNAs.

49. The modified tobacco plant of claim 46, wherein a cured leaf of the modified tobacco plant comprises less than 0.08 ppm 4-methylnitrosoamino-1-(3-pyridyl)-1-butanone (NNK), wherein a level of total TSNAs is measured based on a freeze-dried cured leaf sample using liquid chromatograph with tandem mass spectrometry (LC/MS/MS).

50. The modified tobacco plant of claim 46, wherein the one or more antioxidants are selected from the group consisting of anthocyanidin, flavanone, flavanol, flavone, flavonol, isoflavone, hydroxybenzoic acid, hydroxycinnamic acid, ellagitannin, stibene, lignan, carotenoids, and glycyrrhzin.

51. The modified tobacco plant of claim 46, wherein the one or more TSNAs are selected from the group consisting of N′-nitrosonornicotine (NNN), NNK, N′-nitrosoanatabine (NAT), and any combination thereof.

52. The modified tobacco plant of claim 46, wherein the modified tobacco plant is selected from the group consisting of a flue-cured variety, a Burley variety, a Maryland variety, a dark variety, and an Oriental variety.

53. The modified tobacco plant of claim 46, wherein the modified tobacco plant comprises a transgene or a mutation in a tobacco genome.

54. The modified tobacco plant of claim 53, wherein the level of the one or more TSNAs is reduced by at least 50% compared to a control tobacco plant not comprising the transgene or the mutation.

55. The modified tobacco plant of claim 53, wherein the transgene encodes a polypeptide comprising a sequence having at least 95% identity to SEQ ID NO. 13.

56. A modified tobacco plant comprising:
a reduced level of nitrite;
an increased level of activity in a polypeptide comprising a sequence at least 90% identical to SEQ ID NO: 13 or an increased amount of the polypeptide; and
an increased level of one or more antioxidants;
wherein the reduced and increased levels are compared to an unmodified control tobacco plant of the same variety when grown under comparable conditions, and wherein the increased level of activity or increased amount of the polypeptide is a result of a genetic modification in the modified tobacco plant.

57. The modified tobacco plant of claim 56, wherein a cured leaf of the modified tobacco plant comprises an increased level of ORAC compared to a cured leaf of the unmodified control tobacco plant.

58. The modified tobacco plant of claim 56, wherein a cured leaf of the modified tobacco plant comprises less than 2 ppm total TSNAs.

59. The modified tobacco plant of claim 56, wherein a cured leaf of the modified tobacco plant comprises less than 0.08 ppm NNK, wherein a level of total TSNAs is measured based on a freeze-dried cured leaf sample using LC/MS/MS.

60. The modified tobacco plant of claim 56, wherein the one or more antioxidants are selected from the group consisting of anthocyanidin, flavanone, flavanol, flavone, flavonol, isoflavone, hydroxybenzoic acid, hydroxycinnamic acid, ellagitannin, stibene, lignan, carotenoids, and glycyrrhzin.

61. The modified tobacco plant of claim 56, wherein the one or more TSNAs are selected from the group consisting of NNN, NNK, NAT, and any combination thereof.

62. The modified tobacco plant of claim 56, wherein the modified tobacco plant is selected from the group consisting of a flue-cured variety, a Burley variety, a Maryland variety, a dark variety, and an Oriental variety.

63. The modified tobacco plant of claim 56, wherein the modified tobacco plant comprises a transgene or a mutation in a tobacco genome.

64. The modified tobacco plant of claim 63, wherein the level of the one or more TSNAs is reduced by at least 50% compared to a control tobacco plant not comprising the transgene or the mutation.

65. The modified tobacco plant of claim 63, wherein the transgene encodes a polypeptide comprising a sequence having at least 95% identity to SEQ ID NO. 13.
